(19) United States
(12) Patent Application Publication
(10) Pub. No.: US 2017/0232083 A1
(43) Pub. Date: Aug. 17, 2017

(54) METHODS OF DIAGNOSIS AND TREATMENT OF CELIAC DISEASE IN CHILDREN

(71) Applicant: ImmusanT, Inc., Cambridge, MA (US)

(72) Inventors: Robert P. Anderson, Shrewsbury, MA (US); Jason A. Tye-Din, Collingwood (AU)

(73) Assignee: ImmusanT, Inc., Cambridge, MA (US)

(21) Appl. No.: 15/306,154

(22) PCT Filed: Apr. 24, 2015
(86) PCT No.: PCT/US2015/027483

§ 371 Date: Oct. 24, 2016

Related U.S. Application Data
(60) Provisional application No. 62/115,925, filed on Feb. 13, 2015.
(60) Provisional application No. 62/115,897, filed on Feb. 13, 2015.
(60) Provisional application No. 62/116,052, filed on Feb. 13, 2015.
(60) Provisional application No. 62/116,002, filed on Feb. 13, 2015.
(60) Provisional application No. 62/115,963, filed on Feb. 13, 2015.
(60) Provisional application No. 62/116,027, filed on Feb. 13, 2015.
(60) Provisional application No. 62/082,832, filed on Nov. 21, 2014.
(60) Provisional application No. 62/057,152, filed on Sep. 29, 2014.
(60) Provisional application No. 62/057,163, filed on Sep. 29, 2014.
(60) Provisional application No. 62/043,386, filed on Aug. 28, 2014.
(60) Provisional application No. 62/043,390, filed on Aug. 28, 2014.
(60) Provisional application No. 62/043,395, filed on Aug. 28, 2014.
(60) Provisional application No. 62/014,666, filed on Jun. 19, 2014.
(60) Provisional application No. 62/014,676, filed on Jun. 19, 2014.
(60) Provisional application No. 62/014,681, filed on Jun. 19, 2014.
(60) Provisional application No. 62/014,373, filed on Jun. 19, 2014.
(60) Provisional application No. 62/014,401, filed on Jun. 19, 2014.
(60) Provisional application No. 62/011,794, filed on Jun. 13, 2014.
(60) Provisional application No. 62/011,493, filed on Jun. 12, 2014.
(60) Provisional application No. 62/011,540, filed on Jun. 12, 2014.
(60) Provisional application No. 62/011,508, filed on Jun. 12, 2014.
(60) Provisional application No. 62/011,561, filed on Jun. 12, 2014.
(60) Provisional application No. 62/011,566, filed on Jun. 12, 2014.
(60) Provisional application No. 62/009,090, filed on Jun. 6, 2014.
(60) Provisional application No. 62/009,146, filed on Jun. 6, 2014.
(60) Provisional application No. 61/984,043, filed on Apr. 25, 2014.
(60) Provisional application No. 61/983,989, filed on Apr. 24, 2014.
(60) Provisional application No. 61/983,993, filed on Apr. 24, 2014.
(60) Provisional application No. 61/983,981, filed on Apr. 24, 2014.
(60) Provisional application No. 61/984,028, filed on Apr. 24, 2014.

(51) Int. Cl. A61K 39/00; G01N 33/68; G01N 33/50

(52) U.S. Cl. CPC A61K 39/0008; G01N 33/5091; G01N 33/6893; A61K 2039/55; A61K 2039/577; G01N 2333/57; G01N 2800/50; G01N 2800/06

(57) ABSTRACT

Provided herein are compositions and methods for treating and/or identifying children having or suspected of having Celiac disease.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 06, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND

Celiac disease, also known as coeliac disease or Celiac sprue (Coeliac sprue), affects approximately 1% of people in Europe and North America. In many of those affected, Celiac disease is unrecognised, but this clinical oversight is now being rectified with greater clinical awareness. A gluten free diet is the only currently approved treatment for Celiac disease, and because regular ingestion of as little as 50 mg of gluten (equivalent to 1/100th of a standard slice of bread) can damage the small intestine; chronic inflammation of the small bowel is commonplace in subjects on a gluten free diet. Persistent inflammation of the small intestine has been shown to increase the risk of cancer, osteoporosis and death. As gluten is so widely used, for example, in commercial soups, sauces, ice-creams, etc., maintaining a gluten-free diet is difficult. Proper diagnosis and treatment of children having Celiac disease is important for improving the quality of life at an earlier age.

### SUMMARY

The disclosure relates to compositions and methods for identifying and/or treating children having or at risk of having Celiac disease. As described herein, it has been surprisingly found that T cell epitopes dominant in adults also cause a T cell response in children having Celiac disease. In some embodiments, these T cell epitopes comprise PFPQPELPY (SEQ ID NO: 1), PQPELPYPQ (SEQ ID NO: 2), PFPQPEQPF (SEQ ID NO: 3), PQPEQPFPW (SEQ ID NO: 4), and PIPEQPQPY (SEQ ID NO: 5). In some embodiments, these T cell epitopes comprise PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 5) and EQPIPEQPQ (SEQ ID NO: 6). It is, therefore, expected that diagnostic and treatment methods involving use of peptides comprising these dominant T cell epitopes, which were previously validated in adults, will also be useful in children.

Some aspects of the disclosure relate to a method for treating Celiac disease in a child, the method comprising administering to a child having Celiac disease an effective amount of a composition comprising one or more peptides comprising an adult immunodominant epitope. In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the first, second, and/or third peptide are each independently 8-50 amino acids in length. In some embodiments, the first peptide comprises LQPFPQPQLPYPQPQ (SEQ ID NO: 7); the second peptide comprises QPFPQPQQPFPWQP (SEQ ID NO: 8); and the third peptide comprises PQQPIPQQPQPYPQQ (SEQ ID NO: 9). In some embodiments, the first, second, and/or third peptide are each independently 15-30 amino acids in length. In some embodiments, the first peptide comprises the amino acid sequence ELQPFPQPELPYPQPQ (SEQ ID NO: 10), wherein the N-terminal glutamate is a pyroglutamate and the C-terminal glutamine is amidated; the second peptide comprises the amino acid sequence EQPFPQPEQPFPWQP (SEQ ID NO: 11), wherein the N-terminal glutamate is a pyroglutamate and the C-terminal proline is amidated; and the third peptide comprises the amino acid sequence EPEQPIPEQPQPYPQQ (SEQ ID NO: 12), wherein the N-terminal glutamate is a pyroglutamate and the C-terminal glutamine is amidated. In some embodiments, the amino acid sequence of the first peptide is ELQPFPQPELPYPQPQ (SEQ ID NO: 10), wherein the N-terminal glutamate is a pyroglutamate and the C-terminal glutamine is amidated; the amino acid sequence of the second peptide is EQPFPQPEQPFPWQP (SEQ ID NO: 11), wherein the N-terminal glutamate is a pyroglutamate and the C-terminal proline is amidated; and the amino acid sequence of the third peptide is EPEQPIPEQPQPYPQQ (SEQ ID NO: 12), wherein the N-terminal glutamate is a pyroglutamate and the C-terminal glutamine is amidated. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the composition comprises 50 micrograms of the first peptide and an equimolar amount of each of the second and third peptides. In some embodiments, the composition comprises 26.5 nmol of each of the first, second, and third peptides. In some embodiments, the composition comprises 25 micrograms of the first peptide and an equimolar amount of each of the second and third peptides. In some embodiments, the composition comprises 13.2 nmol of each of the first, second, and third peptides. In some embodiments, the composition is administered intradermally. In some embodiments, the composition is administered as a bolus by intradermal injection. In some embodiments, the composition is formulated as a sterile, injectable solution. In some embodiments, the child is HLA-DQ2.5 positive. In some embodiments, the child is on a gluten-free diet.

Other aspects of the disclosure relate to a method for identifying a child as having or at risk of having Celiac disease, the method comprising determining a T cell response to a composition comprising one or more peptides comprising an adult immunodominant epitope in a sample comprising a T cell from the child; and assessing whether or not the child has or is at risk of having Celiac disease. In some embodiments, if the T cell response to the composition is elevated compared to a control T cell response, the child is identified as having or is at risk of having Celiac disease. In some embodiments, if the T cell response to the composition is reduced compared to the control T cell response or the same as the control T cell response, the child is identified as not having or not being at risk of having Celiac disease. In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the step of determining comprises contacting the sample with the composition and measuring a T cell response to the composition. In some embodiments, measuring a T cell response to the composition comprises measuring a level of a cytokine in the sample. In some embodiments, the cytokine is interferon-gamma. In some embodiments, measuring comprises an enzyme-linked immunosorbent assay (ELISA) or an enzyme-linked immunosorbent spot (ELISpot) assay. In some embodiments, the first, second, and/or third peptide are each independently 8-50 amino acids in length. In some embodiments, the first peptide comprises LQPFPQPQLPYPQPQ (SEQ ID NO: 7); the second peptide comprises QPFPQPQQPFPWQP (SEQ ID NO: 8); and the third peptide comprises PQQPIPQQPQPYPQQ (SEQ ID NO: 9). In some embodiments, the first, second, and/or third peptide are each independently 15-30 amino acids in length. In some embodiments, the first peptide comprises the amino acid sequence ELQPFPQPELPYPQPQ (SEQ ID NO: 10), wherein the N-terminal glutamate is a pyroglutamate and the C-terminal glutamine is amidated; the second peptide comprises the amino acid sequence EQPFPQPEQPFPWQP (SEQ ID NO: 11), wherein the N-terminal glutamate is a pyroglutamate and the C-terminal proline is amidated; and the third peptide comprises the amino acid sequence EPEQPIPEQPQPYPQQ (SEQ ID NO: 12), wherein the N-terminal glutamate is a pyroglutamate and the C-terminal glutamine is amidated. In some embodiments, the amino acid sequence of the first peptide is ELQPFPQPELPYPQPQ (SEQ ID NO: 10), wherein the N-terminal glutamate is a pyroglutamate and the C-terminal glutamine is amidated; the amino acid sequence of the second peptide is EQPFPQPEQPFPWQP (SEQ ID NO: 11), wherein the N-terminal glutamate is a pyroglutamate and the C-terminal proline is amidated; and the amino acid sequence of the third peptide is EPEQPIPEQPQPYPQQ (SEQ ID NO: 12), wherein the N-terminal glutamate is a pyroglutamate and the C-terminal glutamine is amidated. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the sample comprises whole blood or peripheral blood mononuclear cells. In some embodiments, the method further comprises administering a composition comprising wheat, rye, or barley, or a peptide thereof, to the child prior to determining the T cell response. In some embodiments, the composition comprising wheat, rye, or barley, or a peptide thereof, is administered to the child more than once prior to determining the T cell response. In some embodiments, the composition comprising wheat, rye, or barley is administered to the child at least once a day for three days. In some embodiments, the sample comprising the T cell is obtained from the child after the administration of the composition comprising wheat, rye, or barley, or a peptide thereof. In some embodiments, the composition comprising wheat, rye, or barley is administered to the child via oral administration. In some embodiments, the T cell response to the composition is measured 6 days after the oral administration. In some embodiments, the composition comprising wheat, rye, or barley, or a peptide thereof, is a foodstuff. In some embodiments, the method further comprises treating the child if identified as having or at risk of having Celiac disease or providing information to the child or the child's caregiver about a treatment. In some embodiments, the method further comprises a step of recommending a gluten-free diet if the child is identified as having or at risk of having Celiac disease or providing information to the child or the child's caregiver about such a diet. In some embodiments, the child is HLA-DQ2.5 positive.

The details of one or more embodiments of the disclosure are set forth in the description below. Other features or advantages of the present disclosure will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a graph showing the results from an ELISpot assay in which peripheral blood mononuclear cells obtained from children were contacted with gliadin or with a composition comprising Peptide 1: ELQPFPQPELPYPQPQ (SEQ ID NO: 10), Peptide 2: EQPFPQPEQPFPWQP (SEQ ID NO: 11), and Peptide 3: EPEQPIPEQPQPYPQQ (SEQ ID NO: 12). For each of peptide 1, 2, and 3, the N-terminal glutamate was a pyroglutamate and the carboxyl group of the C-terminal proline or glutamine was amidated. SFU/10⁶=number of spot forming units per 10⁶ cells.

FIG. 2 is a diagram showing a dose escalation study in children as described in Example 3.

FIG. 3 is a diagram showing a dose escalation study in children as described in Example 4.

FIGS. 4A-C are graphs showing that oral wheat challenge induces gluten-specific T cell responses in paediatric CD patients. Paediatric CD patients undertook 3 day oral wheat challenge and ELISpot's testing wheat derived proteins and peptides were tested for recognition. FIG. 4A) Responses to deamidated gliadin, peptide W02 containing DQ2.5-glia-a1a/a2, and peptide W03 containing DQ2.5-glia-w1/w2 on Day 0 prior to challenge and Day 6 after wheat challenge. Significant gluten-specific responses were observed on Day 6 only (p<0.05, Kruskal Wallis), and no difference seen for tetanus toxoid. FIG. 4B) Peptides W02 and W03 were tested in native and deamidated forms. Deamidation enhanced the response to peptide in all age groups, some statistically higher (p<0.05, Kruskal-Wallis). FIG. 4C) Responses to exemplary peptides (pE)QQPQQSFPEQERPF (SEQ ID NO:114), (pE)XPQQQQXPEQPQQF (SEQ ID NO:117), (pE)QQSEESEQPFQPQP (SEQ ID NO:119), (pE)QPPFSEEQEQPLPQ (SEQ ID NO:121), (pE)QPPFSEQQESPSFSQ (SEQ ID NO:123), (pE)GIIPEQPAQLEGI (SEQ ID NO:125), (pE)QPFRPEQPYPQPQP (SEQ ID NO:127), QPQQPQQSFPQQQRPF (SEQ ID NO:129), QQXSQPQXPQQQQXPQQPQQF (SEQ ID NO:131), QPQPFPQQSEQSQQPFQPQPF (SEQ ID NO:133), QQPPFSQQQQQPLPQ (SEQ ID NO:135), QQQQPPFQQQQSPFSQQQQ (SEQ ID NO:137), VQGQGIIQPQQPAQL (SEQ ID NO:139), and PFRPQQPYPQPQPQ (SEQ ID NO:141). Line depicts response cut-off.

FIG. 5 is a graph showing that low or negative responses to positive control antigens predicts lack of response to gluten peptides in wheat challenged CD patients. Patients were divided into those that were considered responders or non-responders to gluten-derived antigen in the ELISpot after oral wheat challenge. Responses to positive control antigens were compared. Dotted line depicts response cut-off. Median response is shown. GC responder is the left-most cluster of data for each of PHA, CEF, and TT. GC non-responder is the right-most cluster of data for each of PHA, CEF, and TT.

FIGS. 6A-E are graphs showing the effect of age, HLA-DQ2.5 zygosity, and time since diagnosis on gluten peptide T cell responses. Peptide W02 containing DQ2.5-glia-a1a/a2 and peptide W03 containing DQ2.5-glia-w1/w2 were tested in titrating doses in paediatric and adult CD patients following oral wheat challenge. Using the dose curves, EC50's were calculated and compared: FIG. 6A) Between age groups, FIG. 6B) Between homozygous and heterozygous individuals, and FIG. 6E) Between patients diagnosed less than 2 years prior to gluten challenge or over two years. FIG. 6C) ELISpot response magnitude in homozygous and heterozygous individuals. FIG. 6D) ELISpot response magnitude divided into age groups. Median and interquartile ranges are shown (*p<0.05, Kruskal-Wallis).

FIG. 7 is a diagram showing T cell clone promiscuity. Positive responses are shaded for wheat are shown above the top dotted line, positive responses for barley are shown between the top and bottom dotted line, and positive responses for rye are shown below the bottom dotted line. Non-reactive peptides were removed.

FIGS. 8A-8C show that oral wheat challenge induces gluten-specific T cell responses in children with CD. Pediatric CD volunteers undertook 3 day oral wheat challenge and T cell responses to wheat-derived proteins and peptides were assessed by IFN-γ ELISpot. FIG. 8A is a graph that shows responses to deamidated gliadin, peptide W02 containing DQ2.5-glia-a1a/a2, and peptide W03 containing DQ2.5-glia-w1/w2 on Day 0 prior to and Day 6 after wheat challenge (background subtracted). Significant gluten-specific responses were observed on Day 6 only (p<0.05, Kruskal Wallis), and no difference seen for tetanus toxoid. FIG. 8B is two graphs that show peptides W02 and W03 tested in native and deamidated forms. Deamidation enhanced the response to peptide in all ages (3-5 n=4; 6-10 n=7; 11-18 n=8), some statistically significant (p<0.05, Kruskal-Wallis). FIG. 8C is a graph that shows polyclonal responses to peptides immunogenic to TCLs ((pE)QQPQQSFPEQERPF (SEQ ID NO:114), (pE)XPQQQQXPEQPQQF (SEQ ID NO:117), (pE)QQSEESEQPFQPQP (SEQ ID NO:119), (pE)QPPFSEEQEQPLPQ (SEQ ID NO:121), (pE)QPPFSEQQESPSFSQ (SEQ ID NO:123), (pE)GIIPEQPAQLEGI (SEQ ID NO:125), (pE)QPFRPEQPYPQPQP (SEQ ID NO:127), QPQQPQQSFPQQQRPF (SEQ ID NO:129), QQXSQPQXPQQQQXPQQPQQF (SEQ ID NO:131), QPQPFPQQSEQSQQPFQPQPF (SEQ ID NO:133), QQPPFSQQQQQPLPQ (SEQ ID NO:135), QQQQPPFQQQQSPFSQQQQ (SEQ ID NO:137), VQGQGIIQPQQPAQL (SEQ ID NO:139), and PFRPQQPYPQPQPQ (SEQ ID NO:141)) described by Vader et al. Dotted line depicts response cut-off.

FIG. 9 is a graph that shows that low or negative responses to positive control antigens predicted lack of response to gluten peptides in CD volunteers after wheat challenge. Volunteers were separated into responders (n=31) or non-responders (n=9) based on the IFN-γ ELISpot response to gluten after oral wheat challenge. Responses to positive control antigens PHA, CEF, and TT were compared. Dotted line depicts response cut-off. Median response with interquartile range is shown.

FIGS. 10A-10E are a series of graphs that show the effect of age, HLA-DQ2.5 zygosity, and time since diagnosis on gluten peptide T cell responses. Peptides W02 and W03 were assessed in a dose ranging study in pediatric and adult CD volunteers following oral wheat challenge. EC50's were calculated and compared: (FIG. 10A) Between age groups, (FIG. 10B) Between HLA-DQ2.5 homozygous (n=8) and heterozygous individuals (n=10-13), and (FIG. 10E) Between volunteers diagnosed less than 2 years prior to gluten challenge (n=8) or over two years (n=6-8). FIG. 10C shows ELISpot response magnitude in homozygous (n=7) and heterozygous (n=22) individuals. FIG. 10D shows ELISpot response magnitude divided by age (3-5 n=7); 6-10 n=10; 11-18 n=10). Median and interquartile ranges are shown (*p<0.05, Kruskal-Wallis or Mann-Whitney).

FIG. 11 shows a table of T cell clone promiscuity. T cell clones specific to DQ2.5-glia-α1a/α2 or DQ2.5-glia-ω1/ω2 were tested against wheat, barley, and, rye peptide libraries by IFN-γ ELISpots. Positive responses are shaded for wheat are shown above the top dotted line, positive responses for barley are shown between the top and bottom dotted line, and positive responses for rye are shown below the bottom dotted line.

### DETAILED DESCRIPTION OF THE INVENTION

Celiac disease occurs in genetically susceptible individuals who possess either HLA-DQ2.5 (encoded by the genes HLA-DQA1*05 and HLA-DQB1*02) accounting for about 90% of individuals, HLA-DQ2.2 (encoded by the genes HLA-DQA1*02 and HLA-DQB1*02), or HLA-DQ8 (encoded by the genes HLA-DQA1*03 and HLA-DQB1*0302). Without wishing to be bound by theory, it is believed that such individuals mount an inappropriate HLA-DQ2- and/or DQ8-restricted CD4+ T cell-mediated immune response to peptides derived from the aqueous-insoluble proteins of wheat flour, gluten, and related proteins in rye and barley. It was previously thought that the immune response of subjects with Celiac disease changed over time from childhood to adulthood, resulting in changes in T cell responses to different epitopes as subjects aged and had had Celiac disease for many years. Surprisingly, as described herein, it has been found that the T cell response is similar in children and adults, meaning that the same epitopes that activate T cells in adults also activate T cells in children.

Accordingly, the disclosure provides compositions and methods related to identifying and/or treating children having or at risk of having Celiac disease.

### Identification

In some aspects, the disclosure relates to methods for identifying (e.g., diagnosing) a child as having or at risk of having Celiac disease.

In some embodiments, the method comprises determining a T cell response to a peptide comprising an adult immunodominant epitope in a sample comprising a T cell from the child and identifying the child as (i) having or at risk of having Celiac disease if the T cell response to the peptide described herein is elevated compared to a control T cell response, or (ii) not having or not at risk of having Celiac disease if the T cell response to the peptide described herein is reduced compared to the control T cell response or the same as the control T cell response. In some embodiments, the peptide comprises a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the peptide comprises a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the peptide is in a composition and the composition comprises a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the peptide is in a composition and the composition comprises at least one peptide comprising at least one epitope as described herein, e.g., at least one of PFPQPELPY (SEQ ID NO: 1), PQPELPYPQ (SEQ ID NO: 2), PFPQPEQPF (SEQ ID NO: 3), PQPEQPFPW (SEQ ID NO: 4), (SEQ ID NO: 5) and EQPIPEQPQ (SEQ ID NO: 6).

In some embodiments, the method comprises determining a T cell response to a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6) or to a composition as described herein, e.g., comprising at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), in a sample comprising a T cell from the child; and identifying the child as (i) having or at risk of having Celiac disease if the T cell response to the peptide described herein is elevated compared to a control T cell response, or (ii) not having or at risk of having Celiac disease if the T cell response to the peptide described herein is reduced compared to the control T cell response or the same as the control T cell response.

T cells responses and methods of measuring T cell responses are described herein. In some embodiments, the step of determining comprises contacting the sample with a composition comprising a peptide comprising the adult immunodominant epitope and measuring a T cell response to the peptide described herein. In some embodiments, the peptide is as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the peptide is as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the peptide is in a composition and the composition comprises a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the peptide is in a composition and the composition comprises at least one peptide comprising at least one epitope as described herein, e.g., at least one of PFPQPELPY (SEQ ID NO: 1), PQPELPYPQ (SEQ ID NO: 2), PFPQPEQPF (SEQ ID NO: 3), PQPEQPFPW (SEQ ID NO: 4), (SEQ ID NO: 5) and EQPIPEQPQ (SEQ ID NO: 6). Without wishing to be bound by theory, it is believed that the peptide(s) described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), serves as an active component causing the activation and/or mobilization of CD4+ T cells in a child who has Celiac disease. Thus, in some embodiments, the T cell or T cell response referred to in any of the methods provided is a CD4+ T cell or CD4+ T cell response. In some embodiments, the child has or is at risk of having Celiac disease.

In some embodiments, a method described herein further comprises performing a challenge as described herein.

In some embodiments, a method described herein further comprises performing other testing, particularly if the child is identified as having or at risk of having Celiac disease. Other testing is described herein.

In some embodiments, a method described herein comprises a step of providing a treatment to a child identified as having or being at risk of having Celiac disease. In some embodiments, a method described herein comprises a step of providing information to the child or child's caregiver about a treatment. In some embodiments, a method described herein comprises a step of recommending a gluten free diet, or providing information about such a diet, if the child is identified as having or at risk of having Celiac disease. Information can be given orally or in written form, such as with written materials. Written materials may be in an electronic form. In some embodiments, treatment comprises administration of any of the compositions as described herein, such as a composition comprising at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, treatment comprises administration of a composition as described herein, such as a composition comprising at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6).

In some embodiments of any one of the methods provided, the method further comprises recording whether or not the child has celiac disease based on results of an assessing or measuring step. In some embodiments of any one of the methods provided herein, the method further comprises recording the level(s), the result(s) of the assessing and/or the treatment, or suggestion for treatment, based on the assessing.

### T Cell Responses and Measurement Thereof

Aspects of the disclosure relate to a determination or measurement of a T cell response in a sample comprising T cells from a child. In some embodiments, a composition comprising wheat, rye, and/or barley, or a peptide described herein (e.g., as a challenge described herein), is administered to a child and, preferably, is capable of activating a CD4⁺ T cell in a child, e.g., a child with Celiac disease. The term “activate” or “activating” or “activation” in relation to a CD4⁺ T cell refers to the presentation by an MHC molecule of an epitope on one cell to an appropriate T cell receptor on a second CD4⁺T cell, together with binding of a co-stimulatory molecule by the CD4⁺ T cell, thereby eliciting a “T cell response”, in this example a CD4⁺ T cell response. Such a T cell response can be measured ex vivo, e.g., by measuring a T cell response in a sample comprising T cells from the child.

As described herein, an elevated T cell response, such as an elevated CD4⁺ T cell response, from a sample comprising T cells from a child, e.g., after administration of a composition comprising wheat, rye, and/or barley or a peptide described herein, compared to a control T cell response can correlate with the presence or absence of Celiac disease in the child. Accordingly, aspects of the disclosure relate to methods that comprise determining or measuring a T cell response in a sample comprising T cells from a child, e.g., having or suspected of having Celiac disease.

In some embodiments, measuring a T cell response in a sample comprising T cells from a child comprises contacting the sample with a composition comprising a peptide comprising an adult immunodominant epitope. In some embodiments, the composition comprises at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). For example, whole blood or PBMCs obtained from a child who has been exposed to gluten (e.g., by a challenge as described herein or by administration of a peptide described herein) may be contacted with the composition comprising the peptide in order to stimulate T cells in the whole blood sample or PBMCs. In some embodiments, the composition comprises at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6).

Measuring a T cell response can be accomplished using any assay known in the art (see, e.g., Molecular Cloning: A Laboratory Manual, M. Green and J. Sambrook, Fourth Edition, Cold Spring Harbor Laboratory Press, Cold Spring Harbor, N.Y., 2012; Current Protocols in Molecular Biology, F. M. Ausubel, et al., Current Edition, John Wiley & Sons, Inc., New York). In some embodiments, measuring a T cell response comprises an MHC Class II tetramer assay, such as flow cytometry with MHC Class II tetramer staining (see, e.g., Raki M, Fallang L E, Brottveit M, Bergseng E, Quarsten H, Lundin K E, Sollid L M: Tetramer visualization of gut-homing gluten-specific T cells in the peripheral blood of Celiac disease patients. Proceedings of the National Academy of Sciences of the United States of America 2007; Anderson R P, van Heel D A, Tye-Din J A, Barnardo M, Salio M, Jewell D P, Hill A V: T cells in peripheral blood after gluten challenge in coeliac disease. Gut 2005, 54(9):1217-1223; Brottveit M, Raki M, Bergseng E, Fallang L E, Simonsen B, Lovik A, Larsen S, Loberg E M, Jahnsen F L, Sollid L M et al: Assessing possible Celiac disease by an HLA-DQ2-gliadin Tetramer Test. The American journal of gastroenterology 2011, 106(7):1318-1324; and Anderson R P, Degano P, Godkin A J, Jewell D P, Hill A V: In vivo antigen challenge in Celiac disease identifies a single transglutaminase-modified peptide as the dominant A-gliadin T cell epitope. Nature Medicine 2000, 6(3):337-342).

In some embodiments, measuring a T cell response in a sample comprising T cells from a child comprises measuring a level of at least one cytokine in the sample. In some embodiments, measuring a T cell response in a sample comprising T cells from a child comprises contacting the sample with a composition comprising a peptide, such as comprising at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), and measuring a level of at least one cytokine in the sample. In some embodiments, measuring a T cell response in a sample comprising T cells from a child comprises contacting the sample with a composition comprising at least one peptide comprising at least one epitope as described herein, e.g., at least one of PFPQPELPY (SEQ ID NO: 1), PQPELPYPQ (SEQ ID NO: 2), PFPQPEQPF (SEQ ID NO: 3), PQPEQPFPW (SEQ ID NO: 4), (SEQ ID NO: 5) and EQPIPEQPQ (SEQ ID NO: 6), and measuring a level of at least one cytokine in the sample. In some embodiments, the at least one cytokine is at least one pro-inflammatory cytokine such as IL-2, IFN-γ, IL-4, IL-5, IP-10, IL-13, and IL-17, e.g., by monocytes or granulocytes, as a result of secretion of these cytokines. In some embodiments, the at least one cytokine is IFN-γ or IP-10. In some embodiments, the at least one cytokine is IP-10. In some embodiments, the at least one cytokine is IFN-γ.

Interferon-y (IFN-γ, also called IFNG, IFG, and IFI) is a dimerized soluble cytokine of the type II class of interferons. IFN-γ typically binds to a heterodimeric receptor consisting of Interferon γ receptor 1 (IFNGR1) and Interferon γ receptor 2 (IFNGR2). IFN-γ can also bind to the glycosaminoglycan heparan sulfate (HS). IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops in a subject. In humans, the IFN-γ protein is encoded by the IFNG gene. The Genbank number for the human IFNG gene is 3458. Exemplary Genbank mRNA transcript IDs and protein IDs for IFN-γ are NM_000619.2 and NP_000610.2, respectively.

IFN-γ inducible protein-10 (IP-10, also referred to as C-X-C motif chemokine 10, CXCL10, small-inducible cytokine B10, SCYB10, C7, IFI10, crg-2, gIP-10, or mob-1) is a protein that in humans is encoded by the CXCL10 gene. IP-10 is a small cytokine belonging to the CXC chemokine family and binds to the chemokine receptor CXCR3. The Genbank ID number for the human CXCL10 gene is 3627. Exemplary Genbank mRNA transcript IDs and protein IDs for IP-10 are NM_001565.3 and NP_001556.2, respectively.

In some embodiments, measuring a T cell response comprises measuring a level of at least one cytokine. Levels of at least one cytokine include levels of cytokine RNA, e.g., mRNA, and/or levels of cytokine protein. In a preferred embodiment, levels of the at least one cytokine are protein levels.

Assays for detecting cytokine RNA include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine, e.g., IFN-γ, the sequence(s) being identifiable using the Genbank IDs described herein.

Assays for detecting protein levels include, but are not limited to, immunoassays (also referred to herein as immune-based or immuno-based assays, e.g., Western blot, ELISA, and ELISpot assays), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine, e.g., IFN-γ, the sequence(s) being identifiable using the Genbank IDs described herein. Other examples of protein detection and quantitation methods include multiplexed immunoassays as described for example in U.S. Pat. Nos. 6939720 and 8148171, and published U.S. Patent Application No. 2008/0255766, and protein microarrays as described for example in published U.S. Patent Application No. 2009/0088329.

Any suitable binding partner is contemplated herein. In some embodiments, the binding partner is any molecule that binds specifically to a cytokine as provided herein. A molecule is said to exhibit “specific binding” if it reacts or associates more frequently, more rapidly, with greater duration and/or with greater affinity with a particular target antigen than it does with alternative targets. As described herein, “binds specifically”, when referring to a protein, means that the molecule is more likely to bind to a portion of or the entirety of a protein to be measured than to a portion of or the entirety of another protein. In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically. Methods for producing peptide molecules and aptamers are well known in the art (see, e.g., published US Patent Application No. 2009/0075834, U.S. Pat. Nos. 7435542, 7807351, and 7239742). In some embodiments, the binding partner is any molecule that binds specifically to an IFN-γ mRNA. As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments, the binding partner that binds specifically to an mRNA is a nucleic acid, e.g., a probe. In a preferred embodiment, measuring a level of at least one cytokine comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939, 281, 6,410,252, and 7,575,870; Czerkinsky C, Nilsson L, Nygren H, Ouchterlony 0, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51 (12): 2415-8).

An exemplary ELISA involves at least one binding partner, e.g., an antibody or antigen-binding fragment thereof, with specificity for the at least one cytokine, e.g., IFN-γ. The sample with an unknown amount of the at least one cytokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the antigen is immobilized, the binding partner for the at least one cytokine is added, forming a complex with the immobilized at least one cytokine. The binding partner can be attached to a detectable label as described herein (e.g., a fluorophor or an enzyme), or can itself be detected by an agent that recognizes the at least one cytokine binding partner that is attached to a detectable label as described herein (e.g., a fluorophor or an enzyme). If the detectable label is an enzyme, a substrate for the enzyme is added, and the enzyme can elicit a chromogenic or fluorescent signal by acting on the substrate. The detectable label can then be detected using an appropriate machine, e.g., a fluorimeter or spectrophotometer, or by eye.

An exemplary ELISpot assay involves a binding agent for the at least one cytokine (e.g., an anti-IFN-γ) that is coated aseptically onto a PVDF (polyvinylidene fluoride)-backed microplate. Cells of interest (e.g., peripheral blood mononuclear cells) are plated out at varying densities, along with antigen (e.g., a peptide as described herein), and allowed to incubate for a period of time (e.g., about 24 hours). The at least one cytokine secreted by activated cells is captured locally by the binding partner for the at least one cytokine on the high surface area PVDF membrane. After the at least one cytokine is immobilized, a second binding partner for the at least one cytokine is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme can elicit a chromogenic or fluorescent signal by acting on the substrate. The detectable label can then be detected using an appropriate machine, e.g., a fluorimeter or spectrophotometer, or by eye.

In some embodiments, a level of at least one cytokine is measured using an ELISA. As an exemplary method, at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6) is dried onto the inner wall of a blood collection tube. A negative control tube containing no antigen is provided. A positive control tube containing a mitogen is also provided. Blood from a child is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of at least one cytokine (e.g., IFN-γ) present in the plasma. A standard ELISA assay as described above can then be used to detect the levels of the at least one cytokine present in each plasma sample. In some embodiments, a T cell response measurement in a sample obtained from the child after a challenge as described herein is detected using any of the methods above or any other appropriate method and is then compared to a control T cell response, e.g., a T cell response measurement in a sample obtained before challenge or a T cell response measurement in a sample from a control subject or subjects. Exemplary control T cell responses include, but are not limited to, a T cell response in a sample obtained from a diseased subject(s) (e.g., subject(s) with Celiac disease), a healthy subject(s) (e.g., subject(s) without Celiac disease) or a T cell response in a sample obtained from a child before or during a challenge as described herein. In some embodiments, a control T cell response is measured using any one of the methods above or any other appropriate methods. In some embodiments, the same method is used to measure T cell response in the sample of the child and the control sample.

In some embodiments, a T cell response is compared to a control T cell response. In some embodiments, if the control T cell response is a T cell response in a sample from a healthy control subject or subjects, then an elevated T cell response compared to the control T cell response is indicative that the child has or is at risk of having Celiac disease while a reduced or equal T cell response compared to the control T cell response is indicative that the child does not have or is not at risk of having Celiac disease. In some embodiments, if the control T cell response is a T cell response in a sample from the child before a challenge as described herein, then an elevated T cell response compared to the control T cell response is indicative that the child has or is at risk of having Celiac disease while a reduced or equal T cell response compared to the control T cell response is indicative that the child does not have or is not at risk of having Celiac disease.

An elevated T cell response includes a response that is, for example, 1%, 5%, 10%, 20%, 30%, 40%, 50%, 60%, 70%, 80%, 90%, 100%, 150%, 200%, 300%, 400%, 500% or more above a control T cell response. A reduced T cell response includes a response that is, for example, 1%, 5%, 10%, 20%, 30%, 40%, 50%, 60%, 70%, 80%, 90%, 100%, 150%, 200%, 300%, 400%, 500% or more below a control T cell response.

In some embodiments, a second control T cell response is contemplated. In some embodiments, the second control T cell response is a negative control T cell response. Exemplary negative controls include, but are not limited to, a T cell response in a sample that has been contacted with a non-T cell-activating peptide (e.g., a peptide not recognized by T cells present in a sample from a child), such as a non-CD4⁺-T cell-activating peptide, or a T cell response in sample that has not been contacted with a T cell-activating peptide (e.g., contacting the sample with a saline solution containing no peptides), such as a CD4⁺ T cell-activating peptide. Such a second control T cell response can be measured using any of the methods above or any other appropriate methods. In some embodiments, the second control T cell response is a positive control T cell response. Exemplary positive controls include, but are not limited to, a T cell response in a sample that has been contacted with a mitogen (e.g., phytohaemagglutinin, concanavalin A, lipopolysaccharide, or pokeweed mitogen). Positive and/or negative controls may be used to determine that an assay, such as an ELISA or ELISpot assay, is not defective or contaminated.

### Challenge

In some embodiments, methods provided herein comprise a challenge or a sample obtained from a child before, during, or after a challenge. Generally, a challenge comprises administering to the child a composition comprising wheat, rye, or barley, or a peptide thereof (e.g., a composition comprising an wheat gliadin, a rye secalin, or a barley hordein, or a peptide thereof), in some form for a defined period of time in order to activate the immune system of the child, e.g., through activation of wheat-, rye- and/or barley-reactive T cells and/or mobilization of such T cells in the child. Methods of challenges, e.g., gluten challenges, are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth-an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Any such methods of challenge that are capable of activating the immune system of the child, e.g., by activating wheat-, rye- or barley-reactive T-cells and and/or mobilizing such T cells into blood are contemplated herein.

In some embodiments, the challenge comprises administering a composition comprising wheat, barley and/or rye, or a peptide thereof. In some embodiments, the wheat is wheat flour, the barely is barley flour, and the rye is rye flour. In some embodiments, the challenge comprises administering a composition comprising a wheat gliadin, a barley hordein and/or a rye secalin, or a peptide thereof, to the child prior to determining a T cell response as described herein.

In some embodiments, the composition is administered to the child more than once prior to determining the T cell response, and a sample is obtained from the child after administration of the composition. In some embodiments, administration is daily for 3 days. In some embodiments, the sample is obtained from the child 6 days after administration of the composition. In some embodiments, the child has been on a gluten-free diet for at least 4 weeks prior to commencing the challenge.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a child before, during, and/or after a challenge as described herein. In some embodiments, the sample is a sample comprising a T cell, e.g., a whole blood sample or PBMCs. In some embodiments, the sample is contacted with a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the sample is contacted with a peptide as described herein, e.g., at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, a T cell response in the sample is measured as described herein.

### Treatment

Other aspects of the disclosure relate to treatment of children having or at risk of having Celiac disease. In some embodiments, the child to be treated is one identified as having or at risk of having Celiac disease by a method described herein, e.g., by evaluating a T cell response. In some embodiments, the methods comprise a step where information regarding treatment is provided to the child or the child's caregiver. A child's caregiver is any subject that is responsible for the care of the child. Examples of a child's caregiver include, but are not limited to, a parent, a step-parent, an adoptive parent, a foster parent or a guardian such as a grandparent, an aunt, an uncle, a sibling, a cousin, or a subject appointed by law or custom to care for the child. In some embodiments, the child's caregiver is an adult that is at least 18 years old. Such information can be given orally or in written form, such as 2 5 with written materials. Written materials may be in an electronic form. Any known treatment of Celiac disease is contemplated herein. Exemplary treatments include, e.g., a gluten-free diet. Other exemplary treatments include endopeptidases, such as ALV003 (Alvine) and AT1001 (Alba), agents that inhibit transglutaminase activity, agents that block peptide presentation by HLA DQ2.5, or oral resins that bind to gluten peptides and reduce their bioavailability.

In some embodiments, a method of treatment comprises administering an effective amount of a composition comprising a peptide comprising an adult immunodominant epitope, such as at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), to a child having or at risk of having Celiac disease. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 7); the second peptide comprises the amino acid sequence QPFPQPEQPFPWQP (SEQ ID NO: 8); and/or the third peptide comprises the amino acid sequence PEQPIPEQPQPYPQQ (SEQ ID NO: 9). Modifications to such peptides, e.g., an N-terminal pyro-glutamate and/or C-terminal amide, are contemplated and described herein. In some embodiments, the first peptide comprises the amino acid sequence ELQPFPQPELPYPQPQ (SEQ ID NO: 10), wherein the N-terminal glutamate is a pyroglutamate and the C-terminal glutamine is amidated (e.g., the free C-terminal COO is amidated); the second peptide comprises the amino acid sequence EQPFPQPEQPFPWQP (SEQ ID NO: 11), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 12), wherein the N-terminal glutamate is a pyroglutamate and the C-terminal glutamine is amidated (e.g., the free C-terminal COO is amidated). In some embodiments, the amino acid sequence of the first peptide is ELQPFPQPELPYPQPQ (SEQ ID NO: 10), wherein the N-terminal glutamate is a pyroglutamate and the C-terminal glutamine is amidated (e.g., the free C-terminal COO is amidated); the amino acid sequence of the second peptide is EQPFPQPEQPFPWQP (SEQ ID NO: 11), wherein the N-terminal glutamate is a pyroglutamate and the C-terminal proline is amidated (e.g., the free C-terminal COO is amidated); and/or the amino acid sequence of the third peptide is EPEQPIPEQPQPYPQQ (SEQ ID NO: 12), wherein the N-terminal glutamate is a pyroglutamate and the C-terminal glutamine is amidated (e.g., the free C-terminal COO is amidated).

Treatments may be administrated using any method known in the art. Pharmaceutical compositions suitable for each administration route are well known in the art (see, e.g., Remington: The Science and Practice of Pharmacy, 21st Ed. Lippincott Williams & Wilkins, 2005). In some embodiments, a treatment, e.g., a composition described herein, is administered via intradermal injection.

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Pharmaceutical compositions may include a pharmaceutically acceptable carrier. The term “pharmaceutically acceptable carrier” refers to molecular entities and compositions that do not produce an allergic, toxic or otherwise adverse reaction when administered to a child, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical composition include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington: The Science and Practice of Pharmacy, 21st Ed. Lippincott Williams & Wilkins, 2005).

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection. Examples of appropriate delivery mechanisms for intradermal administration include, but are not limited to, implants, depots, syringes, needles, capsules, and osmotic pumps.

It is especially advantageous to formulate the active agent in a dosage unit form for ease of administration and uniformity of dosage. “Dosage unit form” as used herein refers to physically discrete units suited as unitary dosages for the child to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of children. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the child to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the child, the site of delivery, the method of administration and other factors known to practitioners. Examples of techniques and protocols can be found in, e.g., Remington: The Science and Practice of Pharmacy, 21st Ed. Lippincott Williams & Wilkins, 2005. Effective amounts may be measured from ng/kg body weight to g/kg body weight per minute, hour, day, week or month. Dosage amounts may vary from, e.g., 10 ng/kg to up to 100 mg/kg of mammal body weight or more per day, preferably about 1 μg/kg/day to 10 mg/kg/day, depending upon the route of administration. In some embodiments, the effective amount is 150 micrograms of the peptides provided herein (i.e., 50 micrograms of the first peptide and an equimolar amount of each of the second and third peptides). In some embodiments, the effective amount is 26.5 nmol of each of the first, second, and third peptides. In some embodiments, the effective amount is 75 micrograms of the peptides provided herein (i.e., 25 micrograms of the first peptide and an equimolar amount of each of the second and third peptides). In some embodiments, the effective amount is 13.2 nmol of each of the first, second, and third peptides. Methods for producing equimolar peptide compositions are known in the art and provided herein (see, e.g., Example 3 and Muller et al. Successful immunotherapy with T-cell epitope peptides of bee venom phospholipase A2 induces specific T-cell anergy in patient allergic to bee venom. J. Allergy Clin. Immunol. Vol. 101, Number 6, Part 1: 747-754 (1998)). In some embodiments, this effective amount of the peptides is administered in sterile sodium chloride 0.9% USP as a bolus intradermal injection. In some embodiments, the first, second and third peptides or the composition are/is administered for eight weeks. In some embodiments, the first, second and third peptides or the composition are/is administered to the child in two phases. In some embodiments, the first phase is administration of the first, second and third peptides or the composition at an effective amount of 75 micrograms or 150 micrograms and the second phase is administration of the is administration of the first, second and third peptides or the composition at an effective amount of 150 micrograms. In some embodiments, the first phase comprises administration of the first, second and third peptides or the composition to the child at an effective amount of 75 micrograms or 150 micrograms twice weekly, for eight weeks and the second phase comprises administration of the first, second and third peptides or the composition to the child at an effective amount of 150 micrograms. In some embodiments, an assessment is performed between the first and second phase, e.g., a T cell response assay as described herein.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the child and/or induction of suppressor T cell subpopulations to secrete cytokines. Thus, a child treated according to the disclosure, in some embodiments, preferably is able to eat at least wheat, rye, and barley without a significant T cell response which would normally lead to symptoms of Celiac disease. In some embodiments, an effective amount of a treatment is administered. The term “effective amount” means the amount of a treatment sufficient to provide the desired therapeutic or physiological effect when administered under appropriate or sufficient conditions.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Children

Compositions and methods described herein are for use with a subject who is a child that is suspected of having or having Celiac disease. Preferably, the child is a human child. In some embodiments, the child is between 3 and 17 years of age. In some embodiments, the child is between 3 and 10 years of age.

In some embodiments, the child has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments, the child is HLA-DQ2.5 positive (i.e., has both susceptibility alleles DQA1*05 and DQB1*02). In some embodiments, the child may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes.

In some embodiments, the child is on a gluten-free diet.

### Samples

Samples, as used herein, refer to biological samples taken or derived from a child, e.g., a child having or suspected of having Celiac disease. Examples of samples include tissue samples or fluid samples. Examples of fluid samples are whole blood, plasma, serum, and other bodily fluids that comprise T cells. In some embodiments, the sample comprises T cells. In some embodiments, the sample comprises T cells and monocytes and/or granulocytes. In some embodiments, the sample comprising T cells comprise whole blood or peripheral blood mononuclear cells (PBMCs). The T cell may be a CD4+ T cell, e.g., a gluten-reactive CD4+ T cell. In some embodiments, the methods described herein comprise obtaining or providing the sample. In some embodiments, a first sample and second sample are contemplated. In some embodiments, the first sample is obtained from a child before administration of a composition comprising a peptide comprising an adult immunodominant epitope, such as at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), or a challenge described herein. In some embodiments, the second sample is obtained from a child after administration of the composition or after a challenge described herein. Additional samples, e.g., third, fourth, fifth, etc., are also contemplated if additional measurements of a T cell response are desired. Such additional samples may be obtained from the child at any time, e.g., before or after administration of a composition comprising a peptide comprising an adult immunodominant epitope, such as one comprising at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or a third peptide comprising the amino acid sequence PIPEQPQPY (SEQ ID NO: 5) and EQPIPEQPQ (SEQ ID NO: 6), or a challenge described herein.

### Controls and Control Subjects

In some embodiments, methods provided herein comprise measuring or use of a control T cell response. In some embodiments, the control T cell response is a T cell response in a sample from the child, e.g., before or during a challenge as described herein.

In some embodiments, the control T cell response is a T cell response in a sample obtained from a control subject (or subjects). In some embodiments, a control subject, e.g., a control child, has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject, e.g., a control child, does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject, e.g., a control child, is a healthy individual not having or suspected of having Celiac disease. In some embodiments, a control subject is an adult. In some embodiments, a control subject is a child. In some embodiments, control subjects are a population of adults, a population of children, or a population containing both adults and children. In some embodiments, a control level is a pre-determined value from a control subject or subjects, such that the control level need not be measured every time the methods described herein are performed.

### Peptides and Compositions Comprising Peptides

Aspects of the disclosure relate to use of peptides and compositions comprising peptides for identifying and/or treating a child having or suspected of having Celiac disease. These peptides comprise at least one adult immunodominant epitope. An adult immunodominant epitope is an amino acid sequence or motif that causes a T cell response that contributes to Celiac disease in an adult or a population of adults. In some embodiments, an adult is a subject that is at least 18 years old. In some embodiments, an adult immunodominant epitope causes a significant amount or majority of the T cell response against gluten in an adult or a population of adults.

In some embodiments, the peptide comprises at least one epitope selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), (SEQ ID NO: 5) and EQPIPEQPQ (SEQ ID NO: 6). In some embodiments, the peptide is at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the peptide is at least one of (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). In some embodiments, the first peptide comprises LQPFPQPELPYPQPQ (SEQ ID NO: 7); the second peptide comprises QPFPQPEQPFPWQP (SEQ ID NO: 8); and/or the third peptide comprises PEQPIPEQPQPYPQQ (SEQ ID NO: 9).

The length of the peptide may vary. In some embodiments, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments, peptides are, e.g., 4-1000, 4-500, 4-100, 4-50, 4-40, 4-30, or 4-20 amino acids in length. In some embodiments, peptides are 4-20, 5-20, 6-20, 7-20, 8-20, 9-20, 10-20, 11-20, 12-20, 13-20, 14-20, or 15-20 amino acids in length. In some embodiments, peptides are e.g., 5-30, 10-30, 15-30 or 20-30 amino acids in length. In some embodiments, peptides are 4-50, 5-50, 6-50, 7-50, 8-50, 9-50, 10-50, 11-50, 12-50, 13-50, 14-50, or 15-50 amino acids in length. In some embodiments, peptides are 8-30 amino acids in length.

In some embodiments, one or more glutamate residues of a peptide may be generated by tissue transglutaminase (tTG) deamidation activity upon one or more glutamine residues of the peptide. This deamidation of glutamine to glutamate causes the generation of peptides that can bind to HLA-DQ2 or -DQ8 molecules with high affinity. This reaction may occur in vitro by contacting the peptide composition with tTG outside of the child (e.g., prior to or during contact of a peptide composition with a sample comprising T cells from a child) or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 13) could become PFPQPELPY (SEQ ID NO: 1) after processing by tTG. Conservative substitution of E with D is also contemplated herein (e.g., PFPQPELPY (SEQ ID NO: 1) could become PFPQPDLPY (SEQ ID NO: 14). Exemplary peptides including an E to D substitution include peptide comprising or consisting of PFPQPDLPY (SEQ ID NO: 15), PQPDLPYPQ (SEQ ID NO: 16), PFPQPDQPF (SEQ ID NO: 17), PQPDQPFPW (SEQ ID NO: 18), PIPDQPQPY (SEQ ID NO: 19), LQPFPQPDLPYPQPQ (SEQ ID NO: 20), QPFPQPDQPFPWQP (SEQ ID NO: 21), or PQQPIPDQPQPYPQQ (SEQ ID NO: 22). Such substituted peptides can be the peptides of any of the methods and compositions provided herein.

A peptide may also be an analog of any of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any of the peptides provided, such variants can include conservative amino acid substitution variants, e.g., E to D substitution.

In some embodiments, analogs may include one or more amino acid substitutions as shown in Table 1 (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference). The peptides provided herein include analogs of SEQ ID NO: 23 comprising one or more of the listed amino acid substitutions. In some embodiments, the analog is an analog of SEQ ID NO: 23 comprising one of the amino acid substitutions provided in Table 1 below.

TABLE 1
Exemplary substitutions in the epitope FPQPELPYP (SEQ ID NO: 23)

| Amino acid in | | | | | | | | | |
|---|---|---|---|---|---|---|---|---|---|
| epitope | F | P | Q | P | E | L | P | Y | P |
| Exemplary | A, G, H, I, | A, F, I, M, | A, F, G, | — | D | M | S | I, S, | S, T, Y |
| Substitutions | L, M P, S, | S, T, V, | H, I, L, | | | | | V, W | |
| | T, W, Y | W, Y | M, S, T, V | | | | | | |

In some embodiments, a composition comprising at least one or one or more peptide(s) is contemplated. In some embodiments, the methods described herein comprise administering the composition to a child (e.g., a child having or suspected of having Celiac disease). In some embodiments, the composition is formulated for intradermal administration to a child. In some embodiments, the composition is formulated as a bolus for intradermal injection to a child. In some embodiments, the composition is formulated as a sterile, injectable solution. In some embodiments, the sterile, injectable solution is sodium chloride. In some embodiments, the sodium chloride is sterile sodium chloride 0.9% USP.

In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 6). “First”, “second”, and “third” are not meant to imply an order of use or importance, unless specifically stated otherwise. In some embodiments, the peptides are 8-30 amino acids in length. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 7); the second peptide comprises QPFPQPEQPFPWQP (SEQ ID NO: 8); and/or the third peptide comprises PEQPIPEQPQPYPQQ (SEQ ID NO: 9).

In some embodiments, it may be desirable to utilize the non-deamidated forms of such peptides, e.g., if the peptides are contained within a composition for administration to a child where tissue transglutaminase will act in situ (see, e.g., Øyvind Molberg et al. T cells from celiac disease lesions recognize gliadin epitopes deamidated in situ by endogenous tissue transglutaminase. Eur. J. Immunol. 2001. 31: 1317-1323). Accordingly, in some embodiments, the composition comprises at least one of: (i) a first peptide comprising the amino acid sequence PFPQPQLPY (SEQ ID NO: 13) and PQPQLPYPQ (SEQ ID NO: 24), (ii) a second peptide comprising the amino acid sequence PFPQPQQPF (SEQ ID NO: 25) and PQPQQPFPW (SEQ ID NO: 26), and (iii) a third peptide comprising the amino acid sequence PIPQQPQPY (SEQ ID NO: 27). In some embodiments, the first peptide comprises LQPFPQPQLPYPQPQ (SEQ ID NO: 28); the second peptide comprises QPFPQPQQPFPWQP (SEQ ID NO: 29); and/or the third peptide comprises PQQPIPQQPQPYPQQ (SEQ ID NO: 30). In some embodiments, the peptides are 8-30 amino acids in length.

Modifications to a peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A peptide may also be chemically modified at the level of amino acid side chains, of amino acid chirality, and/or of the peptide backbone.

Particular changes can be made to a peptide to improve resistance to degradation or optimize solubility properties or otherwise improve bioavailability compared to the parent peptide, thereby providing peptides having similar or improved therapeutic, diagnostic and/or pharmacokinetic properties. A preferred such modification includes the use of an N-terminal acetyl group or pyroglutamate and/or a C-terminal amide. Such modifications have been shown in the art to significantly increase the half -life and bioavailability of the peptides compared to the parent peptides having a free N- and C-terminus (see, e.g., PCT Publication No.: WO/2010/060155). In some embodiments, a peptide comprises an N-terminal acetyl group or pyroglutamate group, and/or a C-terminal amide group. In some embodiments, the first, second and/or third peptides described above comprise an N-terminal acetyl group or pyroglutamate group, and/or a C-terminal amide group. In some embodiments, the first peptide comprises ELQPFPQPELPYPQPQ (SEQ ID NO: 10), wherein the N-terminal E is a pyroglutamate; the second peptide comprises EQPFPQPEQPFPWQP (SEQ ID NO: 11), wherein the N-terminal E is a pyroglutamate; and/or the third peptide comprises EPEQPIPEQPQPYPQQ (SEQ ID NO: 12), wherein the N-terminal E is a pyroglutamate. In some embodiments, the first peptide comprises the amino acid sequence ELQPFPQPELPYPQPQ (SEQ ID NO: 10), wherein the N-terminal glutamate is a pyroglutamate and the C-terminal glutamine is amidated (e.g., the free C-terminal COO is amidated); the second peptide comprises the amino acid sequence EQPFPQPEQPFPWQP (SEQ ID NO: 11), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 12), wherein the N-terminal glutamate is a pyroglutamate and the C-terminal glutamine is amidated (e.g., the free C-terminal COO is amidated). In some embodiments, the first peptide consists of the amino acid sequence ELQPFPQPELPYPQPQ (SEQ ID NO: 10), wherein the N-terminal glutamate is a pyroglutamate and the C-terminal glutamine is amidated (e.g., the free C-terminal COO is amidated); the second peptide consists of the amino acid sequence EQPFPQPEQPFPWQP (SEQ ID NO: 11), wherein the N-terminal glutamate is a pyroglutamate and the C-terminal proline is amidated (e.g., the free C-terminal COO is amidated); and/or the third peptide consists of the amino acid sequence EPEQPIPEQPQPYPQQ (SEQ ID NO: 12), wherein the N-terminal glutamate is a pyroglutamate and the C-terminal glutamine is amidated (e.g., the free C-terminal COO is amidated).

### Peptide Production

The peptides can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides.

For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides or proteins, especially from food extracts. For example, a longer peptide or protein may be contacted with an enzyme that degrades the longer peptide or protein into shorter peptide fragments.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

### Other Testing

In some embodiments, methods described herein comprise other testing of a child (e.g., based on the results of the methods described herein). As used herein, “other testing” describes use of at least one additional diagnostic method in addition to the methods provided herein. Any diagnostic method or combinations thereof for Celiac disease is contemplated as other testing. Exemplary other testing includes, but is not limited to, intestinal biopsy, serology (measuring the levels of one or more antibodies present in the serum), genotyping (see, e.g., Walker-Smith J A, et al. Arch Dis Child 1990), and measurement of a T cell response. Such other testing may be performed as part of the methods described herein or after the methods described herein (e.g., as a companion diagnostic), or before use of the methods described herein (e.g., as a first-pass screen to eliminate certain children before use of the methods described herein, e.g., eliminating those that do not have one or more HLA-DQA and HLA-DQB susceptibility alleles).

When performing intestinal biopsies, generally multiple biopsies are taken from the second or third part of the duodenum. Endoscopy has become the most convenient method of obtaining biopsies of the small-intestinal mucosa. Suction biopsy (with a Crosby capsule) can provide the best samples. Celiac disease (CD) affects the mucosa of the proximal small intestine, with damage gradually decreasing in severity towards the distal small intestine, although in severe cases the lesions can extend to the ileum. The degree of proximal damage varies greatly depending on the severity of the disease. The proximal damage may be very mild in “silent” cases, with little or no abnormality detectable histologically in the mid-jejunum. Abnormalities in the gastric and rectal mucosa may be observed in some cases. Occasionally, the lesion in the duodenum/upper jejunum can be patchy, which may justify a second biopsy immediately in selected patients with positive endomysial antibody (EMA). However, this is only warranted if all three samples of the first biopsy show a normal histology.

Detection of serum antibodies (serology) is also contemplated. The presence of such serum antibodies can be detected using methods known to those of skill in the art, e.g., by ELISA, histology, cytology, immunofluorescence or western blotting. Such antibodies include, but are not limited to: IgA ant-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Children can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRBS & DQB1 by PCR with 144 primer mixes utilizing sequence-specific primers (PCR-SSP). Tissue Antigens 46, 355-367 (1995); Olerup 0, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Children that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, M. Green and J. Sambrook, Molecular Cloning: A Laboratory Manual (Fourth Edition), Cold Spring Harbour Laboratory Press (2012); T. Brown, Essential Molecular Biology:A Practical Approach Volumes I and II, Oxford University Press (2000); T. Brown, DNA Cloning: An Introduction, Wiley-Blackwell, 6^{th} edition (2010); F. M. Ausubel et al. (editors), Current Protocols in Molecular Biology, Wiley Online Library (Current Edition); Ed Harlow and David Lane (editors) Antibodies: A Laboratory Manual, Cold Spring Harbour Laboratory, 2 Lab edition (2013); and J. E. Coligan et al. (editors), Current Protocols in Immunology, Wiley Online Library (Current Edition).

In any one aspect or embodiment provided herein “comprising” may be replaced with “consisting essentially of” or “consisting of”.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Methods

An oral wheat bread challenge was conducted in DQ2.5+ children and adolescents having Celiac disease. The age ranges were 3-5, 6-10 and 11-18 years of age. An ELISpot peripheral blood mononucleated cell (PBMC) assay was performed to measure the level of IFNγ released by the PBMCs after being contacted with peptide 1, peptide 2, peptide 3, or a combination thereof. The identities of peptides 1, 2, and 3 are shown below.

Peptide 1:
(SEQ ID NO: 10)
ELQPFPQPELPYPQPQ
Peptide 2:
(SEQ ID NO: 11)
EQPFPQPEQPFPWQP
Peptide 3:
(SEQ ID NO: 12)
EPEQPIPEQPQPYPQQ

For each of peptide 1, 2, and 3, the N-terminal glutamate was a pyroglutamate and the carboxyl group of the C-terminal proline or glutamine was amidated. Peptide 1 comprises the T cell epitopes PFPQPELPY (SEQ ID NO: 1) and PQPELPYPQ (SEQ ID NO: 2). Peptide 2 comprises the T cell epitope PFPQPEQPF (SEQ ID NO: 3) and PQPEQPFPW (SEQ ID NO: 4). Peptide 3 comprises the T cell epitope PIPEQPQPY (SEQ ID NO: 5) and EQPIPEQPQ (SEQ ID NO: 6). These epitopes were previously identified as being dominant T cell epitopes in adults.

### Results

Tables 2-5 show that peptides 1, 2, and 3, and combinations thereof, were able to 2 0 induce a T cell response in PBMC samples from children after gluten challenge as indicated by the general increase in SFU in each child.

TABLE 2

| | Subject ID | | | | | | | |
|---|---|---|---|---|---|---|---|---|
| | A | B | C | D | E | F | G | H |
| Age of Subject | 14 | 11 | 17 | 17 | 11 | 17 | 16 | 11 |
| Cells per Well | 5.0 × | 5.0 × | 5.0 × | 4.0 × | 5.0 × | 5.0 × | 4.0 × | 3.5 × |
| | 10\hat{ }5 | 10\hat{ }5 | 10\hat{ }5 | 10\hat{ }5 | 10\hat{ }5 | 10\hat{ }5 | 10\hat{ }5 | 10\hat{ }5 |
| Blank | 2 | 0 | 1 | 1 | 1 | 1 | 1 | 1 |
| D-Gli 25 | 6 | 4 | 12 | 23 | 30 | 55 | 5 | 13 |
| D-Gli 25 | 4 | 1 | 8 | 17 | 38 | 62 | 1 | 6 |
| D-Gli 50 | 3 | 6 | 15 | 43 | 84 | 75 | 7 | 14 |
| D-Gli 50 | 6 | 2 | 19 | 40 | 64 | 110 | 4 | 18 |
| D-Gli 100 | 8 | 5 | 25 | 42 | 83 | 141 | 8 | 29 |
| D-Gli 100 | 12 | 6 | 25 | 47 | 74 | 134 | 7 | 26 |
| WT-Gli 25 | 3 | 1 | 4 | 21 | 7 | 27 | 0 | 4 |
| WT-Gli 25 | 3 | 1 | 8 | 15 | 4 | 40 | 2 | 3 |
| WT-Gli 50 | 7 | 0 | 14 | 31 | 8 | 38 | 4 | 8 |
| WT-Gli 50 | 4 | 0 | 9 | 34 | 13 | 26 | 2 | 8 |
| WT-Gli 100 | 7 | 2 | 28 | 36 | 8 | 47 | 9 | 15 |
| WT-Gli 100 | 5 | 4 | 16 | 41 | 17 | 67 | 8 | 17 |
| D-Glut 25 | 2 | 2 | 10 | 20 | 48 | 91 | 1 | 2 |
| D-Glut 25 | 3 | 2 | 9 | 13 | 37 | 68 | 4 | 3 |
| D-Glut 50 | 4 | 3 | 5 | 26 | 45 | 64 | 1 | 8 |
| D-Glut 50 | 5 | 1 | 7 | 19 | 58 | 78 | 6 | 3 |
| D-Glut 100 | 10 | 2 | 11 | 33 | 38 | 97 | 3 | 15 |
| D-Glut 100 | 6 | 3 | 11 | 41 | 64 | 156 | 3 | 10 |
| WT-Glut 25 | 2 | 3 | 8 | 3 | 0 | 35 | 3 | 6 |
| WT-Glut 25 | 2 | 1 | 8 | 8 | 2 | 75 | 1 | 2 |
| WT-Glut 50 | 4 | 2 | 10 | 24 | 3 | 61 | 2 | 1 |
| WT-Glut 50 | 3 | 3 | 5 | 39 | 2 | 37 | 3 | 1 |
| WT-Glut 100 | 7 | 4 | 11 | 30 | 11 | 69 | 4 | 6 |
| WT-Glut 100 | 3 | 0 | 14 | 21 | 6 | 51 | 1 | 6 |
| Pept-1 25 | 6 | 4 | 10 | 28 | 177 | 151 | 8 | 14 |
| Pept-1 25 | 2 | 3 | 5 | 32 | 139 | 146 | 14 | 13 |
| Pept-1 50 | 4 | 1 | 6 | 23 | 160 | 186 | 10 | 8 |
| Pept-1 50 | 1 | 5 | 5 | 31 | 144 | 147 | 10 | 8 |
| Pept-1 100 | 1 | 3 | 10 | 32 | 139 | 166 | 11 | 9 |
| Pept-1 100 | 4 | 2 | 2 | 25 | 153 | 138 | 5 | 8 |
| Pept-2 25 | 1 | 4 | 2 | 17 | 55 | 44 | 7 | 2 |
| Pept-2 25 | 6 | 6 | 5 | 30 | 45 | 59 | 7 | 7 |
| Pept-2 50 | 4 | 7 | 4 | 29 | 45 | 64 | 11 | 5 |
| Pept-2 50 | 3 | 8 | 4 | 32 | 43 | 105 | 5 | 12 |
| Pept-2 100 | 5 | 5 | 7 | 18 | 67 | 41 | 5 | 3 |
| Pept-2 100 | 3 | 11 | 1 | 37 | 44 | 59 | 9 | 9 |
| Pept-3 25 | 1 | 5 | 8 | 2 | 3 | 3 | 0 | 1 |
| Pept-3 25 | 4 | 1 | 0 | 4 | 3 | 1 | 0 | 2 |
| Pept-3 50 | 0 | 1 | 3 | 2 | 6 | 2 | 0 | 2 |
| Pept-3 50 | 0 | 3 | 3 | 2 | 6 | 0 | 1 | 4 |
| Pept-3 100 | 0 | 3 | 5 | 2 | 3 | 0 | 1 | 0 |
| Pept-3 100 | 3 | 1 | 0 | 4 | 4 | 4 | 3 | 3 |
| Pept-1/2/3 10 | 5 | 12 | 7 | 54 | 146 | 177 | 14 | 6 |
| Pept-1/2/3 25 | 9 | 8 | 6 | 43 | 181 | 179 | 21 | 13 |
| Pept-1/2/3 50 | 4 | 7 | 11 | 56 | 159 | 172 | 8 | 9 |
| Pept-1/2/3 75 | 7 | 5 | 9 | 48 | 137 | 170 | 13 | 12 |
| Pept-1/2/3 100 | 3 | 8 | 6 | 45 | 100 | 180 | 14 | 10 |
| Pept-1/2/3 150 | 10 | 9 | 8 | 60 | 104 | 149 | 6 | 16 |

The values the rows starting from “Blank (SFU)” and ending at “Pept-1/2/3 (150)” are all spot forming unit (SFU) values. The values in the parentheses in the first column are the concentration of each peptide in micrograms that were added to the PBMCs (e.g., Pept-1 50=50 micrograms of Peptide 1, Pept-1/2/3 50=50 micrograms of each of Peptide 1, Peptide 2, and Peptide 3). Pept-1=Peptide 1, Pept-2=Peptide 2, Pept-3=Peptide 3, the amino acid identities of which are mentioned above. D-Gli=deamidated gliadin. WT-Gli=wild-type gliadin. D-Glut=deamidated gluten. WT-Glut=wild-type gluten.

TABLE 3

| | Subject ID | | | | | |
|---|---|---|---|---|---|---|
| | I | J | K | L | M | N |
| Age of Subject | 8 | 6 | 10 | 4 | 4 | 3 |
| Cells per Well | 5.0 × | 5.0 × | 3.2 × | 3.5 × | 2.3 × | 2.4 × |
| | 10\hat{ }5 | 10\hat{ }5 | 10\hat{ }5 | 10\hat{ }5 | 10\hat{ }5 | 10\hat{ }5 |
| Blank (SFU) | 1 | 1 | 1 | 1 | 0 | 0 |
| D-Gli 25 | 6 | 39 | 7 | 13 | 42 | 6 |
| D-Gli 25 | 6 | 38 | 19 | 13 | 33 | 5 |
| D-Gli 50 | 19 | 48 | 33 | 18 | 62 | 9 |
| D-Gli 50 | 10 | 61 | 30 | 19 | 37 | 4 |
| D-Gli 100 | 15 | 64 | 37 | 22 | 49 | 9 |
| D-Gli 100 | 18 | 77 | 47 | 21 | 54 | 7 |
| WT-Gli 25 | 2 | 9 | 13 | 10 | 40 | 1 |
| WT-Gli 25 | 5 | 9 | 3 | 14 | 15 | 1 |
| WT-Gli 50 | 8 | 16 | 8 | 12 | 41 | 0 |
| WT-Gli 50 | 5 | 15 | 14 | 13 | 45 | 3 |
| WT-Gli 100 | 8 | 26 | 8 | 11 | 56 | 4 |
| WT-Gli 100 | 4 | 16 | 18 | 24 | 33 | 2 |
| D-Glut 25 | 3 | 30 | 13 | 8 | 39 | 4 |
| D-Glut 25 | 3 | 19 | 13 | 6 | 37 | 5 |
| D-Glut 50 | 4 | 49 | 6 | 15 | 50 | 0 |
| D-Glut 50 | 1 | 57 | 13 | 15 | 54 | 2 |
| D-Glut 100 | 6 | 58 | 22 | 19 | 93 | 5 |
| D-Glut 100 | 12 | 82 | 13 | 21 | 62 | 3 |
| WT-Glut 25 | 1 | 5 | 8 | 8 | 19 | 5 |
| WT-Glut 25 | 3 | 0 | 6 | 6 | 23 | 2 |
| WT-Glut 50 | 0 | 6 | 8 | 14 | 45 | 1 |
| WT-Glut 50 | 3 | 5 | 14 | 18 | 45 | 4 |
| WT-Glut 100 | 10 | 11 | 12 | 10 | 45 | 5 |
| WT-Glut 100 | 3 | 13 | 4 | 11 | 50 | 4 |
| Pept-1 25 | 6 | 103 | 33 | 10 | 34 | 4 |
| Pept-1 25 | 7 | 109 | 28 | 9 | 18 | 6 |
| Pept-1 50 | 7 | 107 | 28 | 11 | 32 | 2 |
| Pept-1 50 | 3 | 92 | 20 | 5 | 30 | 9 |
| Pept-1 100 | 6 | 109 | 24 | 14 | 45 | 5 |
| Pept-1 100 | 2 | 111 | 38 | 7 | 40 | 3 |
| Pept-2 25 | 7 | 85 | 17 | 5 | 28 | 2 |
| Pept-2 25 | 7 | 98 | 22 | 4 | 27 | 5 |
| Pept-2 50 | 5 | 107 | 19 | 6 | 26 | 4 |
| Pept-2 50 | 6 | 83 | 18 | 1 | 29 | 10 |
| Pept-2 100 | 9 | 81 | 23 | 2 | 32 | 4 |
| Pept-2 100 | 4 | 90 | 15 | 4 | 24 | 7 |
| Pept-3 25 | 1 | 4 | 2 | 1 | 0 | 0 |
| Pept-3 25 | 0 | 2 | 3 | 1 | 0 | 1 |
| Pept-3 50 | 1 | 2 | 2 | 2 | 1 | 2 |
| Pept-3 50 | 1 | 3 | 1 | 0 | 0 | 1 |
| Pept-3 100 | 2 | 1 | 3 | 2 | 0 | 0 |
| Pept-3 100 | 0 | 2 | 0 | 0 | 0 | 0 |
| Pept-1/2/3 10 | 9 | 162 | 51 | 11 | 40 | 4 |
| Pept-1/2/3 25 | 11 | 129 | 41 | 4 | 43 | 11 |
| Pept-1/2/3 50 | 7 | 158 | 60 | 16 | 34 | 8 |
| Pept-1/2/3 75 | 12 | 136 | 51 | 7 | 35 | 8 |
| Pept-1/2/3 100 | 0 | 136 | 1 | 7 | 37 | 9 |
| Pept-1/2/3 150 | 11 | 113 | 48 | 9 | 36 | 6 |

The values for the rows starting from “Blank (SFU)” and ending at “Pept-1/2/3 (150)” are all spot forming unit (SFU) values. The values in the parentheses in the first column are the concentration of each peptide in micrograms that were added to the PBMCs (e.g., Pept-1 50=50 micrograms of Peptide 1, Pept-1/2/3 50=50 micrograms of each of Peptide 1, Peptide 2, and Peptide 3). Pept-1=Peptide 1, Pept-2=Peptide 2, Pept-3=Peptide 3, the amino acid identities of which are mentioned above. D-Gli=deamidated gliadin. WT-Gli=wild-type gliadin. D-Glut=deamidated gluten. WT-Glut=wild-type gluten.

TABLE 4

| | Subject ID | | | | | |
|---|---|---|---|---|---|---|
| | P | Q | R | S | T | U |
| Age of Subject | 16 | 17 | 14 | 13 | 17 | 15 |
| Cells per Well | 3.0 × | 3.0 × | 3.0 × | 3.0 × | 3.0 × | 3.0 × |
| | 10\hat{ }5 | 10\hat{ }5 | 10\hat{ }5 | 10\hat{ }5 | 10\hat{ }5 | 10\hat{ }5 |
| Blank (SFU) | 1 | 0 | 16 | 2 | 1 | 3 |
| D-Gli 100 | 15 | 35 | 28 | 2 | 26 | 20 |
| D-Gli 100 | 13 | 65 | 47 | 9 | 20 | 14 |
| WT-Gli 100 | 29 | 43 | 28 | 0 | 7 | 8 |
| WT-Gli 100 | 27 | 37 | 40 | 4 | 18 | 11 |
| D-Glut 100 | 26 | 16 | 40 | 4 | 13 | 5 |
| D-Glut 100 | 49 | 10 | 26 | 1 | 3 | 8 |
| WT-Glut 100 | 26 | 54 | 34 | 2 | 54 | 34 |
| WT-Glut 100 | 13 | 59 | 23 | 1 | 42 | 19 |
| Pept-1 0.1 | 16 | 12 | 12 | 2 | 20 | 6 |
| Pept-1 0.1 | 15 | 18 | 16 | 4 | 18 | 7 |
| Pept-1 0.5 | 21 | 34 | 21 | 1 | 34 | 18 |
| Pept-1 0.5 | 24 | 32 | 26 | 2 | 31 | 20 |
| Pept-1 1 | 49 | 45 | 22 | 1 | 55 | 33 |
| Pept-1 1 | 31 | 36 | 21 | 2 | 60 | 24 |
| Pept-1 2.5 | 45 | 67 | 19 | 0 | 68 | 24 |
| Pept-1 2.5 | 36 | 56 | 19 | 3 | 44 | 24 |
| Pept-1 5 | 37 | 72 | 18 | 4 | 52 | 42 |
| Pept-1 5 | 32 | 70 | 17 | 3 | 51 | 35 |
| Pept-1 10 | 32 | 45 | 15 | 6 | 66 | 41 |
| Pept-1 10 | 59 | 41 | 21 | 4 | 43 | 50 |
| Pept-1 25 | 48 | 62 | 16 | 5 | 51 | 61 |
| Pept-1 25 | 32 | 90 | 10 | 1 | 63 | 54 |
| Pept-1 50 | 46 | 70 | 16 | 2 | 78 | 49 |
| Pept-1 50 | 37 | 65 | 9 | 3 | 55 | 41 |
| Pept-2 0.1 | 6 | 25 | 18 | 2 | 33 | 8 |
| Pept-2 0.1 | 14 | 12 | 20 | 0 | 47 | 6 |
| Pept-2 0.5 | 10 | 44 | 16 | 2 | 34 | 8 |
| Pept-2 0.5 | 8 | 51 | 14 | 1 | 39 | 12 |
| Pept-2 1 | 18 | 31 | 15 | 5 | 41 | 13 |
| Pept-2 1 | 11 | 41 | 11 | 3 | 51 | 15 |
| Pept-2 2.5 | 31 | 44 | 11 | 2 | 49 | 26 |
| Pept-2 2.5 | 11 | 51 | 33 | 3 | 56 | 33 |
| Pept-2 5 | 19 | 30 | 16 | 5 | 68 | 28 |
| Pept-2 5 | 15 | 38 | 14 | 4 | 77 | 21 |
| Pept-2 10 | 19 | 64 | 11 | 2 | 87 | 31 |
| Pept-2 10 | 14 | 60 | 14 | 2 | 74 | 25 |
| Pept-2 25 | 25 | 37 | 29 | 5 | 86 | 23 |
| Pept-2 25 | 37 | 60 | 31 | 2 | 65 | 13 |
| Pept-2 50 | 16 | 57 | 16 | 2 | 61 | 13 |
| Pept-2 50 | 24 | 69 | 17 | 2 | 74 | 21 |
| Pept-3 50 | 8 | 1 | 12 | 0 | 3 | 0 |
| Pept-3 50 | 6 | 2 | 9 | 1 | 1 | 2 |
| Pept-1/2/3 0.3 | 18 | 24 | 44 | 3 | 41 | 16 |
| Pept-1/2/3 1.5 | 20 | 66 | 15 | 2 | 65 | 33 |
| Pept-1/2/3 3 | 41 | 65 | 27 | 1 | 108 | 40 |
| Pept-1/2/3 15 | 45 | 74 | 20 | 4 | 81 | 45 |
| Pept-1/2/3 30 | 50 | 65 | 30 | 2 | 76 | 34 |
| Pept-1/2/3 150 | 38 | 79 | 17 | 0 | 80 | 34 |

The values the rows starting from “Blank (SFU)” and ending at “Pept-1/2/3 (150)” are all spot forming unit (SFU) values. The values in the parentheses in the first column are the concentration of each peptide in micrograms that were added to the PBMCs (e.g., Pept-1 50=50 micrograms of Peptide 1, Pept-1/2/3 50=50 micrograms of each of Peptide 1, Peptide 2, and Peptide 3). Pept-1=Peptide 1, Pept-2=Peptide 2, Pept-3=Peptide 3, the amino acid identities of which are mentioned above. D-Gli=deamidated gliadin. WT-Gli=wild-type gliadin. D-Glut=deamidated gluten. WT-Glut=wild-type gluten.

TABLE 5

| | Subject ID | | | |
|---|---|---|---|---|
| | V | W | X | Y |
| Age of subject | 10 | 10 | 7 | 6 |
| Cells per Well | 3.0 × 10\hat{ }5 | 3.0 × 10\hat{ }5 | 3.0 × 10\hat{ }5 | 2.29 × 10\hat{ }5 |
| Blank (SFU) | 3 | 10 | 3 | 2 |
| D-Gli 100 | 1 | 7 | 2 | 11 |
| D-Gli 100 | 8 | 15 | 1 | 20 |
| WT-Gli 100 | 5 | 18 | 1 | 18 |
| WT-Gli 100 | 11 | 15 | 2 | 15 |
| D-Glut 100 | 7 | 5 | 4 | 24 |
| D-Glut 100 | 3 | 7 | 1 | 28 |
| WT-Glut 100 | 13 | 19 | 1 | 16 |
| WT-Glut 100 | 14 | 14 | 0 | 19 |
| Pept-1 0.1 | 6 | 7 | 1 | 2 |
| Pept-1 0.1 | 5 | 8 | 0 | 6 |
| Pept-1 0.5 | 26 | 7 | 1 | 11 |
| Pept-1 0.5 | 8 | 8 | 0 | 11 |
| Pept-1 1 | 14 | 9 | 2 | 4 |
| Pept-1 1 | 20 | 15 | 5 | 7 |
| Pept-1 2.5 | 15 | 6 | 3 | 7 |
| Pept-1 2.5 | 15 | 12 | 1 | 8 |
| Pept-1 5 | 22 | 7 | 3 | 12 |
| Pept-1 5 | 18 | 8 | 4 | 12 |
| Pept-1 10 | 21 | 10 | 0 | 17 |
| Pept-1 10 | 24 | 5 | 3 | 18 |
| Pept-1 25 | 26 | 14 | 2 | 8 |
| Pept-1 25 | 14 | 3 | 0 | 6 |
| Pept-1 50 | 25 | 7 | 1 | 7 |
| Pept-1 50 | 35 | 6 | 0 | 14 |
| Pept-2 0.1 | 4 | 7 | 1 | 3 |
| Pept-2 0.1 | 13 | 2 | 1 | 5 |
| Pept-2 0.5 | 9 | 4 | 1 | 7 |
| Pept-2 0.5 | 12 | 5 | 1 | 11 |
| Pept-2 1 | 21 | 4 | 4 | 4 |
| Pept-2 1 | 14 | 5 | 2 | 15 |
| Pept-2 2.5 | 14 | 6 | 1 | 9 |
| Pept-2 2.5 | 21 | 6 | 5 | 11 |
| Pept-2 5 | 27 | 13 | 3 | 14 |
| Pept-2 5 | 22 | 3 | 5 | 12 |
| Pept-2 10 | 14 | 10 | 3 | 4 |
| Pept-2 10 | 15 | 10 | 1 | 8 |
| Pept-2 25 | 23 | 9 | 1 | 14 |
| Pept-2 25 | 29 | 5 | 1 | 13 |
| Pept-2 50 | 30 | 5 | 0 | 10 |
| Pept-2 50 | 20 | 13 | 0 | 10 |
| Pept-3 50 | 0 | 8 | 0 | 3 |
| Pept-3 50 | 2 | 8 | 2 | 3 |
| Pept-1/2/3 0.3 | 33 | 1 | 0 | 8 |
| Pept-1/2/3 1.5 | 31 | 12 | 2 | 13 |
| Pept-1/2/3 3 | 27 | 10 | 4 | 10 |
| Pept-1/2/3 15 | 37 | 10 | 2 | 21 |
| Pept-1/2/3 30 | 41 | 5 | 2 | 16 |
| Pept-1/2/3 150 | 17 | 11 | 0 | 11 |

The values the rows starting from “Blank (SFU)” and ending at “Pept-1/2/3 (150)” are all spot forming unit (SFU) values. The values in the parentheses in the first column are the concentration of each peptide in micrograms that were added to the PBMCs (e.g., Pept-1 50=50 micrograms of Peptide 1, Pept-1/2/3 50=50 micrograms of each of Peptide 1, Peptide 2, and Peptide 3). Pept-1=Peptide 1, Pept-2=Peptide 2, Pept-3=Peptide 3, the amino acid identities of which are mentioned above. D-Gli=deamidated gliadin. WT-Gli=wild-type gliadin. D-Glut=deamidated gluten. WT-Glut=wild-type gluten.

### Example 2

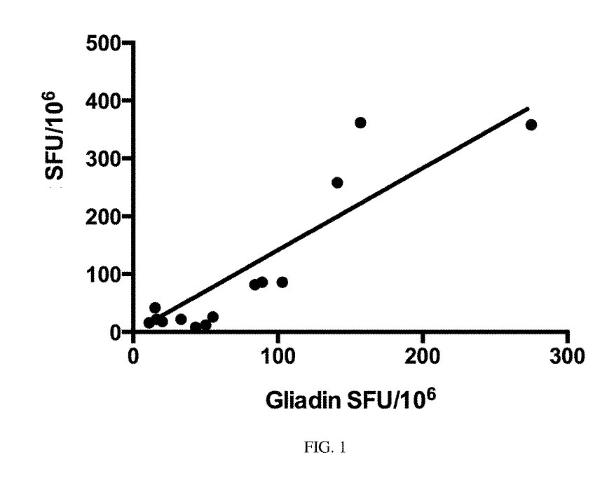

IFNγ ELISpot responses in 9 older children (11-17 yo) and 6 younger children (3-10 yo) were stimulated by the mixture of peptides 1, 2, and 3 described in Example 1 (each peptide at a concentration of 25 ug/mL) or stimulated by deamidated gliadin (100 ug/mL) after 3-day wheat challenge (r2=0.8, p<0.0001). The ELISpot responses were similar using either the peptide mixture of the deamidated gliadin (FIG. 1). This shows that the peptide mixture was capable of eliciting a T cell response similar to the deamidated gliadin. Peptide 1 and Peptide 2 were found to be the most active peptides in gliadin for children.

### Example 3

### Phase II-Pediatric Dose Ranging Study

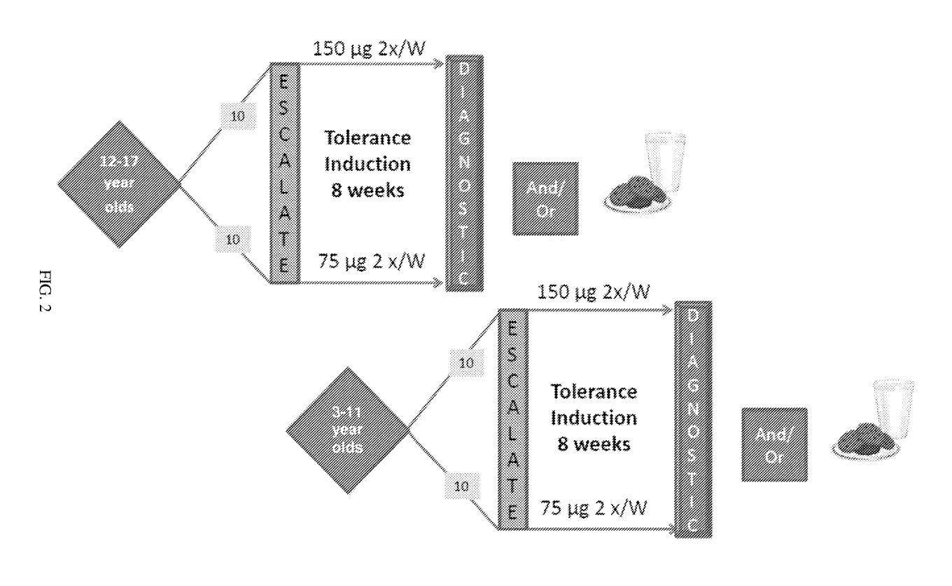

A dose-escalation study is performed in children (age ranges 12-17 years of age and 3-11 years of age). The study configuration is summarized in FIG. 2. Briefly, 75 micrograms or 150 micrograms of a peptide composition is injected intradermally into each child twice a week for up to 8 weeks. The peptide composition includes 3 peptides in sodium chloride 0.9% USP: ELQPFPQPELPYPQPQ (SEQ ID NO: 10), EQPFPQPEQPFPWQP (SEQ ID NO: 11), and EPEQPIPEQPQPYPQQ (SEQ ID NO: 12). For each peptide in the composition, the N-terminal glutamate is a pyroglutamate and the carboxyl group of the C-terminal proline or glutamine is amidated. After completion of the dosages, each child is then assessed using serology markers or given an oral gluten challenge to assess treatment efficacy. Further study details are provided below.
1) Objectives:
   To establish the dose in adolescents (12-17) first then establish dose in younger children (3-11) after safety assessment of adolescents.
2) Prior Study:
   Dose Escalation Study to establish a regimen that will minimize the risk of gastrointestinal adverse events.
   Phase 2 Adult Dose Ranging study.
3) Study that this will support: Phase 3 Efficacy trial in children using serology. Generally, essentially all children seroconvert in 90 days on gluten.
4) Key Inclusion/Exclusion: HLA DQ2+/DQ8-; Serology proven or biopsy proven Celiac disease; on a gluten free diet (GFD) for at least 1 year.
5) Key Assessments: Diagnostic test and/or 3 day oral challenge results after treatment regimen.

### Preparation of a 150 and a 75 Microgram Dosage Composition of the First, Second, and Third Peptide

A dose of 150 μg peptide composition is defined by there being 50 μg (26.5 nmol) of pure peptide 1, and an equimolar amount of peptide 2 and peptide 3. The molar equivalent of 50 μg peptide 1 is given by 50 μg/1889.3 g/mol=26.5 nmol. When preparing a solution containing 150 μg peptide composition for the constituent peptides, the weight of each peptide is adjusted according to peptide purity and peptide content of the lyophilized stock material. For example, if the peptide 1 stock material has peptide purity of 98% and its peptide content is 90%, the weight of stock material yielding 50 μig peptide 1 is 50 μg/(peptide purity×peptide content)=50 ug/(0.98×0.90)=56.7 ug.

The molar amount of peptide 2 in the peptide composition 150 μg is 26.5 nmol, and the weight of lyophilized peptide 2 stock material is therefore given by 26.5 nmol×1833.2 g/mol/(peptide purity x peptide content). For example, if peptide 2 peptide purity is 99%, and peptide content of 95%, the mass of stock required is 51.7 ug. The molar amount of peptide 3 in the peptide composition 150 ug is 26.5 nmol, and the weight of lyophilized peptide 3 stock material is therefore given by 26.5 nmol×1886.2 g/mol/(peptide purity x peptide content). For example, if peptide 3 peptide purity is 98%, and peptide content of 92%, the mass of stock required is 55.4 ug.

A dose of 75 μg peptide composition is defined by there being 25 μg (13.2 nmol) of pure peptide 1, and an equimolar amount of peptide 2 and peptide 3. The molar equivalent of 25 μg peptide 1 is given by 25 μg/1889.3 g/mol=13.2 nmol. When preparing a solution containing 75 μg peptide composition for the constituent peptides, the weight of each peptide is adjusted according to peptide purity and peptide content of the lyophilized stock material. For example, if the peptide 1 stock material has peptide purity of 98% and its peptide content is 90%, the weight of stock material yielding 25 μg peptide 1 is 25 μg/(peptide purity×peptide content)=25 ug/(0.98×0.90)=28.3 ug.

The molar amount of peptide 2 in the peptide composition 75 μg is 13.2 nmol, and the weight of lyophilized peptide 2 stock material is therefore given by 13.2 nmol×1833.2 g/mol/peptide purity×peptide content). For example, if peptide 2 peptide purity is 99%, and peptide content of 95%, the mass of stock required is 25.8 ug.

The molar amount of peptide 3 in peptide composition 75 ug is 13.2 nmol, and the weight of lyophilized peptide 3 stock material is therefore given by 26.5 nmol×1886.2 g/mol/(peptide purity×peptide content). For example, if peptide 3 peptide purity is 98%, and peptide content of 92%, the mass of stock required is 27.7 ug.

### Example 4

### Phase II-Pediatric Dose Ranging Study

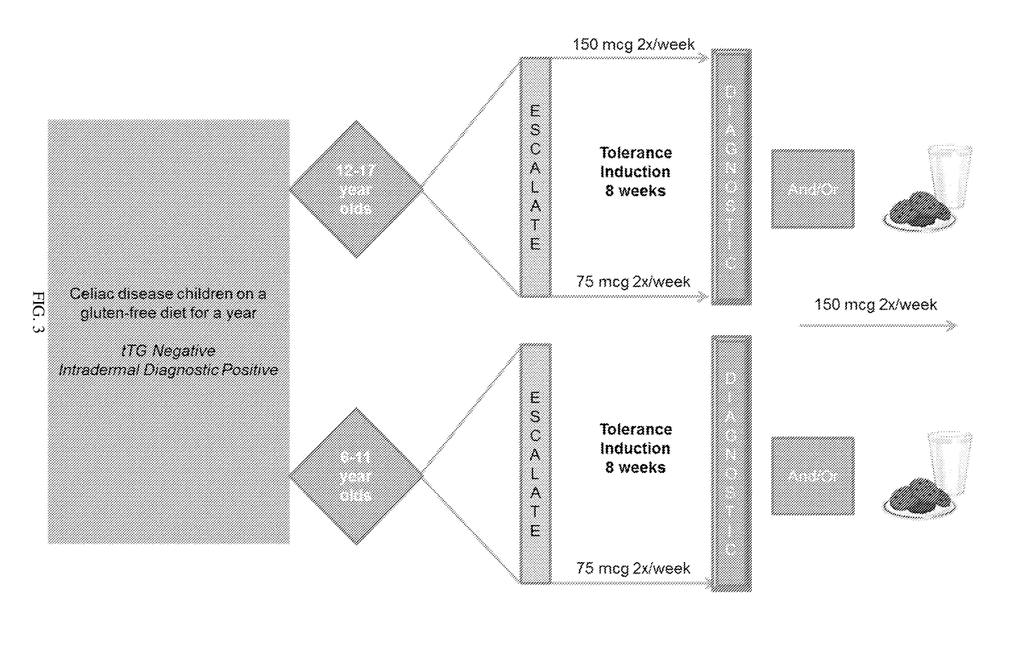

A dose-escalation study is performed in children (age ranges 12-17 years of age and 6-11 years of age). The children are on a gluten-free diet for at least one year and are tTG serology negative. The children also respond to intradermal injection of the peptide composition (e.g., by having increased levels of circulating cytokines after intradermal injection). The study configuration is summarized in FIG. 3. Briefly, 75 micrograms or 150 micrograms of a peptide composition is injected intradermally into each child twice a week for up to 8 weeks. The peptide composition is the same composition described in Example 3.

After completion of the dosages, each child is then assessed using serology markers or given an oral gluten challenge to assess treatment efficacy. Children may also be assessed using an ex vivo T cell diagnostic assay or an intradermal injection of the peptide composition (e.g., by assaying levels of circulating cytokines after intradermal injection). After assessment, children are further administered a dose of 150 micrograms injected intradermally into each child twice a week, e.g., until tolerance is achieved. Further study details are provided below.
1) Objective

To establish the dose in adolescents (12-17) first then establish dose in younger children (6-11) after safety assessment of adolescents
2) Prior Study Needed

Dose escalation study to establish a regimen that will minimize the risk of GI AEs

Phase 2 adult dose ranging study to determine if twice weekly dosing is needed during induction phase
3) Future Study Supported

Phase 3 efficacy trial in children using serology (2/3 seroconvert in 90 days)
4) Key Inclusion/Exclusion

HLA-DQ2.5+/DQ8-; serology proven celiac disease patients on a gluten-free diet for one year
5) Key Assessments

Diagnostic test and/or 3-day oral challenge results after tolerizing regimen

### Example 5

### The Specificity and Immunodominance of the Polyclonal T Cell Response to Gluten is Stable in Coeliac Disease Irrespective of Age

### Abstract

Characterising the gluten-specific immune response is critical for the development of novel antigen-specific approaches to diagnosis and treatment of coeliac disease (CD). Whilst well established in adults with CD, there is limited data from children with CD based on in vitro studies using long-term culture of T cell lines or proliferation assays. The aim herein was to characterise the in vivo T cell response following oral wheat gluten challenge in 3-17 year-olds with CD to wheat gluten peptides immunogenic to adults with CD. From 40 paediatric patients positive gluten-specific responses were detected in 30 individuals. Responses were to the same dominant peptides described for adults with CD, and deamidation enhanced the T cell response. Cross-reactivity was observed at both the polyclonal and clonal level, with wheat-specific T cells reacting to barley and rye peptides. It was observed that identical patterns of reactivity by T cell clones specific to alpha gliadin peptides being restricted, and omega gliadin T cell clones being highly promiscuous. Although age and time since diagnosis did not affect the T cell response, patients homozygous for HLA-DQ2.5 had a greater T cell response. For the first time herein it is shown in vivo that the specificity and flavour of the T cell response following ingestion of wheat in children with CD is consistent with that described for adults with CD. These findings have implications for the field of antigen-specific therapeutics, and suggest that peptide immunotherapies designed in adults can also be beneficial in children.

### Introduction

Coeliac disease (CD) is a prevalent systemic autoimmune illness characterised by a combination of dietary gluten-dependent clinical manifestations, CD-specific antibodies, and enteropathy [refs. 1,2]. Traditionally regarded a malabsorptive illness of childhood, the median age of diagnosis is now closer to 40 (Green) and the clinical presentation broad (Green). The development of CD is strongly dependent on the presence of the major histocompatibility complex encoded human leukocyte antigen (HLA) genes HLA-DQ2.5, HLA-DQ2.2, and/or HLA-DQ8, with HLA-DQ2.5 homozygosity a major determinant of CD development in childhood [refs. 3,4].

The strong association of CD with particular HLA genes underpins the central role for the activation of HLA-restricted gluten-specific CD4+ T cells in CD pathogenesis [ref. 5]. Optimal immunogenicity for the majority of gluten peptides is dependent on post-translational modification (deamidation) by transglutaminase, which converts site-selective glutamine residues to glutamate, enhancing binding to disease-associated HLA. There is a strong HLA-DQ2.5 gene dose effect; gluten presented by HLA-DQ2.5 homozygous antigen-presenting cells (APCs) results in at least a 4-fold higher T-cell response compared with gluten presentation by HLA-DQ2.5 heterozygous APCs [ref. 6]. Studies in adults (18 yrs+) with CD utilising three-day oral challenges with wheat, barley, and rye have revealed a hierarchy of gluten peptides derived from these cereals immunogenic in HLA-DQ2.5 associated CD in vivo [refs. 7-11]. Peptides derived from wheat gliadin encompassing the T cell epitopes DQ2.5-glia-α1/α2 and DQ2.5-glia-w1/w2 consistently (75-80% of CD adults) make a substantial contribution to the total gluten-reactive T cell population mobilised by oral wheat challenge. Importantly, many gluten peptides derived from wheat, barley, and rye prolamins are weak agonists in vitro for cross-reactive T cells specific for immunodominant epitopes [ref. 11]. In keeping with the role of dominant gluten peptides driving the immune response in CD, biased TCR gene usage has been described, with over-usage of the TRAV26-1 and TRBV7-2 gene segment in T cells specific for HLA-DQ2.5-glia-α2-specific and the conservation of a non-germline-encoded Arg residue in the CDR3b loop [refs. 12-14]. This biased TCR repertoire reflects in vivo antigen selection and the importance of deamidated gluten peptides.

In contrast to the comprehensive T cell epitope mapping in adults with CD there are limited studies on gluten-specific T cell responses in children (less than 18yrs) with CD, and most have relied upon PBMCs or T cell lines stimulated for prolonged periods in vitro. Vader et al. exploited gluten-specific intestinal T cell lines isolated from 16 children and 4 adults [ref. 15], and only half of the T-cell lines responded to DQ2.5-glia-α1a/αa2, and recognised six previously unreported epitopes half of which did not require deamidation for immunogenicity. The authors postulate that diversification of the immune response to epitope(s) distinct from the inciting antigen (epitope spreading) may account for the greater heterogeneity in gluten peptide responses identified in children, and that over time, the immune response focuses on immunodominant epitopes as a result of stronger binding affinity to specific deamidated peptides. Using in vitro proliferation assays to assess gluten and gluten peptides cultured with PBMC from children with untreated CD, two separate groups found poor or undetectable responses to DQ2.5-glia a1a and a2 [refs. 16,17].

Collectively, in vitro studies in children with CD indicate a lower rate of response to dominant T cell epitopes, a lower rate of dependence on deamidation, and novel immunogenic peptides. However it is unclear how much methodological issues, such as prolonged in vitro culture and use of potent mitogens, has contributed to these discrepant findings. For instance, we have shown in a single study employing T cells induced by in vivo gluten challenge after short-term oral challenge adolescents with CD (mean 18.6 years; range 15-24 yrs) recognise the 33 mer peptide encompassing DQ2.5-glia-α1a/α2/a3 [ref. 10], consistent with data on T cell responses in adults with CD.

A goal of autoimmune research is the development of antigen-specific applications targeting the specific causative antigenic peptides. These could be potentially used in diagnostics, preventative strategies or therapeutics that induce tolerance. However if the specificity of the immune response to gluten does change with prolonged antigen exposure, as suggested by Vader et al [ref. 15], this poses a significant challenge for the development of antigen-specific applications in autoimmune disease and allergy that will benefit both children and adults.

While it impossible to assess the primary T-cell response to gluten, oral gluten challenge and isolating T cells from blood allows the recall response against gluten to be readily compared between adults and children of any age. Although undertaking an unbiased, definitive study of gluten peptides recognised by T cells in children with CD is impractical due to the large volume of blood required to screen all potential epitopes, it is possible to test whether peptides immunodominant in adults are also important in the gluten-specific T cell response in children with CD. This study establishes the specificity and hierarchy of the polyclonal immune response to gluten in HLA-DQ2.5+ children with CD, and determines the redundancy of peptide recognition to enable definitive comparisons on the specificity, magnitude, maturity and clonality of T cell responses in children and adults.

### Material and Methods

### Subjects and Oral Grain Challenge

All participants or their parents provided written informed consent. All participants had biopsy-proven CD diagnosed according to ESPGHAN criteria [ref. 18], and possessed both alleles (HLA-DQA1*05 and HLA-DQB1*02 encoding the major CD-determining HLA-DQ haplotype (HLA-DQ2.5+) but did not possess either HLA-DQ allele encoding HLA-DQ8. Participants were required to have followed a strict gluten-free diet for at least the previous three months. The Australian cohort consisted of 40 paediatric CD patients (3-17; median 9.5; 16M:24F) split into three groups: 3-5, 6-10, and 11-18. An additional four adults (18+) with CD were recruited for comparison of T cells responses. See Table 6 for cohort details.

TABLE 6
Cohort details

| | | | | | | | T cell | |
|---|---|---|---|---|---|---|---|---|
| | | | | | | | response | |
| | | | | | Years | | (Low +, Int | Symptoms (Mild |
| | | | Challenge | DQ2.5 | since | Elevated | ++, High | +, Moderate ++, |
| ID | Age | Sex | completed | zygosity | diagnosis | serology | +++) | Severe +++) |
| M | 4 | F | Y | 2.5/2.5 | 1.5 | N | ++ | Asmptomatic |
| N | 3 | F | N | 2.5/x | 1.3 | N | + | Vomiting +++ |
| L | 4 | F | Y | 2.5/x | 1.5 | ND | + | Nausea +++; |
| | | | | | | | | Vomiting +++; |
| | | | | | | | | Bloating + |
| Z | 5 | M | Y | 2.5/x | 0.7 | N | + | Bloating + |
| AA | 4 | F | Y | 2.5/x | 1.1 | N | ++ | Asmptomatic |
| BB | 5 | M | Y | 2.5/x | 1.2 | N | + | Asmptomatic |
| CC | 5 | F | Y | 2.5/x | 0.8 | N | NR | Night |
| | | | | | | | | terror/restless |
| | | | | | | | | sleep +++; rash +; |
| | | | | | | | | pain ++ |
| DD | 5 | F | Y | 2.5/2.5 | 0.9 | N | + | Lethargy +; |
| | | | | | | | | grumpy +; |
| | | | | | | | | bloated +; |
| | | | | | | | | headcold + |
| EE | 4 | M | Y | 2.5/x | 0.4 | ? | NR | Pain ++; grumpy |
| | | | | | | | | +; lethargy +; |
| | | | | | | | | paleness + |
| FF | 5 | F | Y | 2.5/x | 0.7 | N | NR | Pain +; |
| | | | | | | | | constipation |
| GG | 5 | F | Y | 2.5/x | 0.4 | Y | + | Asymptomatic |
| HH | 5 | F | Y | 2.5/x | 4.4 | ? | + | Vomiting +++; |
| | | | | | | | | Nausea +; |
| | | | | | | | | flatulence +; lack |
| | | | | | | | | of appetite |
| I | 8 | F | Y | 2.5/x | 4.0 | N | + | Asmptomatic |
| J | 6 | F | Y | 2.5/2.5 | 4.0 | N | +++ | Asmptomatic |
| K | 10 | M | N | 2.5/x | 4.7 | N | +++ | Vomiting +++; |
| | | | | | | | | Lethargy +++ |
| V | 10 | F | Y | 2.5/x | 0.6 | Y | ++ | Lethargy + |
| W | 10 | M | Y | 2.5/x | 3.3 | N | + | Asmptomatic |
| X | 7 | F | Y | 2.5/x | 3.3 | Y | NR | Pain ++ |
| Y | 6 | M | N | 2.5/x | 1.3 | Y | + | Pain + |
| II | 9 | F | Y | 2.5/x | 2.8 | N | + | Asmptomatic |
| JJ | 9 | F | Y | 2.5/2.5 | 7.6 | Y | ++ | Headache +; Pain |
| | | | | | | | | +; Constipation + |
| KK | 9 | F | Y | 2.5/x | 2.0 | Y | NR | Asmptomatic |
| LL | 6 | F | Y | 2.5/x | 0.6 | Y | ++ | Irritable + |
| MM | 10 | F | Y | 2.5/x | 5.6 | Y | + | Pain ++; nausea + |
| A | 14 | M | Y | 2.5/x | 4.7 | ND | + | Asmptomatic |
| B | 11 | M | Y | 2.5/2.5 | 8.5 | N | NR | Pain ++ |
| C | 17 | M | Y | 2.5/2.5 | 2.6 | N | + Protein | Pain ++; Nausea |
| | | | | | | | only | ++; Lethargy + |
| D | 17 | M | Y | 2.5/2.5 | 2.4 | N | + | Flatulence |
| F | 17 | M | Y | 2.5/x | 1.6 | N | +++ | Lethargy ++; Pain + |
| E | 11 | F | Y | 2.5/2.5 | 1.6 | N | +++ | Nausea +; Pain +; |
| | | | | | | | | Lethargy ++ |
| G | 16 | M | Y | 2.5/x | 6.8 | N | + | Asmptomatic |
| H | 11 | M | Y | 2.5/x | 1.0 | N | + | Pain + |
| P | 16 | F | Y | 2.5/x | 1.7 | Y | ++ | Nausea +++; |
| | | | | | | | | Vomiting +; |
| | | | | | | | | Lethargy ++; |
| | | | | | | | | Flatulence |
| Q | 17 | M | N | 2.5/x | 7.2 | N | +++ | Vomiting +++ |
| R | 14 | F | Y | 2.5/x | 3.5 | N | NR | Nausea +++; |
| | | | | | | | | Bloating +; Pain |
| | | | | | | | | ++; Constipation + |
| S | 13 | F | Y | 2.5/x | 3.0 | N | NR | Constipation + |
| U | 15 | M | Y | 2.5/2.5 | 4.6 | N | +++ | Pain +; Lethargy |
| | | | | | | | | +; Mouth ulcers |
| T | 17 | M | Y | 2.5/2.5 | 4.6 | Y | +++ | Asmptomatic |
| NN | 13 | F | Y | 2.5/2.5 | 9.9 | N | + | Pain + |
| PP | 13 | F | Y | 2.5/x | 3.7 | N | NR | Nausea +; |
| | | | | | | | | Diarrhoea +++; |
| | | | | | | | | Lethargy ++ |

x denotes an allele other than HLA-DQ2.5 or HLA-DQ8

Short-term oral wheat challenge was performed as previously described for adults with CD [ref. 11], however the amount of bread consumed daily was modified for the younger age groups: 3-5 yr 1 slice of bread, 6-10 yr two slices, and 11-18 yr three slices. This corresponded to a similar amount of daily gluten intake across all ages groups when median weight (based on weight-for-age percentile charts from the cdc.gov website) was considered (approximately 0.19-0.23 g/kg gluten).

Blood for T-cell studies was collected by trained paediatric phlebotomists in Lithium heparin vacutainers before (D0) and six days (D6) after commencing the oral challenges. Venesection volume was determined by weight following WHO recommendations [Howie, 2011]. Patients filled in symptom diaries where symptom type and severity (mild, moderate, or severe) were described for the six days following gluten challenge.

### Antigens

To optimize assessment of peptides with the limited blood from paediatric donors, a modified library containing both wild-type and in silico deamidated versions of the most immunogenic wheat peptide sequences described previously [ref. 11] was tested (Table 7; n=70, 37 wild-type and 33 in silico deamidated). When blood volume enabled, a series of peptides known to be immunostimulatory in vivo in a large adult CD cohort were additionally assessed: barley hordein (n=22, all deamidated), rye secalin (n=30, all deamidated), and oats avenin (n=2, 1 deamidated) [refs. 11,19].

TABLE 7
Peptide information

| Wheat derived peptides | Barley derived peptides | Rye derived peptides |
|---|---|---|
| FPQPEQEFPQPQQ (SEQ | EQPFPEQPFPEQP (SEQ | ELPLQPEQPFPQP (SEQ |
| ID NO: 31) | ID NO: 32) | ID NO: 33) |
| FPQPQQQFPQPQQ (SEQ | FPEQPIPEQPQPYP (SEQ | FPQSEQPEQPFPQP (SEQ |
| ID NO: 34) | ID NO: 35) | ID NO: 36) |
| GLERPWQEQPLPPQ (SEQ | FPEQPVPEQPQPYP | FPQTEQPEQPFPQP (SEQ |
| ID NO: 37) | (SEQ ID NO: 38) | ID NO: 39) |
| GLERPWQQQPLPPQ | FSFSEQPEQPFPLQ (SEQ | IIPEQPEQPFPLQ (SEQ ID |
| (SEQ ID NO: 40) | ID NO: 41) | NO: 42) |
| GQPGYYPTSPQQIGQ | NPLQPEQPFPLQPQPP | IISEQPEQPFPLQ (SEQ ID |
| (SEQ ID NO: 43) | (SEQ ID NO: 44) | NO: 45) |
| GQQGYYPISPQQSGQ | PEQPFPEQPQPYPQQP | LPFPQPEQPFVVV (SEQ |
| (SEQ ID NO: 46) | (SEQ ID NO: 47) | ID NO: 48) |
| GQSGYYPTSPQQS (SEQ | PEQPFQPEQPFPQQ | PAPIQPEQPFPQQ (SEQ |
| ID NO: 49) | (SEQ ID NO: 50) | ID NO: 51) |
| IQVDPSGEVEWPQQ (SEQ | PEQPQPFPEQPVPQQP | PEQIIPEQPEQPS (SEQ ID |
| ID NO: 52) | (SEQ ID NO: 53) | NO: 54) |
| IQVDPSGEVQWPQQ (SEQ | PFPLQPEQPFPWQ (SEQ | PEQPFPEQPEQII (SEQ ID |
| ID NO: 55) | ID NO: 56) | NO: 57) |
| IQVDPSGQVEWPQQ (SEQ | PFPWQPEQPFPQP (SEQ | PEQPFPEQPQQII (SEQ ID |
| ID NO: 58) | ID NO: 59) | NO: 60) |
| IQVDPSGQVQWPQQ | PQPFPEQPIPEQPQPY | PEQPYPEQPFPQQ (SEQ |
| (SEQ ID NO: 61) | (SEQ ID NO: 62) | ID NO: 63) |
| LPYPQPELPYPQP (SEQ | PQPYPEQPQPFPQQPP | PFLLQPEQPFSQP (SEQ |
| ID NO: 64) | (SEQ ID NO: 65) | ID NO: 66) |
| LPYPQPQLPYPQP (SEQ | QEFPQPEQPFPQQ (SEQ | PFPEQPEQIIPQQ (SEQ |
| ID NO: 67) | ID NO: 68) | ID NO: 69) |
| LQPFPQPELPFPQP (SEQ | QPFPEQPFPEQPQPY | PFPEQPEQIISQQ (SEQ ID |
| ID NO: 70) | (SEQ ID NO: 71) | NO: 72) |
| LQPFPQPELPYLQP (SEQ | QPFPQPEQPFPLQ (SEQ | PFPEQPEQPFPQQ (SEQ |
| ID NO: 73) | ID NO: 74) | ID NO: 75) |
| LQPFPQPELPYPQP (SEQ | QPFPQPEQPFRQQ (SEQ | PFPERPEQPFPQP (SEQ |
| ID NO: 76) | ID NO: 77) | ID NO: 78) |
| LQPFPQPELPYSQP (SEQ | QPFPQPEQPFSWQ (SEQ | PFPLQPEQPFSQP (SEQ |
| ID NO: 79) | ID NO: 80) | ID NO: 81) |
| LQPFPQPQLPYLQP (SEQ | QPFPQPEQPIPYQ (SEQ | PFPLQPEQPVPEQPQ |
| ID NO: 82) | ID NO: 83) | (SEQ ID NO: 84) |
| LQPFPQPQLPYPQP (SEQ | QPQPFPEQPIPLQ (SEQ | PTPIQPEQPFPQR (SEQ |
| ID NO: 85) | ID NO: 86) | ID NO: 87) |
| LQPFPQPQLPYSQP (SEQ | QPQPFPEQPIPQQ (SEQ | QLFPLPEQPFPQP (SEQ |
| ID NO: 88) | ID NO: 89) | ID NO: 90) |
| LQQPFPQPQLPFPQP (SEQ | QPQPYPEQPQPYP (SEQ | QPEQPFPLQPEQPVP |
| ID NO: 91) | ID NO: 92) | (SEQ ID NO: 93) |
| LQQQCSPVAMPQRLAR | SYPVQPEQPFPQP (SEQ | QPFPQPEQELPLQ (SEQ |
| (SEQ ID NO: 94) | ID NO: 95) | ID NO: 96) |
| PEQPFPEQPEQPF (SEQ ID | | QPFPQPEQPFPQS (SEQ |
| NO: 97) | | ID NO: 98) |
| PEQTFPEQPQLPF (SEQ | Avenin derived peptides | QPFPQPEQPIPQQ (SEQ |
| ID NO: 99) | | ID NO: 100) |
| PFPEQPEQEFPQP (SEQ ID | YQPYPEQEQPILQQ | QPFPQPEQPTPIQ (SEQ |
| NO: 101) | (SEQ ID NO: 102) | ID NO: 103) |
| PFPEQPEQPFPQP (SEQ ID | YQPYPEQQQPILQQ | QPFPQPEQQLPLQ (SEQ |
| NO: 104) | (SEQ ID NO: 105) | ID NO: 106) |
| PFPEQPEQPYPQQ (SEQ | | QSIPQPEQPFPQP (SEQ |
| ID NO: 107) | | ID NO: 108) |
| PFPLQPEQPFPQP (SEQ ID | | VGPSGEVEWPQQ (SEQ |
| NO: 109) | | ID NO: 110) |
| PFPLQPEQPFPQQ (SEQ ID | | YPEQPFPEQPEQPY (SEQ |
| NO: 111) | | ID NO: 112) |
| PFPLQPQQPFPQP (SEQ ID | (pE)QQPQQSFPEQERPF | YSPYQPEQPFPQP (SEQ |
| NO: 113) | (SEQ ID NO: 114) | ID NO: 115) |
| PFPLQPQQPFPQQ (SEQ ID | (pE)XPQQQQXPEQPQQ | |
| NO: 116) | F (SEQ ID NO: 117) | |
| PFPQQPQQPFPQP (SEQ ID | (pE)QQSEESEQPFQPQP | |
| NO: 118) | (SEQ ID NO: 119) | |
| PFPQQPQQPYPQQ (SEQ | (pE)QPPFSEEQEQPLPQ | |
| ID NO: 120) | (SEQ ID NO: 121) | |
| PFPQQPQQQFPQP (SEQ | (pE)QPPFSEQQESPSFSQ | |
| ID NO: 122) | (SEQ ID NO: 123) | |
| PFPWQPEQPFPQQ (SEQ | (pE)GIIPEQPAQLEGI | |
| ID NO: 124) | (SEQ ID NO: 125) | |
| PFPWQPQQPFPQQ (SEQ | (pE)QPFRPEQPYPQPQP | |
| ID NO: 126) | (SEQ ID NO: 127) | |
| PIPEQPEQPFPLQ (SEQ ID | QPQQPQQSFPQQQRPF | |
| NO: 128) | (SEQ ID NO: 129) | |
| PIPQQPQQPFPLQ (SEQ ID | QQXSQPQXPQQQQXPQ | |
| NO: 130) | QPQQF (SEQ ID NO: 131) | |
| PQPFLPELPYPQP (SEQ ID | QPQPFPQQSEQSQQPFQ | |
| NO: 132) | PQPF (SEQ ID NO: 133) | |
| PQPFLPQLPYPQP (SEQ ID | QQPPFSQQQQQPLPQ | |
| NO: 134) | (SEQ ID NO: 135) | |
| PQQPFPQQPQQPF (SEQ | QQQQPPFQQQQSPFSQ | |
| ID NO: 136) | QQQ (SEQ ID NO: 137) | |
| PQQTFPQQPQLPF (SEQ | VQGQGIIQPQQPAQL | |
| ID NO: 138) | (SEQ ID NO: 139) | |
| PTPIQPEQPFPQQ (SEQ ID | PFRPQQPYPQPQPQ | |
| NO: 140) | (SEQ ID NO: 141) | |
| PTPIQPQQPFPQQ (SEQ ID | | |
| NO: 142) | | |
| QAFPQPEQTFPHQ (SEQ | | |
| ID NO: 143) | | |
| QAFPQPQQTFPHQ (SEQ | | |
| ID NO: 144) | | |
| QFIQPEQPFPQQ (SEQ ID | | |
| NO: 145) | | |
| QFIQPQQPFPQQ (SEQ ID | | |
| NO: 146) | | |
| QPFPQLEQPEQPF (SEQ ID | | |
| NO: 147) | | |
| QPFPQLQQPQQPF (SEQ | | |
| ID NO: 148) | | |
| QPFPQPEQPFCQQ (SEQ | | |
| ID NO: 149) | | |
| QPFPQPEQPFPWQ (SEQ | | |
| ID NO: 150) | | |
| QPFPQPEQPFSQQ (SEQ ID | | |
| NO: 151) | | |
| QPFPQPEQPIPVQ (SEQ ID | | |
| NO: 152) | | |
| QPFPQPEQPQLPF (SEQ ID | | |
| NO: 153) | | |
| QPFPQPEQTFPQQ (SEQ | | |
| ID NO: 154) | | |
| QPFPQPQQPFCQQ (SEQ | | |
| ID NO: 155) | | |
| QPFPQPQQPFPWQ (SEQ | | |
| ID NO: 156) | | |
| QPFPQPQQPFSQQ (SEQ | | |
| ID NO: 157) | | |
| QPFPQPQQPIPVQ (SEQ ID | | |
| NO: 158) | | |
| QPFPQPQQPQLPF (SEQ ID | | |
| NO: 159) | | |
| QPFPQPQQTFPQQ (SEQ | | |
| ID NO: 160) | | |
| QPFTQPEQPTPIQ (SEQ ID | | |
| NO: 161) | | |
| QPFTQPQQPTPIQ (SEQ ID | | |
| NO: 162) | | |
| QQFSQPEQQFPQP (SEQ | | |
| ID NO: 163) | | |
| QQFSQPQQQFPQP (SEQ | | |
| ID NO: 164) | | |
| TIPEQPEQPFPLQ (SEQ ID | | |
| NO: 165) | | |
| TIPQQPQQPFPLQ (SEQ ID | | |
| NO: 166) | | |
| VAHAIIMHQQQQQQQE | | |
| (SEQ ID NO: 167) | | |
| YEVIRSLVLRTLPN (SEQ | | |
| ID NO: 168) | | |

The seven sequences implicated in paediatric CD based on intestinal T cell line reactivity [ref. 15] were also assessed, in both wild-type and deamidated versions at a 50% mixture of Leucine and Isoleucine (denoted by x; Table 7).

The screening library was custom synthesized and the identity of each peptide was confirmed by LC-MS (GL Biochem, Minhang, China). Additional high quality (>80%) peptides were synthesised by Pepscan (Lelystad, Netherlands), GL Biochem, or Purar Chemicals (Melbourne, Victoria, Australia). Comprehensive gliadin (n=1535), hordein (n=1444), and secalin (n=350) peptide libraries consisting of wildtype and in silico deamidated sequences [ref. 11] were used to screen TCC to establish redundancy of peptide recognition.

### IFN-γ ELISpot

PBMC were isolated from whole blood using Ficoll-Paque™ Plus density-gradient centrifugation (GE Healthcare). IFN-γ ELISpot (Mabtech) assays were performed and analyzed as previously described [ref. 11]. In brief, PBMC were incubated overnight with individual peptides, with medium alone as negative control, and with one or more positive controls including Tetanus toxoid (TT; CSL, Australia), phytohemagglutinin-L (PHA-L; Sigma USA), or CEF cocktail (Mabtech). Spot forming units (SFU) in individual wells were counted using an automated ELISPOT reader (AID ELISPOT Reader System, AID Autoimmun Diagnostika GmbH; Strassberg, Germany). Wells showing more than 10 SFU and >3× the SFU counted in wells with PBMC incubated with medium alone were regarded as “positive”. Dominance scores for each peptide were defined using the IFN-gamma response elicited as a proportion of the most active peptide screened, and then averaged across each participant group. Responses were normalised to one million PBMC input for comparisons. EC50 values were calculated using Prism 6.0 software on a dose curve containing 8 peptide concentrations ranging from 0.1-50 ug/ml, and equals the peptide half maximal peptide concentration.

### T Cell Cloning

TCC were generated as previously described [ref. 11]. Briefly, CFSE-labeled PBMC were incubated with antigen for 7 days in IMDM complete media supplemented with 5% heat-inactivated pooled human serum (PHS), 2 mM GlutaMAX™, 100 μM MEM non-essential amino acids (both from Gibco, Invitrogen), and 50 μM 2-mercaptoethanol (Sigma). Proliferating cells were sorted with one cell per well in 96-well plates and incubated in the presence of IL-2, IL-4, anti-CD3 mAb, irradiated allogeneic PBMC and JY-EBV (an Epstein-Barr virus-immortalised B cell lymphoblastoid line). TCC were expanded and maintained in IL-2 and IL-4 and tested for specificity by ELISpot as described above with minor modifications. TCC (1000-2000/well) were incubated with relevant peptide and irradiated HLA-matched PBMC or HLA-DQ2.5-expressing T2 cells as antigen-presenting cells (25,000-50,000/well). Epitopes recognized by TCC were tested for HLA-DQ2.5 restriction using an anti-human blocking antibody specific for HLA-DQ and the HLA-DQ2.5-expressing T2 cells, TCR Vbeta usage by the IOTest Beta Mark TCR V kit (Beckman Coulter), and with lysine scans to work out minimal epitopes 20.

### IFN-γ Secretion Assay

Either fresh or cryopreserved PBMC from known T cell responders were rested overnight in IMDM complete media. CD4+ T cells were enriched using the EasySep Negative Selection Human CD4+ T cell enrichment kit (Stem Cell Technologies), following manufacturer's recommendations. CD4+ T cells were stimulated with or without 50 ug/ml peptide, in addition to 10 ug/ml purified anti-CD28 antibody and 1.25 ug/ml anti-CD49d antibody (both from Biolegend), and autologous PBMC at a 1:1 ratio, in 96-well round bottom plates in replicate wells containing a final volume of 150 ul IMDM complete media containing 20 ug/ml DNase I (Roche). The MACS IFN-γ secretion assay—Detection kit (FITC) human (Miltenyi Biotec) was used to enable cell sorting of IFN-γ secreting, antigen-specific CD4+ T cells, following manufacturer's recommendations. In addition to IFN-γ-FITC, cells were co-stained with CD4-APC and CD14-PerCP (BD Biosciences), CD69-PECy7 (Biolegend), and propidium iodide (Sigma). Ag-specific (IFN-γ+CD69+CD4+) cells were single-cell sorted into 96-well PCR plates (eppendorf) up to 80 wells and one column left as non-template controls, on a BD FACS Aria. Wells were capped with strip lids, and stored frozen for later processing.

### Statistical Analysis

Two-tailed Wilcoxon signed rank tests or Kruskal-Wallis tests were performed for comparisons between two or more groups of paired data respectively. Contingency analysis was performed using either the Fisher's exact test or Chi-Square test. Statistical tests were performed on GraphPad Prism version 6 software. P values ≦0.05 were considered significant.

### Results

Gluten-Specific T Cells are Induced by Wheat Challenge in Children with CD and Gluten Peptide Specificity and Dominance is Comparable to Adults

Positive IFN-γ ELISpot responses following oral wheat challenge were detected in most CD participants to gluten and gliadin proteins (26/40; 65%) and gluten peptides (29/40; 72.5%) (Table 8).

TABLE 8
Wheat-derived T cell epitope hierarchy in pediatric coeliac disease

| | 3-5yrs | | | | | | | | | | |
|---|---|---|---|---|---|---|---|---|---|---|---|
| Patient ID | M | N | L | Z | AA | BB | HH | GG | FF | DD | |
| Age | 4 H | 3 | 4 | 5 | 4 | 5 | 5 | 5 | 5 H | 5 H | |
| >10SFU | 10~ | 10~ | 10~ | 10~ | 10~ | 10~ | 10~ | 10~ | 10~ | 10~ | |
| (cut-off 1) | | | | | | | | | | | |
| No Antigen | 0 | 0 | 0 | 0.5 | 0 | 2 | 1 | 2.5 | 0 | 1 | |
| 3x No | 0 | 0 | 0 | 1.5 | 0 | 6 | 3 | 7.5 | 0 | 3 | |
| Antigen | | | | | | | | | | | |
| (cut-off 2) | | | | | | | | | | | |
| D-gli 100 | 51.5*** | 8** | 21.5*** | 1.5** | 1 | 9 | 78.5*** | 13.5 | 4** | 7.5 | |
| Gli 100 | 44.5 | 3** | 17.5*** | 1.5** | 1.5 | 32 | 87*** | 12.5 | 3** | 5.5 | |
| D-Glu 100 | 77.5*** | 4** | 20*** | 3.5** | 8 | 43 | 75.5*** | 11.5 | 3** | 8 | |
| Glu 100 | 47.5 | 4.5** | 10.5 | 1** | 2 | 30 | 86.5*** | 21.5 | 4.5** | 13 | |
| Max peptide | 48 | 19 | 11 | 13 | 60 | 44 | 13 | 14.5 | 12 | 16.5 | |
| QPFPQPEQPFPWQ | 48+ | 19+ | 1 | | 60+ | 18′′′ | | 8 | 9 | | |
| (SEQ ID NO: 150) | | | | | | | | | | | |
| LQPRPQPELPYPQP | 45+ | 7 | 11+ | | 36′′′ | 24′′′ | | 6 | 8 | | |
| (SEQ ID NO: 176) | | | | | | | | | | | |
| LQPFPQPELPFPQP | 41+ | 4 | 4 | 24′′ | 26′′′ | | 12+ | 10+ | | | |
| (SEQ ID NO: 70) | | | | | | | | | | | |
| LQPFPQPELPYSQP | 36+ | 3 | 2 | | 13′ | 22′′′ | | 11+ | 7 | | |
| (SEQ ID NO: 79) | | | | | | | | | | | |
| LPYPQPELPYPQP | 33′′′ | 6 | 11+ | | 33′′′ | 44+ | | 9 | 5 | | |
| (SEQ ID NO: 64) | | | | | | | | | | | |
| QPFPQPEQPFPWQP | 27.5′′′ | 7 | 3.5 | 13+ | 52.5+ | 28.5′′′ | 10+ | 14.5+ | 5.5 | 9 | |
| (SEQ ID NO: 8) | | | | | | | | | | | |
| LQPFPQPELPYPQP | 31′′′ | 5.5 | 8 | 10+ | 29′′′ | 40.5 | 13+ | 14+ | 12+ | 16.5+ | |
| Q | | | | | | | | | | | |
| (SEQ ID NO: 7) | | | | | | | | | | | |
| QPFPQPEQPIPVQ | 29′′′ | 2 | 2 | | 53+ | 10′′ | | 11+ | 4 | | |
| (SEQ ID NO: 152) | | | | | | | | | | | |
| PFPQQPQQPFPQP | 4 | 2 | 2 | | 3 | 0 | | 12+ | 1 | | |
| (SEQ ID NO: 118) | | | | | | | | | | | |
| PEQPFPEQPEQPF | 4 | 0 | 0 | | 9 | 4 | | 11+ | 0 | | |
| (SEQ ID NO: 97) | | | | | | | | | | | |
| PEQTFPEQPQLPF | 2 | 1 | 3 | | 1 | 0 | | 4 | 1 | | |
| (SEQ ID NO: 99) | | | | | | | | | | | |
| FPQPEQEFPQPQQ | 13′′ | 3 | 0 | | 13′ | 10′′ | | 5 | 5 | | |
| (SEQ ID NO: 31) | | | | | | | | | | | |
| YEVIRSLVLRTLPN | 0 | 2 | 0 | | 1 | 1 | | 3 | 9 | | |
| (SEQ ID NO: 168) | | | | | | | | | | | |
| PFPLQPEQPFPQP | 24′′′ | 7 | 1 | | 24′′ | 22′′′ | | 8 | 10+ | | |
| (SEQ ID NO: 109) | | | | | | | | | | | |
| LPYPQPQLPYPQP | 13′′ | 3 | 1 | | 14′′ | 22′′′ | | 7 | 5 | | |
| (SEQ ID NO: 67) | | | | | | | | | | | |
| LQPFPQPQLPYPQP | 22′′′ | 4 | 3 | | 21′′ | 17′′ | | 5 | 1 | | |
| (SEQ ID NO: 85) | | | | | | | | | | | |
| QPFPQPQQPFSQQ | 13′′ | 3 | 1 | | 9 | 12′′ | | 3 | 2 | | |
| (SEQ ID NO: 157) | | | | | | | | | | | |
| FPQPQQQFPQPQQ | 6 | 1 | 3 | | 0 | 5 | | 4 | 2 | | |
| (SEQ ID NO: 34) | | | | | | | | | | | |
| PFPEQPEQPYPQQ | 11′ | 1 | 1 | | 6 | 4 | | 5 | 3 | | |
| (SEQ ID NO: 107) | | | | | | | | | | | |
| PIPQQPQQPFPLQ | 0 | 1 | 1 | | 2 | 1 | | 7 | 7 | | |
| (SEQ ID NO: 130) | | | | | | | | | | | |
| QFIQPEQPFPQQ | 8 | 6 | 2 | | 7 | 6 | | 6 | 1 | | |
| (SEQ ID NO: 145) | | | | | | | | | | | |
| GQSGYYPTSPQQS | 2 | 0 | 4 | | 2 | 3 | | 5 | 0 | | |
| (SEQ ID NO: 49) | | | | | | | | | | | |
| QPFPQPQQPIPVQ | 9 | 5 | 0 | | 17′′ | 9 | | 9 | 9 | | |
| (SEQ ID NO: 158) | | | | | | | | | | | |
| TIPEQPEQPFPLQ | 2 | 3 | 1 | | 11′ | 3 | | 5 | 3 | | |
| (SEQ ID NO: 165) | | | | | | | | | | | |
| PFPEQPEQPFPQP | 19′′ | 2 | 3 | | 24′′ | 6 | | 5 | 2 | | |
| (SEQ ID NO: 104) | | | | | | | | | | | |
| GIIQPQQPAQL | | | | | 0 | 0 | | 5 | 3 | | |
| (SEQ ID NO: 169) | | | | | | | | | | | |
| FLQPEQPFPEQPEQ | | | | | 12′ | 10′′ | | 3 | 3 | | |
| PYPEQPEQPFPQ | | | | | | | | | | | |
| (SEQ ID NO: 170) | | | | | | | | | | | |
| PQQPQQSFPQQQQP | | | | | 0 | 0 | | 7 | 2 | | |
| A | | | | | | | | | | | |
| (SEQ ID NO: 171) | | | | | | | | | | | |
| GLERPWQEQPLPPQ | 0 | 0 | 2 | | 1 | 6 | | 5 | 6 | | |
| (SEQ ID NO: 37) | | | | | | | | | | | |
| | 6-10 yrs | | | | | | | | | | |
| Patient ID | J | K | V | W | Y | II | JJ | LL | MM | | |
| Age | 6 H | 10 | 10 | 10 | 6 | 9 | 9H | 6 | 10 | | |
| >10SFU | 10 | 10~ | 10~ | 10 | 10~ | 10~ | 10~ | 10~ | 10 | | |
| (cut-off 1) | | | | | | | | | | | |
| No Antigen | 3.5 | 1 | 3 | 9.5 | 2 | 2.5 | 0 | 0.5 | 3.5 | | |
| 3x No | 10.5~ | 3 | 9 | 28.5~ | 6 | 7.5 | 0 | 1.5 | 10.5~ | | |
| Antigen | | | | | | | | | | | |
| (cut-off 2) | | | | | | | | | | | |
| D-gli 100 | 70.5 | 42 | 5 | 6** | 26 | 15.5*** | 3 | 1 | 15 | | |
| Gli 100 | 21 | 13 | 4.5 | 11** | 15.5 | 34*** | 20 | 8 | 24.5 | | |
| D-Glu 100 | 70 | 17.5 | 13.5* | 16.5*** | 17.5* | 27*** | 25 | 18.5 | 15.5 | | |
| Glu 100 | 12 | 8 | 8 | 16.5** | 16.5*** | 24.5*** | 5.5 | 5.5 | 23.5 | | |
| Max pepide | 196 | 53 | 35 | 30 | 16 | 15 | 48 | 57 | 27 | | |
| QPFPQPEQPFPWQ | 13′′′ | 26′′ | 34+ | 1 | 8 | 4 | 47+ | 6 | 4 | | |
| (SEQ ID NO: 150) | | | | | | | | | | | |
| LQPRPQPELPYPQP | 146+ | 34′′′ | 30+ | 14 | 11′′′ | 10′′′ | 44+ | 38′′′ | 6 | | |
| (SEQ ID NO: 176) | | | | | | | | | | | |
| LQPFPQPELPFPQP | 112′′′ | 16′′ | 22′′′ | 15 | 6 | 11+ | 15′′ | 43+ | 15′′′ | | |
| (SEQ ID NO: 70) | | | | | | | | | | | |
| LQPFPQPELPYSQP | 71′′ | 9 | 19′′′ | 25 | 12+ | 15+ | 20′′′ | 39′′′ | 6 | | |
| (SEQ ID NO: 79) | | | | | | | | | | | |
| LPYPQPELPYPQP | 196+ | 53+ | 35+ | 15 | 16+ | 11+ | 33′′′ | 45+ | 3 | | |
| (SEQ ID NO: 64) | | | | | | | | | | | |
| QPFPQPEQPFPWQP | 95′′′ | 18.5′′ | 25+ | 9 | 10′′′ | 11+ | 40.5+ | 20.5′′ | 11′′′ | | |
| (SEQ ID NO: 8) | | | | | | | | | | | |
| LQPFPQPELPYPQP | 99.5′′′ | 24′′ | 30+ | 6.5 | 10.5′′′ | 8.5 | 42+ | 57+ | 8 | | |
| Q | | | | | | | | | | | |
| (SEQ ID NO: 7) | | | | | | | | | | | |
| QPFPQPEQPIPVQ | 126′′′ | 12′ | 27+ | 1 | 9 | 5 | 46+ | 9 | 9 | | |
| (SEQ ID NO: 152) | | | | | | | | | | | |
| PFPQQPQQPFPQP | 5 | 3 | 3 | 6 | 6 | 6 | 1 | 3 | 1 | | |
| (SEQ ID NO: 118) | | | | | | | | | | | |
| PEQPFPEQPEQPF | 24′ | 22′′ | 3 | 24 | 2 | 2 | 4 | 12′′ | 5 | | |
| (SEQ ID NO: 97) | | | | | | | | | | | |
| PEQTFPEQPQLPF | 179+ | 47′′′ | 4 | 12 | 1 | 4 | 1 | 0 | 4 | | |
| (SEQ ID NO: 99) | | | | | | | | | | | |
| FPQPEQEFPQPQQ | 158+ | 35′′′ | 9 | 21 | 3 | 13+ | 15′′ | 1 | 11′′′ | | |
| (SEQ ID NO: 31) | | | | | | | | | | | |
| YEVIRSLVLRTLPN | 4 | 2 | 3 | 30+ | 1 | 2 | 0 | 1 | 10 | | |
| (SEQ ID NO: 168) | | | | | | | | | | | |
| PFPLQPEQPFPQP | 52′′ | 37′′′ | 21′′′ | 29+ | 9 | 13+ | 34+ | 8 | 27+ | | |
| (SEQ ID NO: 109) | | | | | | | | | | | |
| LPYPQPQLPYPQP | 75′′ | 24′′ | 18′′′ | 21 | 16+ | 11+ | 18′′ | 25′′′ | 8 | | |
| (SEQ ID NO: 67) | | | | | | | | | | | |
| LQPFPQPQLPYPQP | 93′′′ | 10′ | 10′′ | 10 | 14+ | 12+ | 333′′′ | 29′′′ | 5 | | |
| (SEQ ID NO: 85) | | | | | | | | | | | |
| QPFPQPQQPFSQQ | 13\hat{ } | 21′′ | 5 | 12 | 6 | 12+ | 18′′ | 1 | 6 | | |
| (SEQ ID NO: 157) | | | | | | | | | | | |
| FPQPQQQFPQPQQ | 1 | 6 | 2 | 19 | 6 | 11+ | 2 | 1 | 2 | | |
| (SEQ ID NO: 34) | | | | | | | | | | | |
| PFPEQPEQPYPQQ | 9 | 5 | 7 | 14 | 5 | 11+ | 13′′ | 3 | 3 | | |
| (SEQ ID NO: 107) | | | | | | | | | | | |
| PIPQQPQQPFPLQ | 1 | 3 | 9 | 12 | 5 | 11+ | 3 | 4 | 14′′′ | | |
| (SEQ ID NO: 130) | | | | | | | | | | | |
| QFIQPEQPFPQQ | 30′ | 17′′ | 19′′′ | 5 | 3 | 11+ | 9 | 0 | 8 | | |
| (SEQ ID NO: 145) | | | | | | | | | | | |
| GQSGYYPTSPQQS | 6 | 1 | 3 | 16 | 0 | 11+ | 1 | 4 | 11′′′ | | |
| (SEQ ID NO: 49) | | | | | | | | | | | |
| QPFPQPQQPIPVQ | 136′′′ | 26′′ | 11′′ | 8 | 3 | 7 | 48+ | 4 | 13′′′ | | |
| (SEQ ID NO: 158) | | | | | | | | | | | |
| TIPEQPEQPFPLQ | 18\hat{ } | 6 | 0 | 7 | 2 | 5 | 6 | 3 | 1 | | |
| (SEQ ID NO: 165) | | | | | | | | | | | |
| PFPEQPEQPFPQP | 51′′ | 25′′ | 16′′′ | 13 | 4 | 8 | 18′′ | 4 | 9 | | |
| (SEQ ID NO: 104) | | | | | | | | | | | |
| GIIQPQQPAQL | 3 | 22′′ | 5 | 19 | 2 | 11+ | | 1 | 3 | | |
| (SEQ ID NO: 169) | | | | | | | | | | | |
| FLQPEQPFPEQPEQ | 22′ | 20′′ | 12′′ | 8 | 6 | 1 | | 15′′ | 20+ | | |
| PYPEQPEQPFPQ | | | | | | | | | | | |
| (SEQ ID NO: 170) | | | | | | | | | | | |
| PQQPQQSFPQQQQP | 1 | 23′′ | 10′′ | 12 | 3 | 8 | | 0 | 10 | | |
| A | | | | | | | | | | | |
| (SEQ ID NO: 171) | | | | | | | | | | | |
| GLERPWQEQPLPPQ | 3 | 0 | 7 | 23 | 9 | 3 | 0 | 1 | 11′′′ | | |
| (SEQ ID NO: 37) | | | | | | | | | | | |
| | 11-17 yrs | | | | | | | | | | |
| Patient ID | A | D | F | E | G | H | P | Q | U | NN | |
| Age | 14 | 17 H | 17 | 11 H | 16 | 11 | 16 | 17 | 15 H | 13 H | 17 H |
| >10SFU | 10~ | 10~ | 10~ | 10~ | 10~ | 10~ | 10~ | 10~ | 10~ | 10~ | 10~ |
| (cut-off 1) | | | | | | | | | | | |
| No Antigen | 0.5 | 1 | 1 | 0.5 | 0 | 0.5 | 7.2 | 0 | 2.5 | 0.5 | 0.5 |
| 3x No | 1.5 | 3 | 3 | 1.5 | 0 | 1.5 | 21.6~ | 0 | 7.5 | 1.5 | 1.5 |
| Antigen | | | | | | | | | | | |
| (cut-off 2) | | | | | | | | | | | |
| D-gli 100 | 10 | 44.5*** | 138 | 78.5 | 7.5** | 27.5*** | 14 | 13 | 6.5 | 2.5** | 8 |
| Gli 100 | 6 | 38.5*** | 57 | 12.5 | 8.5** | 16 | 28 | 50 | 17 | 4** | 23 |
| D-Glu 100 | 8 | 37*** | 127 | 51 | 3** | 12.5 | 37.5 | 56.5 | 26.5 | 4.5** | 48 |
| Glu 100 | 5 | 25.5 | 60 | 8.5 | 5.2** | 6 | 21 | 40 | 9.5 | 3** | 12.5 |
| Max peptide | 20 | 30.5 | 178 | 224 | 15 | 17 | 64 | 97 | 81 | 10 | 95 |
| QPFPQPEQPFPWQ | 5 | 17′′′ | 77′′′ | 61′′ | 5 | 6 | 43′′′ | 54′′′ | 34′′′ | 2 | 83+ |
| (SEQ ID NO: 150) | | | | | | | | | | | |
| LQPRPQPELPYPQP | 6 | 18′′′ | 178+ | 142′′′ | 4 | 10′′′ | 64+ | 97+ | 81+ | 0 | 89+ |
| (SEQ ID NO: 176) | | | | | | | | | | | |
| LQPFPQPELPFPQP | 5 | 13′′′ | 107′′′′ | 103′′′ | 7 | 16+ | 33′′′ | 65′′′ | 54′′′′ | 3 | 59′′′2 |
| (SEQ ID NO: 70) | | | | | | | | | | | |
| LQPFPQPELPYSQP | 3 | 6 | 81′′′ | 104′′′ | 5 | 15+ | 37′′′ | 29′′ | 25′′ | 2 | 53′′′ |
| (SEQ ID NO: 79) | | | | | | | | | | | |
| LPYPQPELPYPQP | 2 | 12′′ | 161+ | 111′′′ | 7 | 10′′′ | 36′′′ | 78+ | 44′′′ | 1 | 62′′′ |
| (SEQ ID NO: 64) | | | | | | | | | | | |
| QPFPQPEQPFPWQP | 3.5 | 30.5+ | 84.5′′′ | 44′ | 8 | 8.5 | 20 | 63 | 17′′ | 3.5 | 67.5+ |
| (SEQ ID NO: 8) | | | | | | | | | | | |
| LQPFPQPELPYPQP | 2.5 | 27+ | 166.5+ | 152′′′ | 10′′′ | 8 | 41.5′′′ | 67.5′′′ | 45′′′ | 2 | 66.5′′′ |
| Q | | | | | | | | | | | |
| (SEQ ID NO: 7) | | | | | | | | | | | |
| QPFPQPEQPIPVQ | 5 | 20′′′ | 103′′′ | 76′′ | 3 | 10′′′ | 42′′′ | 51′′′ | 27′′ | 1 | 95 |
| (SEQ ID NO: 152) | | | | | | | | | | | |
| PFPQQPQQPFPQP | 0 | 2 | 1 | 0 | 0 | 2 | 14 | 9 | 2 | 2 | 0 |
| (SEQ ID NO: 118) | | | | | | | | | | | |
| PEQPFPEQPEQPF | 2 | 1 | 2 | 14\hat{ } | 2 | 9 | 10 | 11′ | 5 | 10+ | 15′ |
| (SEQ ID NO: 97) | | | | | | | | | | | |
| PEQTFPEQPQLPF | 3 | 0 | 1 | 19\hat{ } | 15+ | 9 | 4 | 2 | 1 | 0 | 0 |
| (SEQ ID NO: 99) | | | | | | | | | | | |
| FPQPEQEFPQPQQ | 6 | 7 | 21′ | 49′′ | 4 | 9 | 11 | 27′′ | 15′ | 3 | 26′′ |
| (SEQ ID NO: 31) | | | | | | | | | | | |
| YEVIRSLVLRTLPN | 20+ | 0 | 2 | 1 | 1 | 3 | 49+ | 2 | 3 | 0 | 1 |
| (SEQ ID NO: 168) | | | | | | | | | | | |
| PFPLQPEQPFPQP | 6 | 15′′′ | 35′ | 30′ | 2 | 11′′′ | 27′′′ | 43′′′ | 23′′ | 5 | 28′′ |
| (SEQ ID NO: 109) | | | | | | | | | | | |
| LPYPQPQLPYPQP | 3 | 5 | 120′′′ | 61′′ | 2 | 13+ | 17 | 53′′′ | 25′′ | 1 | 49′′′ |
| (SEQ ID NO: 67) | | | | | | | | | | | |
| LQPFPQPQLPYPQP | 1 | 10′′ | 64′′ | 86′′ | 3 | 14+ | 25′′ | 38′′ | 35 | 0 | 51′′′ |
| (SEQ ID NO: 85) | | | | | | | | | | | |
| QPFPQPQQPFSQQ | 9 | 9 | 12\hat{ } | 16\hat{ } | 1 | 2 | 10 | 28′′ | 22′′ | 2 | 30′′ |
| (SEQ ID NO: 157) | | | | | | | | | | | |
| FPQPQQQFPQPQQ | 2 | 5 | 4 | 12\hat{ } | 1 | 5 | 4 | 10′ | 4 | 0 | 1 |
| (SEQ ID NO: 34) | | | | | | | | | | | |
| PFPEQPEQPYPQQ | 0 | 2 | 8 | 14\hat{ } | 1 | 1 | 15 | 19′ | 2 | 1 | 14′ |
| (SEQ ID NO: 107) | | | | | | | | | | | |
| PIPQQPQQPFPLQ | 6 | 1 | 5 | 5 | 2 | 10′′′ | 9 | 13′ | 4 | 3 | 3 |
| (SEQ ID NO: 130) | | | | | | | | | | | |
| QFIQPEQPFPQQ | 0 | 6 | 28′ | 31′ | 2 | 3 | 8 | 25′′ | 13′ | 2 | 21′′ |
| (SEQ ID NO: 145) | | | | | | | | | | | |
| GQSGYYPTSPQQS | 5 | 2 | 10\hat{ } | 0 | 1 | 4 | 10 | 5 | 4 | 1 | 1 |
| (SEQ ID NO: 49) | | | | | | | | | | | |
| QPFPQPQQPIPVQ | 1 | 12′′ | 40′′ | 51′′ | 4 | 4 | 21 | 37′′ | 30′′ | 2 | 65′′′ |
| (SEQ ID NO: 158) | | | | | | | | | | | |
| TIPEQPEQPFPLQ | 0 | 1 | 14\hat{ } | 224+ | 2 | 4 | 7 | 8 | 5 | 2 | 6 |
| (SEQ ID NO: 165) | | | | | | | | | | | |
| PFPEQPEQPFPQP | 4 | 11′′ | 32′ | 30′ | 5 | 17+ | 8 | 35′′ | 13′ | 0 | 24′′ |
| (SEQ ID NO: 104) | | | | | | | | | | | |
| GIIQPQQPAQL | | | | | 0 | 1 | 7 | 0 | 4 | | 2 |
| (SEQ ID NO: 169) | | | | | | | | | | | |
| FLQPEQPFPEQPEQ | | | | | 1 | 6 | 11 | 18′ | 26′′ | | 23′′ |
| PYPEQPEQPFPQ | | | | | | | | | | | |
| (SEQ ID NO: 170) | | | | | | | | | | | |
| PQQPQQSFPQQQQP | | | | | 11+ | 1 | 4 | 1 | 2 | | 3 |
| A | | | | | | | | | | | |
| (SEQ ID NO: 171) | | | | | | | | | | | |
| GLERPWQEQPLPPQ | 4 | 2 | 0 | 0 | 0 | 16+ | 7 | 22′′ | 1 | 1 | 1 |
| (SEQ ID NO: 37) | | | | | | | | | | | |

~ = Cut-off,
*** = Protein above peptide max,
** = No protein,
* = Hordein peptide response greater than gliadein/gluten peptide response,
H = GLA-DQ2.5 Homozygote.
Significant response as percentage of maximal peptide SFU is shown as follows:
>70% = +,
41-70% = ′′′,
21-40% = ′′,
11-20% = ′,
6-10% = \hat{ }.
Only peptides that reached >70% in at least one individual are shown.

Only peptides that reached >70% in at least one individual are shown.

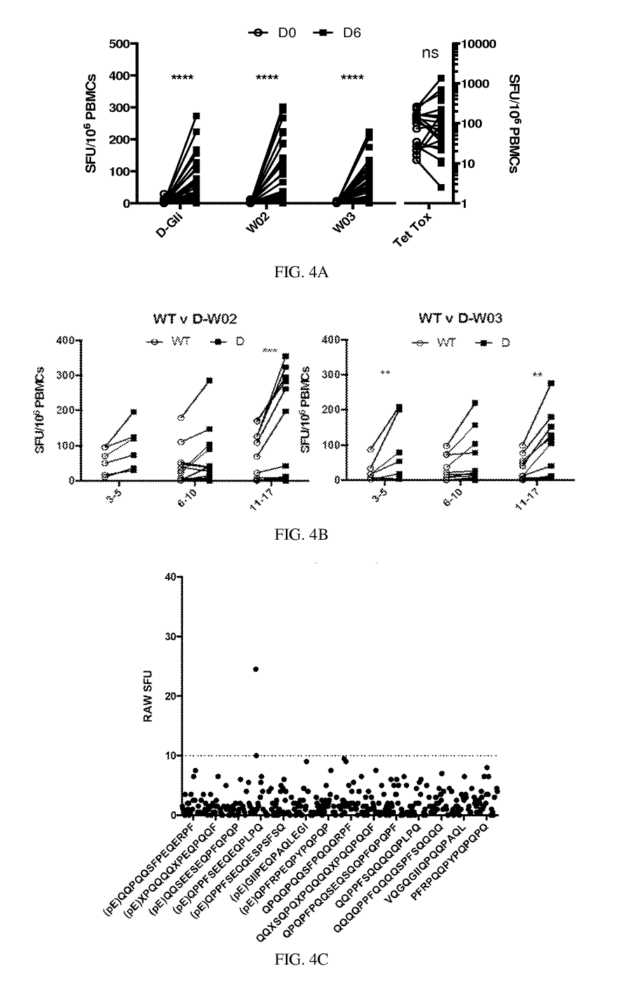

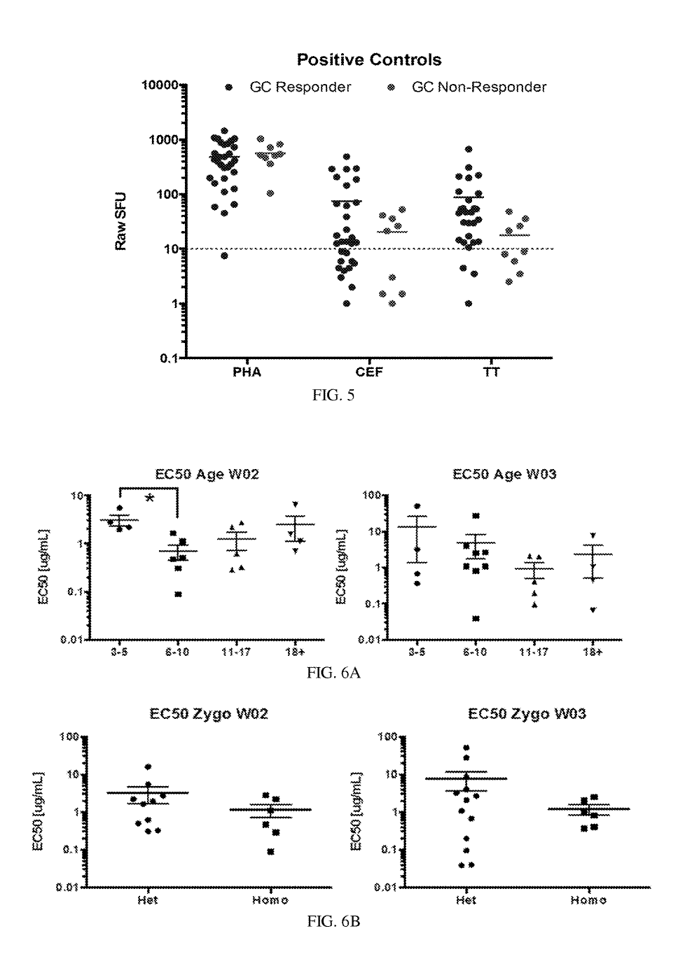

Responses were seen on day 6 following oral wheat challenge, but not prior on day 0 in all but one patient that also had a response above cut-off to deamidated gliadin on day 0 (FIG. 4A; n=34; representative protein and peptide responses shown). There was no difference in tetanus toxoid responses between day 0 and 6. Each age group contained a similar proportion of responders 3-5 yrs, 9/12; 75%; 6-10, 10/12; 83.3%; 11-18 yrs 12/16; 75% (p=0.846 Chi-Square test). One individual in the 11-18 group did not respond to any peptides despite a whole protein response, therefore was not included in the following sections describing peptide responses. There was a clear preference for deamidated antigens including gluten, gliadin and gluten peptides compared to their native counterparts (FIG. 4B and Table 8). The highest IFN-γ ELISpot responses were noted to a gluten peptide compared to whole protein antigen (gliadin or gluten) in the majority of cases (20/31; 64.5%), suggesting that these peptides represented the dominant peptides responsible for most of the immune response to whole protein. In addition, individuals responded to a No. of wheat gluten-derived peptides (Table 8). Responses >70% of the maximal peptide response were seen in the majority of individuals against peptides containing the immuno-dominant DQ2.5-glia-a1/2 w1/w2 epitopes. However, patients also responded to this level to other peptide sequences, but this was generally in a small number of individuals. Non-response to gluten, gliadin and any gluten peptide was significantly associated with a poor response to positive control (FIG. 5; P=0.0035, Chi-square test).

Immunostimulatory peptides were ranked by magnitude of response to establish the hierarchy of gluten peptides following in vivo wheat challenge (Table 8). Dominance scores within each age grouping and overall were calculated (Table 9). 13/70 wheat gluten peptides 2 0 were associated with dominance scores greater or equal to 30 for all age groups. An additional three peptides had scores greater than 30 in the 3-5 yr olds and the 6-10 yrs. Notably, the most dominant three peptides across all age groupings correspond to those observed in adults following wheat challenge (STM), W02 (LQPFPQPELPYPQP, SEQ ID NO: 76) containing DQ2.5-glia-a1a and a2, W01 (LPYPQPELPYPQP, SEQ ID NO: 64) containing DQ2.5-glia-a1b and a2, and W03 (QPFPQPEQPFPWQ, SEQ ID NO: 150) containing DQ2.5-glia-w1 and w2. The list of 13 peptides also contained wild-type versions of W01, W02, and W04 (QPFPQPQQPIPVQ, SEQ ID NO: 158). However, the deamidated equivalents were ranked higher in all cases (Table 9).

TABLE 9
Dominance scores for wheat-derived peptides in CD children
following wheat challenge

| | | 3-5yrs | 6-10yrs | 11-18yrs | Mean |
|---|---|---|---|---|---|
| | Peptide | (n = 9) | (n = 10) | (n = 11) | all ages |
| W02 | LQPFPQPELPYPQPQ | 74.8 | 56.9 | 59.6 | 63.3 |
| | (SEQ ID NO: 7) | | | | |
| W01 | LPYPQPELPYPQP | 68.5 | 71.7 | 51.0 | 62.6 |
| | (SEQ ID NO: 64) | | | | |
| W03 | QPFPQPEQPFPWQP | 66.6 | 51.5 | 46.5 | 54.2 |
| | (SEQ ID NO: 8) | | | | |
| W06 | LQPFPQPELPFPQP | 53.1 | 52.7 | 53.8 | 53.2 |
| | (SEQ ID NO: 70) | | | | |
| W26 | PFPLQPEQPFPQP | 39.4 | 61.6 | 35.0 | 45.8 |
| | (SEQ ID NO: 109) | | | | |
| W04 | QPFPQPEQPIPVQ | 44.5 | 44.2 | 47.5 | 45.6 |
| | (SEQ ID NO: 152) | | | | |
| W08 | LQPFPQPELPYSQP | 42.1 | 51.7 | 40.2 | 44.9 |
| | (SEQ ID NO: 79) | | | | |
| W01 | LPYPQPQLPYPQP | 28.7 | 48.5 | 35.4 | 38.8 |
| WT | (SEQ ID NO: 67) | | | | |
| W02 | LQPFPQPQLPYPQP | 33.1 | 42.9 | 35.4 | 37.7 |
| WT | (SEQ ID NO: 85) | | | | |
| W04 | QPFPQPQQPIPVQ | 25.3 | 43.0 | 30.6 | 34.0 |
| WT | (SEQ ID NO: 158) | | | | |
| W16 | FPQPEQEFPQPQQ | 19.8 | 47.3 | 26.1 | 32.6 |
| | (SEQ ID NO: 31) | | | | |
| W13 | LQPFPQPELPYLQP | 29.3 | 32.0 | 30.1 | 30.7 |
| | (SEQ ID NO: 73) | | | | |
| W32 | PFPEQPEQPFPQP | 26.9 | 34.4 | 28.2 | 30.2 |
| | (SEQ ID NO: 104) | | | | |
| W05 | QPFPQPEQPFSQQ | 23.4 | 33.3 | 27.1 | 28.6 |
| | (SEQ ID NO: 151) | | | | |
| W09 | PQPFLPELPYPQP | 30.7 | 28.6 | 19.1 | 25.2 |
| | (SEQ ID NO: 132) | | | | |
| W07 | QPFPQPEQPFCQQ | 38.2 | 20.5 | 16.2 | 22.7 |
| | (SEQ ID NO: 149) | | | | |

Dominance scores were calculated as the mean percentage of maximal peptide response (from Table 8).

Polyclonal T cell responses after wheat challenge in vivo to the gluten peptides described by Vader et al. were poor with only 2/30; 6.7% having a positive response, and only to the sequence QPPFSEEQEQPLPQ (SEQ ID NO: 172) (FIG. 1C).

### Zygosity Status but Not Age or Time Since Diagnosis Influence the Magnitude of the Gluten-Peptide Specific T Cell Response

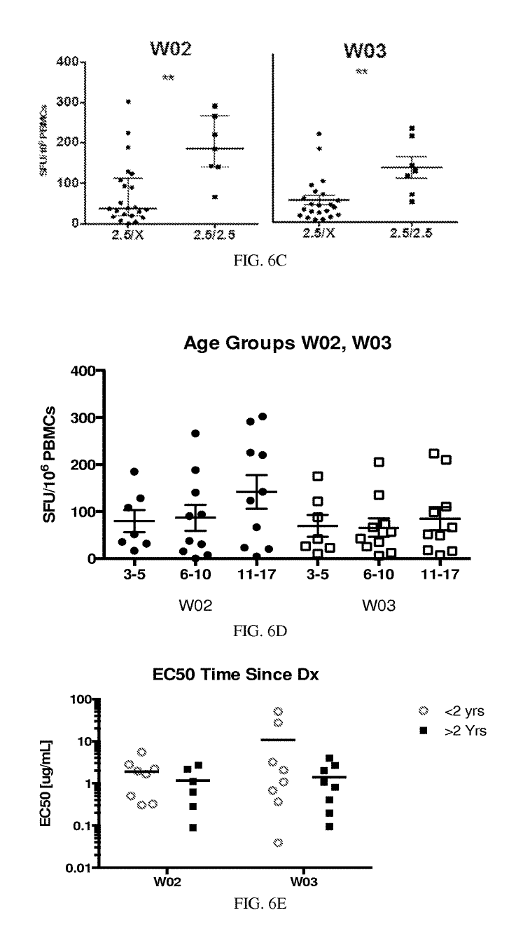

Dose-response curves and EC50 values were calculated in 17 children (n=4 3-5 yrs; n=8 6-10 yrs, and n=5 11-18 yrs) and four adults (18-70 yrs), based on T cell responses to the dominant wheat gluten peptides containing DQ2.5-glia-a1a/a2 (W02) and DQ2.5-glia-w1/w2 (W03; FIG. 6). Median EC50 were similar across each age grouping for the DQ2.5-glia-w1/w2 peptide W03 (FIG. 6A). EC50 values for the DQ2.5-glia-a1a/a2 peptide W02 were similar between 6-10 yrs and 11-18 yrs and adults, but a difference was observed between 3-5 yrs and 6-10 yrs (FIG. 6A). The mean EC50 values were not statistically different between heterozygous and homozygous paediatric individuals for the HLA-DQ2.5 allele (FIG. 6B), but a trend showing lower EC50's in homozygotes was observed. Interestingly, there was a significantly greater magnitude of T cell responses to peptides containing DQ2.5-glia-a1a/a2 and DQ2.5-glia-w1/w2 for participants who were HLA-DQ2.5 homozygous to those heterozygous overall (FIG. 6C), consistent with previous data supporting a gene-dose effect of the DQB1:02 allele. This difference was not seen when comparing age groups (FIG. 6D; p=0.7426, Kruskal-Wallis).

Lastly, EC50 values were compared based on the years since diagnosis of coeliac disease, as this may be an indicator of a more immature immune response. The 3-5 yr age group had a significantly lower time since diagnosis (3-5 yrs 0.4-4.4 yrs; median 1 yr, 6-10 yrs 0.6-7.7 yrs; median 3.3 yrs, and 11-18 yrs 1-9.9 yrs; median 3.6 yrs; p=0.008 Kruskal Wallis). However time since diagnosis did not impact on mean EC50 to either of the two dominant peptides when divided arbitrarily into less than 2 yrs and greater than 2 years (FIG. 6E).

Polyclonal and Clonal Gluten-Specific T Cells Cross-React with Hordein and Secalin Peptides

Next, it was sought to determine if cross-reactivity is a feature of paediatric T cell responses both in vivo and in vitro. Polyclonal T cell responses following oral wheat challenge cross-reacted to a series of peptides derived from barley hordein and rye secalin (Table 10; n=22). Fifteen of twenty-two (68.2%) CD children responded to the peptide W03 containing the immunodominant wheat T cell epitopes DQ2.5-glia-w1/w2 and epitope DQ2.5-hor-1 (same epitope sequence, different grain; Table 10), and in most cases also responded to other peptides containing homologous sequences within hordein and secalin.

TABLE 10
Cross-reactive T cell responses in paediatric CD following wheat challenge

| | 3-5yrs | | | 6-10yrs | | | | | | | |
|---|---|---|---|---|---|---|---|---|---|---|---|
| | AA | HH | GG | I | J | K | V | W | Y | JJ | MM |
| Wheat | | | | | | | | | | | |
| QPFPQPEQPFPWQP | 52.5+ | 10+ | 14.5+ | 5.5 | 95- | 18.5′′ | 25+ | 9 | 10- | 40.5+30 | 11′′ |
| (SEQ ID NO: 8) | | | | | | | | | | | |
| Barley | | | | | | | | | | | |
| QPFPQPEQPFPLQ | 65+ | 12+ | 13+ | 13+ | 88′′′ | 29′′′ | 22′′′ | 8 | 9 | 45+ | 6 |
| (SEQ ID NO: 74) | | | | | | | | | | | |
| NPLQPEQPFPLQPQPP | 10′ | 13+ | 11+ | 1 | 38′ | 19′′ | 6 | 7 | 3 | 14′′ | 4 |
| (SEQ ID NO: 44) | | | | | | | | | | | |
| QPFPQPEQPIPYQ | 31′′′ | 11+ | 9 | 5 | 124′′′ | 21′′ | 22′′′ | 11 | 11′′′ | 30′′′ | 8 |
| (SEQ ID NO: 73) | | | | | | | | | | | |
| PEQPFPEQPQPYPQQP | 1 | 5 | 12+ | 2 | 36′ | 6 | 1 | 9 | 1 | 1 | 4 |
| (SEQ ID NO: 47) | | | | | | | | | | | |
| PEQPFQPEQPFPQQ | 6 | 2 | 12+ | 1 | 31′ | 8 | 7 | 3 | 1 | 3 | 7 |
| (SEQ ID NO: 50) | | | | | | | | | | | |
| PEQPQPFPEQPVPQQP | 3 | 2 | 11+ | 0 | 7 | 4 | 3 | 4 | 2 | 0 | 4 |
| (SEQ ID NO: 53) | | | | | | | | | | | |
| QPFPQPEQPFSWQ | 26′′ | 8 | 0 | 2 | 79′′′ | 20′′ | 25+ | 3 | 8 | 42+ | 6 |
| (SEQ ID NO: 80) | | | | | | | | | | | |
| QPFPQPEQPFRQQ | 24′′ | 5 | 6 | 4 | 15\hat{ } | 21′′ | 20′′′ | 8 | 8 | 37+ | 6 |
| (SEQ ID NO: 77) | | | | | | | | | | | |
| QEFPQPEQPFPQQ | 30′′′ | 6 | 8 | 8 | 93′′′ | 21′′ | 11′′ | 3 | 2 | 36+ | 2 |
| (SEQ ID NO: 68) | | | | | | | | | | | |
| Rye | | | | | | | | | | | |
| QPFPQPEQPIPQQ | 24′′ | 12+ | 4 | 3 | 132′′′ | 11′ | 17′′′ | 5 | 7 | 44+ | 7 |
| (SEQ ID NO: 100) | | | | | | | | | | | |
| QPFPQPEQPTPIQ | 20′′ | 12+ | 8 | 0 | 86′′′ | 7 | 5 | 0 | 4 | 38+ | 6 |
| (SEQ ID NO: 103) | | | | | | | | | | | |
| QPFPQPEQQLPLQ | 2 | 11+ | 6 | 3 | 79′′′ | 9 | 3 | 3 | 5 | 11′′ | 11 |
| (SEQ ID NO: 106) | | | | | | | | | | | |
| QPFPQPEQELPLQ | 9 | 5 | 15+ | 6 | 57′′ | 13′ | 7 | 0 | 0 | 33 | 8 |
| (SEQ ID NO: 96) | | | | | | | | | | | |
| QLFPLPEQPFPQP | 3 | 0 | 14+ | 1 | 26′ | 5 | 4 | 4 | 2 | 7 | 1 |
| (SEQ ID NO: 90) | | | | | | | | | | | |
| FPQTEQPEQPFPQP | 3 | 4 | 13+ | 0 | 67′′ | 19′′ | 6 | 5 | 3 | 5 | 5 |
| (SEQ ID NO: 39) | | | | | | | | | | | |
| QPFPQPEQPFPQS | 44′′′ | 7 | 12+ | 2 | 57′′ | 18′′ | 25+ | 6 | 4 | 32′′′ | 11′′′ |
| (SEQ ID NO: 98) | | | | | | | | | | | |
| PFPLQPEQPVPEQPQ | 5 | 3 | 11+ | 1 | 3 | 5 | 7 | 11 | 2 | 1 | 1 |
| (SEQ ID NO: 84) | | | | | | | | | | | |
| LPFPQPEQPFVVV | 18′′ | 8 | 8 | 7 | 29′ | 18′′ | 13′′ | 9 | 7 | 34+ | 3 |
| (SEQ ID NO: 48) | | | | | | | | | | | |
| QPEQPFPLQPEQPVP | 6 | 2 | 8 | 1 | 11\hat{ } | 8 | 4 | 4 | 1 | 3 | 19+ |
| (SEQ ID NO: 93) | | | | | | | | | | | |
| | 11-17yrs | | | | | | | | | | |
| | A | D | F | E | G | H | P | Q | U | T | NN |
| Wheat | | | | | | | | | | | |
| QPFPQPEQPFPWQP (SEQ | 15 | 30.5+ | 845′′′ | 44′ | 8 | 8.5 | 20 | 63′′′ | 17′′ | 67.5 | 15 |
| ID NO: 8) | | | | | | | | | | | |
| Barley | | | | | | | | | | | |
| QPFPQPEQPFPLQ (SEQ | 5 | 16′′′ | 64′′ | 70′′ | 10′′′ | 4 | 21 | 83+ | 18′′ | 94+ | 0 |
| ID NO: 74) | | | | | | | | | | | |
| NPLQPEQPFPLQPQPP | 3 | 12′′ | 25′ | 21\hat{ } | 1 | 14+ | 6 | 23′′ | 9 | 33′′ | 1 |
| (SEQ ID NO: 44) | | | | | | | | | | | |
| QPFPQPEQPIPYQ (SEQ | 8 | 23+ | 84′′′ | 86′′ | 2 | 3 | 33′′′ | 58′′′ | 55′′′ | 84+ | 0 |
| ID NO: 73) | | | | | | | | | | | |
| PEQPFPEQPQPYPQQP | 2 | 2 | 6 | 13\hat{ } | 2 | 6 | 10 | 2 | 1 | 0 | 1 |
| (SEQ ID NO: 47) | | | | | | | | | | | |
| PEQPFQPEQPFPQQ (SEQ | 2 | 1 | 6 | 12\hat{ } | 5 | 3 | 9 | 7 | 6 | 11′ | 0 |
| ID NO: 50) | | | | | | | | | | | |
| PEQPQPFPEQPVPQQP | 3 | 3 | 6 | 4 | 1 | 0 | 8 | 6 | 1 | 5 | 0 |
| (SEQ ID NO: 53) | | | | | | | | | | | |
| QPFPQPEQPFSWQ (SEQ | 0 | 16′′′ | 19′ | 34′ | 6 | 1 | 14 | 29′′ | 18′′ | 41′′′ | 1 |
| ID NO: 80) | | | | | | | | | | | |
| QPFPQPEQPFRQQ (SEQ | 1 | 15′′′ | 21′ | 37′ | 3 | 0 | 16 | 44′′′ | 21′′ | 58′′′ | 0 |
| ID NO: 77) | | | | | | | | | | | |
| QEFPQPEQPFPQQ (SEQ | 4 | 15′′′ | 38′′ | 30′ | 4 | 1 | 7 | 44′′′ | 23′′ | 67+ | 1 |
| ID NO: 68) | | | | | | | | | | | |
| Rye | | | | | | | | | | | |
| QPFPQPEQPIPQQ (SEQ | 3 | 29+ | 62′′ | 87′′ | 4 | 1 | 48+ | 64′′′ | 41′′′ | 87+ | 1 |
| ID NO: 100) | | | | | | | | | | | |
| QPFPQPEQPTPIQ (SEQ | 6 | 11′′ | 73 | 63′′ | 1 | 1 | 23′′ | 39′′′ | 21′′ | 61′′′ | 2 |
| ID NO: 103) | | | | | | | | | | | |
| QPFPQPEQQLPLQ (SEQ | 1 | 5 | 5 | 30′ | 1 | 1 | 11 | 11′ | 4 | 27′′ | 3 |
| ID NO: 106) | | | | | | | | | | | |
| QPFPQPEQELPLQ (SEQ | 4 | 7 | 31′ | 46′′ | 1 | 5 | 17 | 14′ | 12′ | 36′′ | 1 |
| ID NO: 96) | | | | | | | | | | | |
| QLFPLPEQPFPQP (SEQ | 2 | 2 | 2 | 2 | 0 | 0 | 11 | 13′ | 9 | 13′ | 1 |
| ID NO: 90) | | | | | | | | | | | |
| FPQTEQPEQPFPQP | 1 | 0 | 2 | 3 | 2 | 1 | 3 | 10′ | 5 | 7 | 0 |
| (SEQ ID NO: 39) | | | | | | | | | | | |
| QPFPQPEQPFPQS | 4 | 15′′′ | 35′ | 46′′ | 5 | 4 | 13 | 55′′′ | 27′′ | 70+ | 1 |
| (SEQ ID NO: 98) | | | | | | | | | | | |
| PFPLQPEQPVPEQPQ | 2 | 9 | 20′ | 3 | 0 | 3 | 6 | 5 | 10′ | 21′′ | 1 |
| (SEQ ID NO: 84) | | | | | | | | | | | |
| LPFPQPEQPFVVV (SEQ | 1 | 20′′′ | 15\hat{ } | 16\hat{ } | 3 | 3 | 20 | 31′′ | 8 | 42′′′ | 1 |
| ID NO: 48) | | | | | | | | | | | |
| QPEQPFPLQPEQPVP | 1 | 0 | 27′ | 9 | 2 | 3 | 12 | 20′′ | 9 | 16′ | 0 |
| (SEQ ID NO: 93) | | | | | | | | | | | |

Significant response as a percentage of maximal peptide SFU is shown as follows: >70% = +,
41-70% = ′′′,
21-40% = ′′,
11-20% = ′,
6-10% - \hat{ }.
Only peptides that reached >70 % in at least one individual are shown.

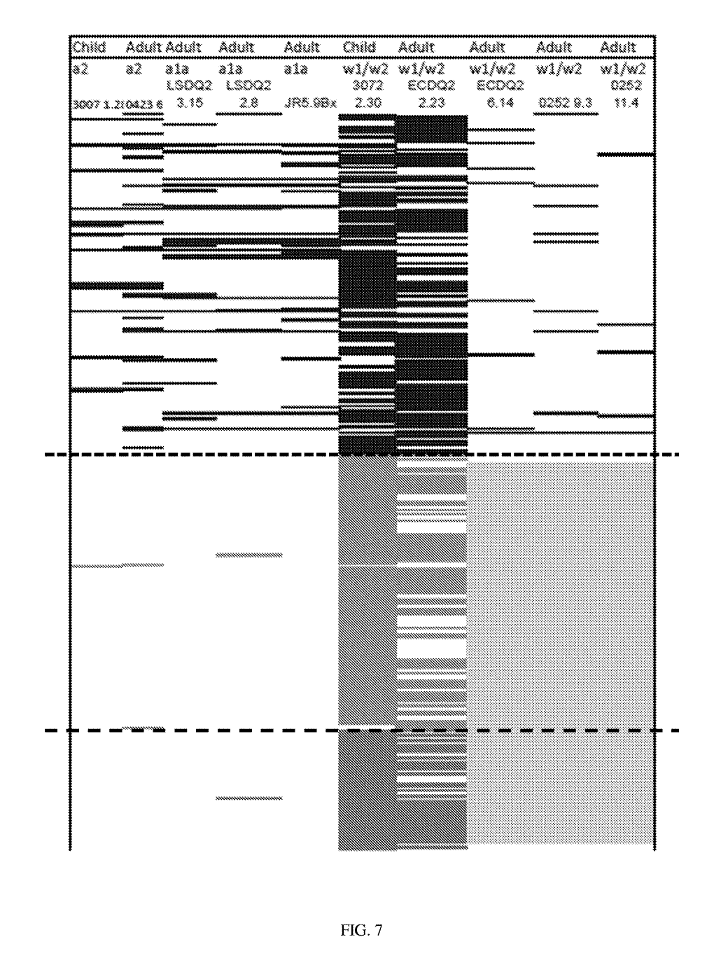

Three TCC specific to DQ2.5-glia-a2 from wheat alpha-gliadin and DQ2-ω-I/-II, from wheat omega gliadin were raised from two children with CD and their recognition of comprehensive wheat gliadin, barley hordein and rye secalin peptide libraries assessed (FIG. 7). TCC 3007.28 (specific to DQ2.5-glia-a2) showed minimal reactivity to hordein or secalin peptides, consistent with the observation that peptides containing the dominant wheat T cell epitopes DQ2.5-glia-a1a and DQ2.5-glia-a2 are infrequent in barley or rye (FIG. 7). In contrast, TCC specific to DQ2.5-glia-ω-I/-II showed substantially more immunoreactivity to a range of hordein and secalin peptides that encompass both epitopes. When compared to adult clones specific for the same epitopes, reactivity patterns were very similar (FIG. 7). Collectively these findings support the high level of cross-reactivity by polyclonal T cells induced by wheat gluten challenge, and TCCs specific for dominant wheat gluten peptides, for hordein and secalin as previously observed in adults with CD [ref. 11].

Symptoms were Variable Following Wheat Challenge and Did Not Correlate with T Cell Responses

28/40 (70%) of all participants were symptomatic following 3-day wheat challenge but the majority were able to complete all 3-days of wheat ingestion (Table 6). The proportion of symptomatic individuals did not vary by age grouping, irrespective of symptom severity. HLA-DQ2.5 homozygosity was not associated with a higher proportion of symptomatic responders at any severity level. Three of twelve patients within the 3-5 yr, 1/12 6-10 yrs, and 2/16 11-18 yrs age group vomited following gluten challenge. In a post-hoc analysis, the presence of symptoms did not correlate with the presence of a positive immune response to gluten or gluten peptide (p=0.6538 Chi-square test).

### Discussion

There is a substantial health burden imposed on patients with chronic autoimmune disease that might be overcome if relevant antigenic targets were identified, allowing development of antigen-specific therapeutics or prophylactics. CD is a prototype autoimmune illness to assess the specificity of the immune response as the driving antigen, gluten, is known, and an immune recall response can be assessed following short-term oral gluten challenge. While the gluten-specific immune response in adults with CD has been comprehensively characterized, immune responses mounted by children with CD have been the subject of few studies to date, and these are limited to long-term culture derived T cell lines and proliferation assays.

Vader et al. observed responses to the a2/a9 peptides in half of the paediatric group tested and showed T cell lines isolated from paediatric CD patients responded to a diverse repertoire of peptides including glutenin sequences that did not require deamidation. However, there is substantial inconsistency in recognition of these epitopes between T cell lines from adult Norwegian CD donors (17/17, 100%) 21, compared to Dutch children (8/16, 50%) and adults (2/4) [ref. 15]. It is unclear if this reflects differences between CD in Norway and the Netherlands, adults and children, or simply differences in methodology [ref. 15]. Norwegian researchers acknowledge that in vitro culture of T cell lines and clones “may alter the composition and function of the T cell population of interest and may favor the growth of certain subpopulations” [ref. 22].

Other groups have utilized a seven day proliferation assay performed on PBMC collected from newly diagnosed children in order to measure gluten-specific T cell responses [refs. 15-17]. Recently Liu et al. were able to detect proliferation in response to a peptide containing a deamidation variant of DQ8-glia-a1 in 60% of CD children even those not carrying HLA-DQ8. Responses to DQ2.5-glia a1a and a2 were seen in 3/10 and 0/10 individual CD children respectively. Lammi et al. detected CD4+ T cell proliferation in response to tTG-treated gliadin in 11/20 untreated CD children but no proliferation in 15 CD children in response to peptides containing DQ2.5-glia-a1a/1b/2 [ref. 16]. Again, it is unclear how much methodological limitations caused by prolonged culture driven by mitogens have hampered the detection of true gluten peptide specific responses that are relevant in vivo.

In contrast, the use of the approach described herein to assess polyclonal T cells induced by short-term gluten challenge indicates remarkable consistency in T cell recognition of DQ2.5-glia-α1/2 in HLA DQ2+ adults with CD in England, Italy, Norway, and Australia [refs. 7,9,22]. In this study, deamidation enhanced the in vivo T cell response to immunodominant gliadin-derived peptides across all age groups. Notably, responses to the peptides described by Vader et al. (including two glutenin-derived peptides) were low or non-existent after wheat ingestion.

HLA DQB1*02 gene dose is reported to increase the risk of developing CD [ref. 23], and homozygosity for DQB1*02 is associated with a younger age at diagnosis, more severe symptoms and slower recovery after commencing a gluten-free diet (GFD) [ref. 24]. Whether HLA DQ2 homozygosity is over-represented in children with CD compared to adults is unclear. The data provided herein supported a gene-dose effect, as EC50's were generally lower for homozygotes and T cell response magnitude was higher. The range of specificity toward gliadin, hordein, and secalin-derived peptides was expanded in homozygotes compared with heterozygotes, most likely due to additional presentation of these peptides on the surface of APC.

The study herein aimed to capture individuals in the earliest stage of disease progression. However, it is impossible to accurately establish disease duration because of clinical heterogeneity. Thus while it is not possible to assess the primary T-cell response to gluten, oral gluten challenge and isolating T cells from blood or intestinal biopsies allows the recall response against gluten to be compared between adults and children of any age. The results herein clearly suggest that although younger and more likely to be at an earlier stage of disease evolution and duration compared to adults with long-standing CD, recall T cell responses in children with CD shared many features of those in adults. Most importantly, the study herein showed consistency in the hierarchy of the dominant gluten peptides compared to adults, and that responses were frequently greater than to whole antigen, suggesting that the contribution of other untested gluten peptides was likely to be minimal. T cell responses in those less than two years since diagnosis were compared to those that had been diagnosed over two years and no differences were observed. This suggests that the affinity maturation of the T cell response may have already occurred even before disease diagnosis.

The study herein describes the investigation of the in vivo polyclonal T cell response to gluten and key immunostimulatory gluten peptides in HLA-DQ2.5+ children aged 3 to 17 with CD. The findings support the consistency of the recall immune response to key dominant peptides, and supports the feasibility of peptide-based applications designed in adults with CD for children with CD.

### REFERENCES

1. Anderson, W. H. & Mackay, I. R. Gut reactions-from celiac affection to autoimmune model. N Engl J Med 371, 6-7 (2014).
2. Husby, S., et al. European Society for Pediatric Gastroenterology, Hepatology, and Nutrition guidelines for the diagnosis of coeliac disease. J Pediatr Gastroenterol Nutr 54, 136-160 (2012).
3. Liu, E., et al. Risk of pediatric celiac disease according to HLA haplotype and country. N Engl J Med 371, 42-49 (2014).
4. Lionetti, E., et al. Introduction of gluten, HLA status, and the risk of celiac disease in children. N Engl J Med 371, 1295-1303 (2014).
5. Abadie, V., Sollid, L. M., Barreiro, L. B. & Jabri, B. Integration of genetic and immunological insights into a model of celiac disease pathogenesis. Annu Rev Immunol 29, 493-525 (2011).
6. Vader, W., et al. The HLA-DQ2 gene dose effect in celiac disease is directly related to the magnitude and breadth of gluten-specific T cell responses. Proceedings of the National Academy of Sciences of the United States of America 100, 12390-12395 (2003).
7. Anderson, R. P., Degano, P., Godkin, A. J., Jewell, D. P. & Hill, A. V. In vivo antigen challenge in celiac disease identifies a single transglutaminase-modified peptide as the dominant A-gliadin T-cell epitope. Nature Medicine 6, 337-342 (2000).
8. Anderson, R. P., et al. T cells in peripheral blood after gluten challenge in coeliac disease. Gut 54, 1217-1223 (2005).
9. Camarca, A., et al. Intestinal T cell responses to gluten peptides are largely heterogeneous: implications for a peptide-based therapy in celiac disease. J Immunol 182, 4158-4166 (2009).
10. Camarca, A., et al. Short wheat challenge is a reproducible in-vivo assay to detect immune response to gluten. Clin Exp Immunol 169, 129-136 (2012).
11. Tye-Din, J. A., et al. Comprehensive, quantitative mapping of T cell epitopes in gluten in celiac disease. Sci Transl Med 2, 41ra51 (2010).
12. Petersen, J., et al. T-cell receptor recognition of HLA-DQ2-gliadin complexes 2 5 associated with celiac disease. Nature structural & molecular biology 21, 480-488 (2014).
13. Qiao, S. W., et al. Posttranslational modification of gluten shapes TCR usage in celiac disease. J Immunol 187, 3064-3071 (2011).
14. Qiao, S. W., Christophersen, A., Lundin, K. E. & Sollid, L. M. Biased usage and preferred pairing of alpha- and beta-chains of TCRs specific for an immunodominant gluten epitope in coeliac disease. Int Immunol 26, 13-19 (2014).
15. Vader, W., et al. The gluten response in children with celiac disease is directed toward multiple gliadin and glutenin peptides. Gastroenterology 122, 1729-1737 (2002).
16. Lammi, A., Arikoski, P., Vaarala, O., Kinnunen, T. & Ilonen, J. Increased peripheral blood CD4+ T cell responses to deamidated but not to native gliadin in children with coeliac disease. Clin Exp Immunol 168, 207-214 (2012).
17. Liu, E., et al. Exploring T cell reactivity to gliadin in young children with newly diagnosed celiac disease. Autoimmune diseases 2014, 927190 (2014).
18. Walker-Smith, J., Guandalini, S., Schmitz, J., Shmerling, D. & Visakorpi, J. Revised criteria for diagnosis of coeliac disease. Report of Working Group of European Society of Paediatric Gastroenterology and Nutrition. Archives of Disease in Childhood 65, 909-911 (1990).
19. Hardy, M. Y., et al. Ingestion of oats and barley in patients with celiac disease mobilizes cross-reactive T cells activated by avenin peptides and immuno-dominant hordein peptides. J Autoimmun (2014).
20. Mannering, S. I., et al. An efficient method for cloning human autoantigen-specific T cells. J Immunol Methods 298, 83-92 (2005).
21. Arentz-Hansen, H., et al. The intestinal T cell response to alpha-gliadin in adult celiac disease is focused on a single deamidated glutamine targeted by tissue transglutaminase. Journal of Experimental Medicine 191, 603-612 (2000).
22. Raki, M., et al. Tetramer visualization of gut-homing gluten-specific T cells in the peripheral blood of celiac disease patients. Proceedings of the National Academy of Sciences of the United States of America 104, 2831-2836 (2007).
23. Mearin, M. L., et al. HLA-DR phenotypes in Spanish coeliac children: their contribution to the understanding of the genetics of the disease. Gut 24, 532-537 (1983).
24. Karinen, H., et al. Gene dose effect of the DQB1*0201 allele contributes to severity of coeliac disease. Scand J Gastroenterol 41, 191-199 (2006).

### Example 6

### The specificity and Dominance of the Polyclonal T Cell Response to Gluten After Wheat Ingestion in Celiac Disease is Consistent Over Time

The study from Example 5 was further investigated with a slightly different patient population that included 41 pediatric CD volunteers (aged 3-17; median 9 yrs; 17 M:24 F). The further investigation is described below.

### Abstract

Antigen-specific approaches for the diagnosis and treatment of celiac disease (CD) require detailed understanding of the specificity of pathogenic T cells for gluten. The peptides responsible for this response are well established in adults with longstanding CD, but studies utilizing cultured T cells lines and clones from children suggest that the T cell response nearer disease onset fundamentally differs in terms of the diversity and hierarchy of gluten peptides implicated. The present study aimed to characterize the in vivo recall T cell response in blood following oral wheat gluten challenge in pediatric CD using fresh peripheral blood mononuclear cells, and cultured T cell lines and clones, and a targeted screen of immunogenic peptides from wheat. Gluten-specific responses were detected in 30 of 41 (73%) CD children aged 3-17 years. Recognition of peptides immunogenic in adults with CD was highly consistent and deamidation was important for bioactivity. Age or time since diagnosis did not affect the magnitude of T cell responses to dominant peptides. T cell clones (TCC) raised from CD children specific for dominant α- or ω-gliadin peptides demonstrated comparable patterns of cross-reactivity to wheat, rye, and barley peptide libraries as TCC from CD adults. Similarities in the nature of the T cells induced by in vivo wheat challenge in pediatric CD indicates that peptide-based applications designed in adults are likely to be applicable in children.

### Introduction

Detailed understanding of the function and specificity of T cells responsible for autoimmunity is widely expected to translate to more effective strategies to diagnose, treat and prevent autoimmune disease (1). Amongst all the autoimmune diseases that affect humans, celiac disease (CD) is the only one for which there is currently broad consensus regarding the identity of immunodominant epitopes consistently recognized by pathogenic CD4+ T cells (2). However, little is known of the CD4+ T-cell response in children, close to the time when the immunological events responsible for CD are initiated (3). Inadequate understanding of these early events is concerning given the steadily rising incidence of CD (4, 5), and recent failures of large intervention studies aimed at reducing the development of CD in high-risk infants (6, 7).

CD is manifest by gluten-dependent intestinal damage and IgA autoantibody specific for transglutaminase type 2 (tTG) (8). Almost all patients meeting the diagnostic criteria for CD possess the MHC Class II HLA (human leukocyte antigen) genes HLA-DQB1*02 and HLA-DQA1*05 or HLA-DQA1*02, or HLA-DQA1*03 and HLA-DQB1*03:02, which encode the functional heterodimers HLA-DQ2.5, HLA-DQ2.2, and HLA-DQ8 responsible for presenting gluten-derived peptides to recognized by CD4+ T cells in CD (9).

There is compelling evidence that CD4+ T cells isolated from intestinal tissue or circulating in blood at increased frequencies after oral wheat challenge in adult CD patients preferentially recognize deamidated gluten peptides that include highly conserved epitopes (10, 11). Approximately half or more of the wheat gluten-reactive CD4+ T cells expanded from intestinal tissue or circulating after oral wheat challenge in HLA-DQ2.5+CD patients recognize one of two overlapping epitopes derived from partially deamidated wheat α-gliadin (PFPQPELPY: DQ2.5-glia-α1 or PQPELPYPQ: DQ2.5-glia-α2α) (12, 13). Several other epitopes, including two from partially deamidated w-gliadin (PFPQPEQPF: DQ2.5-glia-ω1 and PQPEQPFPW: DQ2.5-glia-ω2), are also commonly recognized by a substantial proportion of wheat gluten-reactive T cells from the intestinal mucosa and in blood after oral wheat challenge in adult CD (14, 15).

Despite most patients diagnosed with CD being in adulthood, prospective observational studies indicate the onset of CD is typically between one and six-years of age (3). Many features of the immunopathology of CD are common to adults and children, for example, chronic ingestion of gluten is associated with elevated levels of serum IgA specific tTG and both IgG and IgA specific for highly conserved deamidated 5 mer peptide motifs derived from gliadin (16-19). However, the only detailed study to address the specificity of the T-cell response to gluten in children concluded there were fundamental differences from that in adults (20). Vader et al. assessed the specificity of T cell lines and clones derived from intestinal biopsies collected from children aged between one and 12 years old (average age 4.0 years) when they were diagnosed with CD. Far greater diversity of the gluten-specific T-cell response was observed than had previously been appreciated, and included native gluten peptides not dependent on deamidation. Vader et al. went on to propose that although deamidation may not be required to initiate gluten-specific T cell responses, it is likely to occur because of elevated levels of tTG in the inflamed mucosa, and this could facilitate epitope spreading.

Despite its potential significance, there have been no further studies to test whether the T cell response to gluten is more diverse in children than in adults with CD. The aim of the present study was to determine the hierarchy of gluten peptides responsible for activating T cells freshly isolated from blood after oral wheat challenge in children with CD, and determine if T-cell recognition of gluten peptide differed between children and adults. For the first time, this study establishes the in vivo specificity and hierarchy of the polyclonal T cell response to gluten in HLA-DQ2.5+ children with CD, and determines the redundancy of peptide recognition to enable definitive comparisons on the specificity, magnitude, maturity and clonality of T cell responses to gluten in children and adults.

### Results

Clinical Response to Short-Term Oral Wheat Challenge in Children with CD

Forty-one Australian children (aged 3-17; 17 M:24 F) with a median age of CD diagnosis of 5 yrs (67 months, range 19-187) participated (Table 12). Each undertook a wheat challenge on a single occasion consuming wheat bread for up to 3-days (median age 9 years; 117 months, range 38-210) (Table 13). Most volunteers (31/41) had normal baseline tTG-IgA and DGP-IgG levels immediately prior to undertaking the challenge. The wheat challenge induced symptoms in 29/41 (70.7%) volunteers that were mainly gastrointestinal (such as nausea, bloating, abdominal pain and vomiting), comparable to that experienced by CD adults after short-term wheat challenge (21). All symptoms resolved with expectant management. Four volunteers (3-5 yrs n=1, 6-10 yrs n=2, 11-18 yrs n=1) did not complete the full 3-day challenge due to vomiting (n=3) or poor tolerability (n=1), but D6 blood was still collected and analysed in all. Symptoms had fully resolved in 26/29 (90%) of children by D6. The proportion of symptomatic individuals did not vary by age grouping (p=NS, Chi-square test) or by HLA-DQ2.5 zygosity (p=NS, Fisher's exact test). The presence of symptoms did not correlate with a significant T cell response (see below) to gliadin, gluten or any gluten peptide (p=NS, Chi-square test) although all volunteers who experienced vomiting mounted a positive response to gluten peptide(s). There was no difference in age or degree of villous atrophy (Marsh 3A, 3B or 3C) at diagnosis of CD between HLA-DQ2.5 homozygous or heterozygous volunteers (p=NS, Chi square test).

TABLE 12
Participant clinical details at diagnosis.

| | Age at | | | | | | | |
|---|---|---|---|---|---|---|---|---|
| | diagnosis | | DQ2.5 | Marsh | tTG- | DGP- | | Clinical |
| Subject | Yr (mths) | Sex | zygosity | score | IgA | IgG | AGA | presentation |
| C1 | 2 (22) | F | 2.5/x | 3B | 95^{b} | NA | 170^{a} | B, FH |
| C2 | 3 (41) | F | 2.5/2.5 | 3C | >100^{b} | NA | 208^{a} | B |
| C3 | 4 (40) | F | 2.5/x | 3B | >100^{b} | >100^{b} | NA | B, An |
| C4 | 4 (39) | F | 2.5/x | 3C | >128^{a} | 114^{a} | NA | D, V |
| C5 | 4 (44) | M | 2.5/x | 3B | >128^{a} | 17^{a} | NA | F, FTT |
| C6 | 5 (62) | M | 2.5/x | 3A | 81^{b} | 63^{b} | NA | B, D |
| C7 | 4 (48) | M | 2.5/x | 3C | <5^{b} | 132^{b} | NA | B, D, An, pale |
| C8 | 4 (50) | F | 2.5/x | 3B | >200^{c} | 120^{c} | NA | P, L |
| C9 | 5 (59) | F | 2.5/2.5 | 3C | >128^{a} | 151^{a} | NA | L, FH |
| C10 | 5 (56) | F | 2.5/x | 3B | 60^{a} | 49^{a} | NA | FH, A |
| C11 | 5 (67) | F | 2.5/x | 3B | >100^{d} | >100^{b} | NA | FH, A |
| C12 | 2 (19) | F | 2.5/x | 3B | >100^{b} | >100^{b} | NA | B, I, weight loss |
| C13 | 5 (59) | M | 2.5/2.5 | 3B | 19^{b} | >100^{b} | NA | L, P |
| C14 | 3 (36) | F | 2.5/2.5 | 3B | >100^{e} | >100^{e} | NA | Iron, B |
| C15 | 5 (65) | M | 2.5/x | 3B | 100^{b} | >100^{b} | NA | FH, A |
| C16 | 6 (76) | F | 2.5/x | 3B | >100^{b} | 40^{b} | NA | FH, A |
| C17 | 4 (55) | F | 2.5/x | 3C | >200^{e} | NA | NA | V, D |
| C18 | 5 (57) | F | 2.5/x | 3B | >100^{e} | 4^{e} | NA | FH, A |
| C19 | 7 (79) | F | 2.5/x | 3B | >200^{e} | >200^{e} | NA | C, An |
| C20 | 2 (25) | F | 2.5/2.5 | 3A | >100^{e} | NA | NA | P, FH |
| C21 | 8 (91) | F | 2.5/x | 3A | 100^{c} | 17^{b} | NA | C, B |
| C22 | 6 (74) | M | 2.5/x | 3B | >100^{b} | >100^{d} | NA | D |
| C23 | 10 (121) | F | 2.5/x | 3C | 175^{b} | 193^{b} | NA | L, I |
| C24 | 7 (87) | M | 2.5/x | 3C | Pos^{e} | Pos^{e} | NA | L, I |
| C25 | 5 (58) | F | 2.5/x | 3C | >100^{c} | >100^{e} | NA | L, P, B, C |
| C26 | 2 (35) | M | 2.5/2.5 | 3B | 92^{b} | 75^{d} | NA | D, I |
| C27 | 9 (116) | F | 2.5/2.5 | 3B | >100^{c} | >100^{b} | NA | P, D, B |
| C28 | 11 (129) | M | 2.5/x | 3A | >200^{c} | 167^{c} | NA | Headaches |
| C29 | 11 (126) | F | 2.5/x | 3B | 138^{a} | 47^{a} | NA | L |
| C30 | 4 (45) | F | 2.5/2.5 | 3B | Pos^{e} | NA | NA | P, rash |
| C31 | 10 (119) | F | 2.5/x | 3A | >100^{e} | NA | NA | P |
| C32 | 11 (138) | F | 2.5/x | 3A | >128^{a} | 38^{b} | NA | P |
| C33 | 10 (116) | M | 2.5/x | 3B | >100^{c} | 40^{b} | NA | F, C, B, L |
| C34 | 11 (133) | M | 2.5/2.5 | 3C | >100^{e} | >100^{e} | NA | FH |
| C35 | 10 (118) | M | 2.5/x | 3C | 173^{c} | NA | 127^{b} | C, F |
| C36 | 15 (175) | F | 2.5/x | 3B | >100^{d} | NA | >100^{a} | L, Iron |
| C37 | 14 (176) | M | 2.5/2.5 | 3C | 27^{b} | 14^{e} | NA | I, headaches |
| C38 | 15 (179) | M | 2.5/2.5 | 3C | Pos^{e} | Pos^{e} | NA | Poor growth |
| C39 | 15 (187) | M | 2.5/x | 3C | 84^{b} | NA | 77^{a} | FH, L, Iron |
| C40 | 10 (123) | M | 2.5/x | 3C | 175^{c} | 23^{b} | NA | D, C, I |
| C41 | 13 (153) | M | 2.5/2.5 | 3B | NA | >100^{e} | NA | FHx, A |

x denotes a haplotype other than HLA-DQ2.5 or HLA-DQ8.
tTG (tissue transglutaminase),
DGP (deamidated gliadin peptide),
AGA (anti-gliadin antibody).
Normal serology ranges: ^{a}tTG-IgA (0-6), DGP-IgG (0-6), and AGA (<20); ^{b}tTG-IgA (<5), DGP-IgG (<20), and AGA (<46); ^{c}tTG-IgA (<20) and DGP-IgG (<25); ^{d}tTG-IgA(<4) and DGP-IgG (<46); ^{e}tTG-IgA (<7) and DGP-IgG (<5);
^{e}Reference range not determined.
Pos = positive but no value recorded.
NA = Not applicable/performed.
A = asymptomatic,
An = anorexia/poor appetite,
B = bloating/distension,
C = constipation,
D = diarrhoea,
F = flatulence,
FH = family history of CD,
FTT = failure to thrive,
I = irritable/moody,
Iron = iron deficiency,
P = abdominal pain,
L = lethargy,
V = vomiting.

TABLE 13
Gluten challenge details.

| | Age at | | | | |
|---|---|---|---|---|---|
| | challenge | Months | | | |
| | Yr | since | tTG- | DGP- | |
| Subject | (mths) | diagnosis | IgA | IgG | T cell response^{#} |
| C1 | 3 (38) | 16 | 1.2^{a} | 0^{a} | + |
| C2 | 5 (60) | 19 | 1.8^{a} | 1.2^{a} | ++ |
| C3 | 5 (53) | 13 | 5.7^{a} | 4.9^{a} | ++ |
| C4 | 5 (56) | 17 | ND | ND | + |
| C5 | 4 (49) | 5 | 10^{a} | 2^{a} | NR |
| C6 | 6 (71) | 9 | <5^{b} | <20^{b} | + |
| C7 | 5 (63) | 15 | <5^{b} | <20^{b} | + |
| C8 | 4 (50) | 10 | <5^{b} | <20^{b} | NR |
| C9 | 6 (70) | 11 | 2^{a} | 1.7^{a} | + |
| C10 | 5 (64) | 8 | 5.2^{a} | 3.1^{a} | NR |
| C11 | 6 (72) | 5 | 11^{a} | 65^{a} | + |
| C12 | 6 (73) | 54 | <5^{b} | <20^{b} | + |
| C13 | 5 (68) | 9 | <5^{b} | <20^{b} | + |
| C14 | 6 (83) | 48 | 3.4^{a} | 0.9^{a} | +++ |
| C15 | 7 (80) | 15 | 9.9^{a} | 1.3^{a} | + |
| C16 | 7 (83) | 7 | 19^{a} | 3.9^{a} | ++ |
| C17 | 8 (94) | 39 | 2.4^{a} | 6.2^{a} | NR |
| C18 | 9 (105) | 48 | 1.6^{a} | 0.1^{a} | +Gliadin only |
| C19 | 10 (113) | 34 | <5^{b} | <20^{b} | + |
| C20 | 10 (117) | 92 | 0.8^{a} | 13^{a} | ++ |
| C21 | 10 (115) | 24 | 21^{a} | 6.9^{a} | NR |
| C22 | 11 (131) | 57 | 2.4^{a} | 0.5^{a} | +++ |
| C23 | 11 (128) | 7 | 9.1^{a} | 10^{a} | ++ |
| C24 | 11 (126) | 39 | 0.9^{a} | 3.9^{a} | + |
| C25 | 11 (125) | 67 | <5^{b} | <20^{b} | + |
| C26 | 11 (137) | 102 | 0.3^{a} | 0.2^{a} | NR |
| C27 | 11 (135) | 19 | ND | ND | +++ |
| C28 | 12 (141) | 12 | 5.5^{c} | ND | + |
| C29 | 14 (162) | 36 | <5^{b} | <20^{b} | NR |
| C30 | 14 (164) | 119 | 1.8^{a} | 2^{a} | + |
| C31 | 14 (163) | 44 | <5^{b} | <20^{b} | NR |
| C32 | 15 (179) | 41 | <5^{b} | <20^{b} | NR |
| C33 | 15 (173) | 57 | ND | ND | + |
| C34 | 16 (187) | 54 | 4.9^{c} | 2^{b} | +++ |
| C35 | 17 (199) | 81 | 4.7^{c} | 2^{b} | + |
| C36 | 16 (195) | 20 | 11^{a} | 27^{a} | ++ |
| C37 | 17 (208) | 32 | 3.7^{c} | 13^{b} | +Gliadin/gluten |
| | | | | | only |
| C38 | 17 (208) | 29 | 17.6^{c} | 2^{b} | + |
| C39 | 17 (206) | 19 | 16.9^{c} | 4^{b} | +++ |
| C40 | 17 (210) | 87 | 4.8^{c} | 2^{b} | +++ |
| C41 | 17 (207) | 54 | 21.2^{c} | 2^{b} | +++ |
| A1 | 56 (674) | 461 | 15.9^{c} | 3^{b} | +++ |
| A2 | 58 (692) | 450 | 2^{c} | 1^{b} | + |
| A3 | 62 (745) | 531 | 5^{c} | 4^{b} | +++ |
| A4 | 70 (850) | 633 | 2^{c} | 2^{b} | +++ |

| Sub- | Symptoms (Mild +, Moderate ++, Severe +++) | | | | | | | | | | |
|---|---|---|---|---|---|---|---|---|---|---|---|
| ject | N | B | V | D | C | P | L | F | I | O | A |
| C1 | | | +++ | | | | | | | | |
| C2 | | | | | | | | | | | + |
| C3 | | | | | | | | | | | + |
| C4 | +++ | + | +++ | | | | | | | | |
| C5 | | | | | | ++ | + | | + | | |
| C6 | | + | | | | | | | | | |
| C7 | | | | | | | | | | | + |
| C8 | | | | | | ++ | | | +++ | +^{d} | |
| C9 | | + | | | | | + | | + | | |
| C10 | | | | | + | + | | | | | |
| C11 | | | | | | | | | | | + |
| C12 | + | | +++ | | | | | + | | +^{e} | |
| C13 | | | | + | | + | | | | | |
| C14 | | | | | | | | | | | + |
| C15 | | | | | | + | | | | | |
| C16 | | | | | | | | | + | | |
| C17 | | | | | | ++ | | | | | |
| C18 | | | | | | | | | | | + |
| C19 | | | | | | | | | | | + |
| C20 | | | | | + | + | | | | +^{f} | |
| C21 | | | | | | | | | | | + |
| C22 | | | +++ | | | | +++ | | | | |
| C23 | | | | | | | + | | | | |
| C24 | | | | | | | | | | | + |
| C25 | + | | | | | ++ | | | | | |
| C26 | | | | | | ++ | | | | | |
| C27 | + | | | | | + | ++ | | | | |
| C28 | | | | | | + | | | | | |
| C29 | | | | | + | | | | | | |
| C30 | | | | | | + | | | | | |
| C31 | + | | | +++ | | | ++ | | | | |
| C32 | +++ | + | | | + | ++ | | | | | |
| C33 | | | | | | | | | | | + |
| C34 | | | | | | + | + | | | +^{g} | |
| C35 | | | | | | | | | | | + |
| C36 | +++ | | + | | | | +++ | + | | | |
| C37 | ++ | | | | | ++ | + | | | | |
| C38 | | | | | | | | + | | | |
| C39 | | | | | | + | ++ | | | | |
| C40 | | | +++ | | | | | | | | |
| C41 | | | | | | | | | | | + |
| A1 | +++ | | | | | | | | | | |
| A2 | +++ | +++ | +++ | | | ++ | ++ | ++ | | | |
| A3 | +++ | + | +++ | | | | + | + | | | |
| A4 | + | | | | | | | | | | |

tTG (tissue transglutaminase) and DGP (deamidated gliadin peptide) measured prior to wheat challenge.
Normal serology range: ^{a}tTG-IgA (0-6) and DGP-IgG (0-6); ^{b}tTG-IgA (<5) and DGP-IgG (<20); ^{c}tTG-IgA (<20). ^{#}T cell response: <50 SFU/10⁶ PBMC +, 50-100 SFU/10⁶ PBMC ++, >100 SFU/10⁶ PBMC +++.
Symptoms: N = nausea, B = bloating, V = vomiting, D = diarrhoea, C = constipation, P = abdominal pain/cramping, L = lethargy, F = flatulence, I = irritable/moody, A = asymptomatic, O = other (^{d}rash, ^{e}poor appetite, ^{f}headache, ^{g}mouth ulcers).
ND (not done),
NR (non-responder).

Gluten-Specific T Cells are Induced by Wheat Challenge in Children with CD and Gluten Peptide Specificity and Dominance is Comparable to Adults

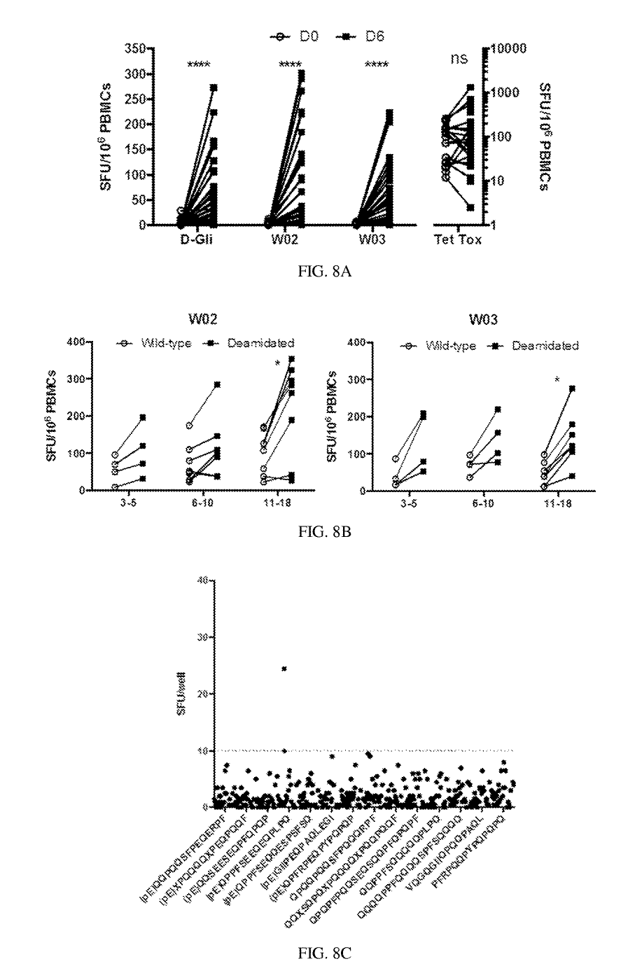

After oral wheat challenge, significant IFN-γ ELISpot responses were detectable in most CD volunteers (30/41; 73%) to at least one wheat gluten peptide (Table 8; Peptide details in Table 7). Responses to whole protein (gliadin and/or gluten) were less consistent, present in 23/30 of wheat gluten peptide responders and in 2 participants who did not respond to any wheat gluten peptides. Each age group contained a similar proportion of responders 3-5 yrs: 10/13, 76.9%; 6-10 yrs: 10/12, 83.3%; 11-18 yrs: 12/16, 75% (p=NS, Chi-Square test). Responses were seen on day 6 (D6) following oral wheat challenge, but not prior on D0except in one volunteer who also had a low-positive response to deamidated gliadin on D0 (FIG. 8A; n=28; deamidated gliadin, W02 (LQPFPQPELPYPQPQ, SEQ ID NO: 7) containing the wheat α-gliadin T cell epitopes DQ2.5-glia-α1a and α2, and W03 (QPFPQPEQPFPWQ, SEQ ID NO: 50) containing the wheat w-gliadin T cell epitopes DQ2.5-glia-ω1 and ω2 are shown). Tetanus toxoid responses did not differ between D0 and D6. The presence of a T cell response to gluten or gluten peptide was not affected by positive CD serology at baseline or DQ2.5 zygosity status (p=NS for both, Fisher's exact test).

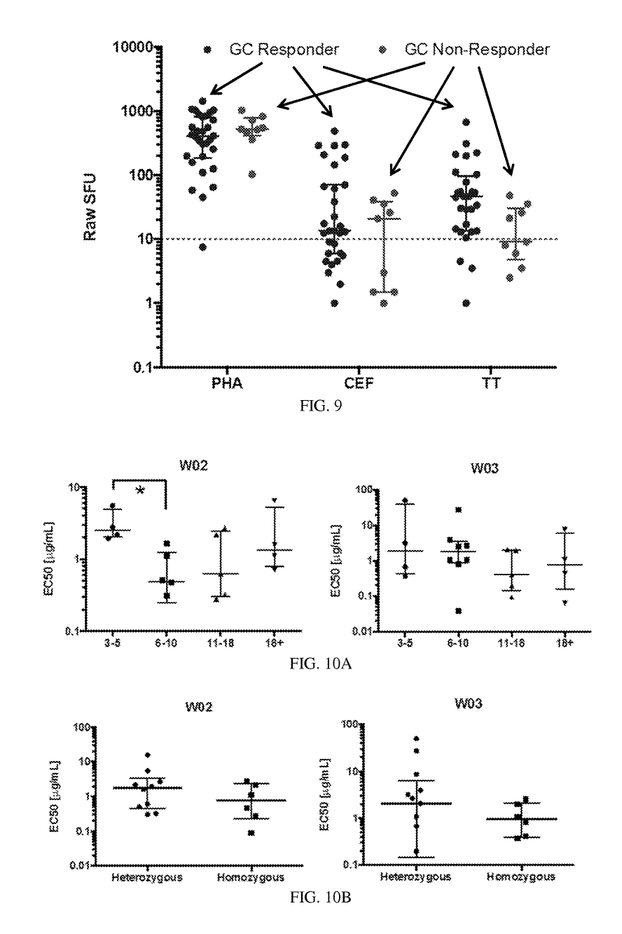

There was a clear preference for deamidated antigens compared to their native counterparts (FIG. 8B W02 and W03; and Table 8). The highest IFN-γ ELISpot responses were commonly noted to a gluten peptide compared to whole protein antigen (gliadin or gluten) in most cases (22/32; 69%, including two protein only responders), suggesting these peptides were dominant and responsible for most of the immune response to whole protein. Overall, volunteers responded to a number of wheat gluten-derived peptides (Table 8). Responses >70% of the maximal peptide response were seen in the majority of individuals against peptides containing the immunodominant DQ2.5-glia-α1/2 ω1/2 epitopes. Dominant responses to other peptide sequences were also occasionally seen in a small number of individuals. Non-response to gluten, gliadin and any gluten peptide was significantly associated with a poor response to CEF, tetanus toxoid and PHA (FIG. 9; P <0.005, Chi-square test). Following wheat challenge, in vivo polyclonal T cell responses against the novel immunogenic gluten peptides described by Vader et al. (20) were poor and seen in only 2/30 (6.7%) volunteers to the sequence QPPFSEEQEQPLPQ (SEQ ID NO: 172) (FIG. 8C).

Immunogenic peptides were ranked by magnitude of response to establish the hierarchy of gluten peptides (Table 8). Furthermore, “dominance scores” within each age grouping and overall were calculated (Table 14). 12/70 wheat gluten peptides were associated with dominance scores greater or equal to 30 for all age groups. Notably, the most dominant four peptides across all age groups corresponded closely to those observed in adults following wheat challenge (14) and included: W02 (LQPFPQPELPYPQPQ, SEQ ID NO: 7) and W01 (LPYPQPELPYPQP, SEQ ID NO: 64) both containing the DQ2.5-glia-α1b and α2 epitopes, W06 (LQPFPQPELPFPQP, SEQ ID NO: 70) containing a homolog of DQ2.5-glia-α1a, and W03 (QPFPQPEQPFPWQP, SEQ ID NO: 8) containing the DQ2.5-glia-ω1/ω2 epitopes. The dominance hierarchy also contained the native versions of W01, W02, and W04 (QPFPQPQQPIPVQ, SEQ ID NO: 158) but their deamidated equivalents were ranked higher in all cases (Table 14). An additional three peptides had dominance scores greater than 30 in the 3-5 and 6-10 yr olds and these contained sequences homologous to W03.

TABLE 14
Dominance scores for wheat-derived peptides in children with celiac disease after
wheat challenge.

| | | | | Dominance scores | | | | |
|---|---|---|---|---|---|---|---|---|
| | Peptide | | Defined/ | | | | | Mean |
| Peptide | sequence (core | Gliadin | predicted T | 3-5 | 6-10 | 11-18 | 18+ | all |
| name | in bold) | source | cell epitopes | yrs | yrs | yrs | yrs^{#} | ages |
| W02 | LQPFPQPELPYPQP | α | DQ2.5-glia- | 77 | 57 | 60 | 58 | 64 |
| | Q (SEQ ID NO: | | α1a/α2 | | | | | |
| | 7) | | | | | | | |
| W01 | LPYPQPELPYPQP | α | DQ2.5-glia- | 65 | 72 | 51 | 69 | 62 |
| | (SEQ ID NO: | | α1b/α2 | | | | | |
| | 64) | | | | | | | |
| W06 | LQPFPQPELPFPQP | α | PFPQPELPF; | 58 | 53 | 54 | 15 | 54 |
| | (SEQ ID NO: | | PQPELPFPQ | | | | | |
| | 70) | | | | | | | |
| W03 | QPFPQPEQPFPWQP | ω | DQ2.5-glia- | 65 | 51 | 46 | 35 | 54 |
| | (SEQ ID NO: 8) | | ω1/ω2 | | | | | |
| W26 | PFPLQPEQPFPQP | ω | LQPEQPFPQ | 46 | 62 | 35 | 0 | 47 |
| | (SEQ ID NO: | | | | | | | |
| | 109) | | | | | | | |
| W08 | LQPFPQPELPYSQP | α | DQ2.5-glia-α1a | 45 | 52 | 40 | 8 | 45 |
| | (SEQ ID NO: | | | | | | | |
| | 79) | | | | | | | |
| W04 | QPFPQPEQPIPVQ | ω | PFPQPEQPI; | 43 | 44 | 48 | 31 | 45 |
| | (SEQ ID NO: | | PQPEQPIPV | | | | | |
| | 152) | | | | | | | |
| W01 | LPYPQPQLPYPQP | α | DQ2.5-glia- | 31 | 49 | 35 | NT | 39 |
| (WT) | (SEQ ID NO: | | α1b/α2 | | | | | |
| | 67) | | | | | | | |
| W02 | LQPFPQPQLPYPQP | α | DQ2.5-glia- | 30 | 43 | 35 | NT | 37 |
| (WT) | (SEQ ID NO: | | α1a/α2 | | | | | |
| | 85) | | | | | | | |
| W04 | QPFPQPQQPIPVQ | ω | PFPQPQQPI; | 33 | 43 | 31 | NT | 36 |
| (WT) | (SEQ ID NO: | | PQPQQPIPV | | | | | |
| | 158) | | | | | | | |
| W16 | FPQPEQEFPQPQQ | γ | PQPEQEFPQ | 23 | 47 | 26 | 0 | 33 |
| | (SEQ ID NO: | | | | | | | |
| | 31) | | | | | | | |
| W13 | LQPFPQPELPYLQP | α | DQ2.5-glia-α1a | 34 | 32 | 30 | 4 | 32 |
| | (SEQ ID NO: | | | | | | | |
| | 73) | | | | | | | |
| W32 | PFPEQPEQPFPQP | γ | DQ2.5-glia-γ4c | 26 | 34 | 28 | 0 | 30 |
| | (SEQ ID NO: | | | | | | | |
| | 104) | | | | | | | |
| W05 | QPFPQPEQPFSQQ | γ | DQ2.5-glia-ω1; | 23 | 33 | 27 | 15 | 28 |
| | (SEQ ID NO: | | PQPEQPFSQ | | | | | |
| | 151) | | | | | | | |
| W07 | QPFPQPEQPFCQQ | γ | DQ2.5-glia-ω1; | 40 | 20 | 16 | 8 | 24 |
| | (SEQ ID NO: | | DQ2.5-glia-γ4d | | | | | |
| | 149) | | | | | | | |

Dominance scores were calculated for each peptide as the mean score for each age group and the mean of all subjects (percentage of maximal peptide response from Table 8). Top 15 peptides dominant in one subgroup are shown. # Corresponding adult data shown for comparison (14). NT=not tested.

### HLA DQ2.5 Zygosity Status but Not Age or Time Since Diagnosis Influence the Magnitude of the Gluten-Specific T Cell Response

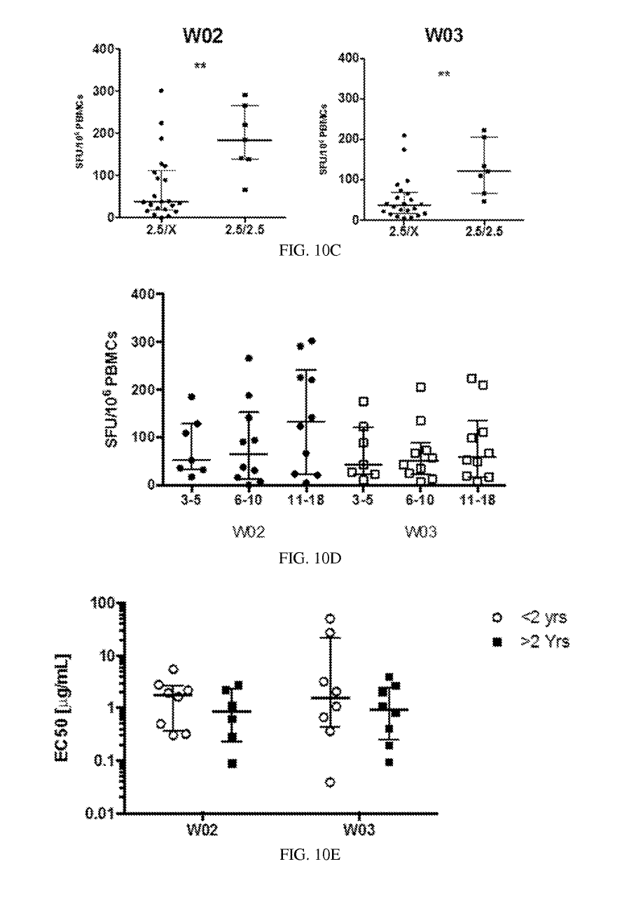

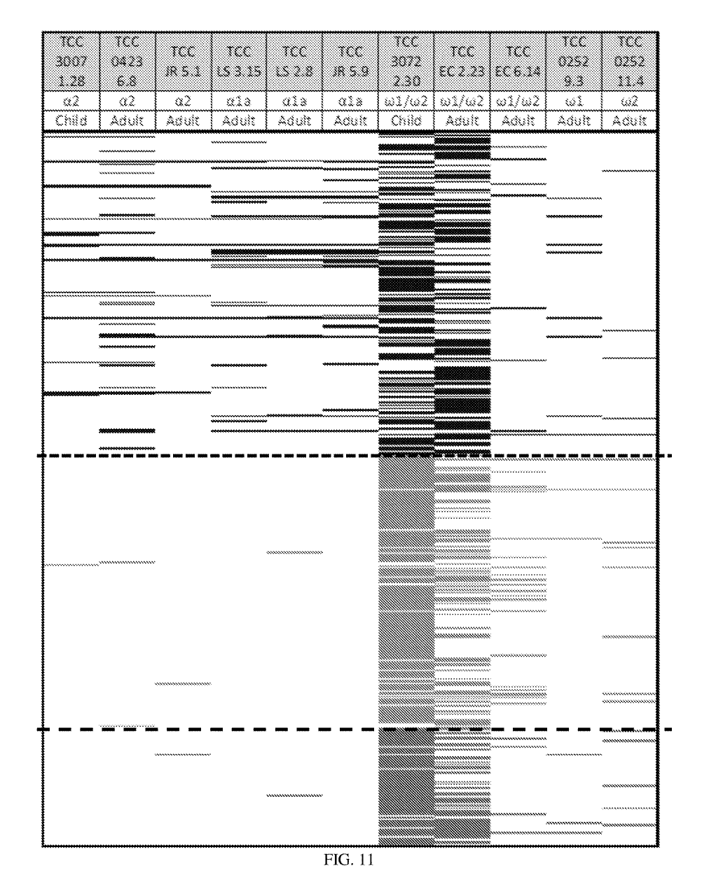

Dose-response curves and half maximal effective concentration (EC50) values were calculated in 17 children (n=4 3-5 yrs; n=8 6-10 yrs, and n=5 11-18 yrs) and four adults (18-70 yrs) based on T cell responses to W02 and W03 (FIGS. 11A-E). Median EC50 were similar across each age group for W03 (FIG. 10A). EC50 values for W02 were similar between 6-10 yrs and 11-18 yrs and adults, but a difference was observed between 3-5 yrs and 6-10 yrs (FIG. 10A). EC50 values were not statistically different between children who were heterozygous or homozygous for HLA-DQ2.5 (FIG. 10B), but a trend towards a lower EC50 in homozygotes was noted. Notably, the overall magnitude of T cell responses to W02 and W03 was significantly greater for volunteers who were HLA-DQ2.5 homozygotes compared to heterozygotes (FIG. 10C). This difference was not seen when comparing age groups (FIG. 10D; p=NS, Kruskal-Wallis).

EC50 values were also compared based on the years since diagnosis of CD, as this may be a separate factor to volunteer age that could impact on the consistency and hierarchy of T cell responses. As expected, the elapsed time from diagnosis was lowest in the youngest (3-5 yr) age group (3-5 yrs 0.4-4.4yrs; median 1 yr, 6-10 yrs 0.6-7.7 yrs; median 3.3 yrs, and 11-18 yrs 1-9.9yrs; median 3.6 yrs; p<0.05, Kruskal Wallis). However the mean EC50 to either W02 or W03 was not different between less than 2 years or greater than 2 years from the time of diagnosis (FIG. 10E).

Both Polyclonal and Clonal Gluten-Specific T Cells Cross-React with Hordein and Secalin Peptides

It was sought to determine the level of barley and rye grain cross-reactivity in T cells from CD children raised in vivo and in vitro. It was found that polyclonal T cells induced by oral wheat challenge cross-reacted to a series of peptides derived from barley hordein and rye secalin (Table 10; n=22). Fifteen of twenty-two (68%) CD children responded to W03 containing the same T cell epitope sequence in wheat (DQ2.5-glia-ω1/ω2) and barley (DQ2.5-hor-1) (Table 10), and in most cases, also responded to other peptides containing homologous sequences within hordein and secalin.

A TCC specific to DQ2.5-glia-α2 and one specific to DQ2.5-glia-ω1/ω2 were raised from two different children with CD and their recognition of comprehensive wheat gliadin, barley hordein, and rye secalin peptide libraries was assessed (FIG. 11). Previously isolated TCC from adults with CD (14) were tested against the same libraries for comparison. TCC 3007.28 (specific to DQ2.5-glia-α2) showed minimal reactivity to hordein or secalin peptides, consistent with the observation that peptides containing the immunodominant wheat T cell epitopes DQ2.5-glia-α1a and DQ2.5-glia-α2 are infrequent in barley or rye (FIG. 11; positive response to 575 peptides; peptide sequences not shown). In contrast, TCC specific to DQ2.5-glia-ω1/2 showed substantially more immunoreactivity to a range of hordein and secalin peptides that encompass both epitopes. Notably, the cross-reactivity patterns of the TCC from children for secalin and hordein were very similar to those raised from adults (14), and showed the same bias of high cross-reactivity for TCC specific for DQ2.5-glia-ω1/2 and more restricted cross-reactivity for TCC specific for specific for DQ2.5-glia-α1a/α2 (FIG. 11).

### Discussion

The assessment of polyclonal T cells induced by short-term wheat challenge indicates remarkable consistency in T cell recognition of immunodominant wheat gluten epitopes, specifically DQ2.5-glia-α1/α2 and DQ2.5-glia-ω1/ω2, in HLA DQ2.5+ children comparable to adults with CD from England, Italy, Norway, and Australia (11, 14, 15, 24). While this is not an unbiased, comprehensive study of gluten T cell epitopes in pediatric CD, the finding that T cell responses to select gliadin peptides were generally greater than to whole gluten antigen suggests the contribution of other untested peptides to the total gluten immune response is minimal. As it is difficult to accurately determine true disease duration because of variability in the time to establish a diagnosis, T cell responses were arbitrarily compared in those who undertook wheat challenge less than two years since diagnosis to those participating more than two years from diagnosis, and observed no differences. As the T cell epitope dominance hierarchy was not affected by age or the time from diagnosis, these results suggest that the recall T cell response to specific gluten peptides develops early in disease pathogenesis, is well established when CD is eventually diagnosed, and remains consistent over time.

These findings highlight the dominance of DQ2.5-glia-α1/α2 and DQ2.5-glia-ω1/ω2 after wheat ingestion in children with CD, and confirm the importance of deamidation in enhancing bioactivity of most immunogenic peptides. These findings challenge in vitro data that indicate a lower rate of response to dominant T cell epitopes, a lower rate of dependence on deamidation for bioactivity, and implicate a series of novel immunogenic peptides. Vader et al. showed that only 8/16 TCL from 25 Dutch children with CD responded to DQ2.5-glia-α1a/α2 regarded as immunodominant in adults with CD (20). In the same study only 2/4 TCL isolated from adults with CD responded to these α-gliadin epitopes, contrasting with 17/17 (100%) TCLs isolated from adult Norwegian CD donors (10), suggesting that methodological differences are a contributory factor underlying these differences. Using in vitro culture of PBMC from newly diagnosed children with CD, Liu et al. detected responses to a peptide containing a deamidation variant of DQ8-glia-α1 in 60% of CD children, including some not even carrying HLA-DQ8; responses to DQ2.5-glia-α1a and α2 were seen in 3/10 and 0/10 individual CD children, respectively (25). Lammi et al. detected PBMC responses to tTG-treated gliadin in 11/20 untreated CD children but no proliferation in 15 CD children to peptides containing DQ2.5-glia-α1a/1b/2 (26). All of these in vitro studies are prone to artefact due to prolonged in vitro culture and the use of potent mitogens which may expand naöve T cell populations and affect the composition and function of the T cell population of interest (24). Vader et al. postulated that epitope spreading accounts for the greater heterogeneity in gluten peptide responses in children and the immune response focuses over time on immunodominant deamidated epitopes due to greater binding affinity. However, epitope spreading typically occurs within weeks, not years, of an immune response (27) and this is supported by the findings herein that suggest the repertoire of gluten-specific T cells is well established by the time CD is diagnosed. A potential role for deamidated epitopes has been highlighted in a humanized HLA-DQ8 transgenic mouse model (28). Immunization with a native gluten peptide comprising the commonly recognized DQ8-glia-α1 epitope (QGSFQPSQQ, SEQ ID NO: 173) can recruit a T cell population that is not only largely cross-reactive but also substantially heteroclitic against the corresponding deamidated peptides; furthermore, the frequency of T cells recognizing the deamidated peptide was consistently highly increased after immunization with a mixture of both native and deamidated peptides. It is feasible that in the early phase of disease pathogenesis T cells are recruited by native peptide and then continuously activated once inflammation is triggered, which enhances cytoplasmic tTG release and leads to the generation of deamidated peptide.

It has been reported that the HLA-DQ2.5 gene dose has a strong quantitative effect on the magnitude of gluten-specific T-cell responses (29). Although the overall magnitude of T cell responses to dominant peptides were higher in HLA-DQ2.5 homozygotes the EC50s were not significantly lower. Responses to subdominant peptides in gliadin, hordein, and secalin appeared broader in homozygotes, possibly due to more efficient presentation of these peptides on the surface of antigen presenting cells (APCs), or to more efficient priming/expansion of cognate T cells in HLA-DQ2.5 homozygous CD. HLA-DQ2.5 homozygosity, specifically, two copies of the HLA-DQB1*02 allele, increases the risk of developing CD (7, 25), and in some reports is associated with a more severe clinical phenotype characterized by younger age at diagnosis, more severe symptoms and slower recovery after commencing a GFD (30). The study herein did not identify an association between homozygosity and earlier disease onset or more severe histology at diagnosis, although the sample of HLA-DQ2.5 homozygous volunteers was relatively small.

High redundancy of gluten peptide recognition by T cells specific for dominant peptides is a key feature underpinning the feasibility of peptide-based applications in CD (14). In adults with CD, the study herein has shown the polyclonal T cell response induced after gluten challenge specific for three peptides from wheat (W02 and W03) and barley (B08; EPEQPIPEQPQPYPQQ, SEQ ID NO: 12) that each encompass the immunodominant T cell epitopes DQ2.5-glia-α1a/α2, DQ2.5-glia-ω1/ω2 and DQ2.5-hor-3 respectively, were equivalent to as much as 90% of that elicited by optimal concentrations of tTG-treated wheat gliadin, barley hordein, or the most immunogenic secalin fraction (w-secalin). Moreover, TCCs isolated from adults with CD specific for DQ2.5-glia-α1a/α2, DQ2.5-glia-ω1/ω2, DQ2.5-hor-3 and a rye epitope (DQ2.5-sec-1) recognise almost 90% of the T cell stimulatory gluten peptides from all of the toxic cereals in CD. Notably, TCCs specific for DQ2.5-glia-ω1/ω2 were the most cross-reactive, while TCCs specific for DQ2.5-glia-α1a/α2, generally regarded the most “important” immunodominant T cell epitopes driving CD pathogenesis, showed the least amount of cross-reactivity. In this study it was shown that TCC specific for the immunodominant wheat T cell epitopes from children with CD shared the same cross-reactivity patterns as TCCs from adults with CD. The findings support the feasibility of peptide-based applications using a discrete number of dominant peptides in children with CD.

These findings indicate that the specificity of the gluten-specific T cell response reactivated by oral wheat challenge in children with CD does not differ from adults. Stability of epitopes recognized by gluten-reactive CD4+ T-cells after diagnosis of CD whether in childhood or adult life indicates clinical applications of T-cell epitopes should be relevant to patients of all ages.

### Material and Methods

### Subjects and Oral Grain Challenge

All volunteers had biopsy-proven CD diagnosed according to ESPGHAN criteria (34), and possessed both alleles (HLA-DQA1*05 and HLA-DQB1*02 encoding the major CD-determining HLA-DQ haplotype (HLA-DQ2.5+) but did not possess either HLA-DQ allele encoding HLA-DQ8. Volunteers were required to have followed a strict gluten-free diet for at least the previous three months. The Australian cohort consisted of 41 pediatric CD volunteers (aged 3-17; median 9 yrs; 17 M:24 F) split into three groups: 3-5, 6-10, and 11-18. An additional four adults (18+) with CD were recruited for comparison of T cells responses (See Table 1).

Short-term oral wheat challenge was performed as previously described for adults with CD (14), however the amount of bread consumed daily was modified for the younger age: 3-5 yr 1 slice of bread, 6-10 yr 2 slices, and 11-18 yr 3 slices. This corresponded to a similar amount of daily gluten intake across all age groups (˜0.2 g/kg gluten based on median weight using weight-for-age percentile charts: http://www.cdc.gov/growthcharts/clinical_charts.htm). Blood for T cell studies was collected by pediatric phlebotomists in lithium heparin vacutainers before and six days after commencing the oral challenge. Venesection volume was determined by weight based on the WHO guideline (35). Volunteers completed symptom diaries where symptom type and severity (mild, moderate, or severe) were reported daily for the six days following gluten challenge.

### Antigens

To optimise assessment of peptides with a limited blood volume from pediatric donors, a modified library containing both wild-type and in silico deamidated versions of the most immunogenic wheat gliadin and glutenin peptide sequences described previously (14) was assessed (Table 7). When collected blood volume allowed, a series of deamidated peptides known to be immunogenic in a large group of adults with CD in vivo were also assessed in the Australian CD cohort: barley hordein (n=22), rye secalin (n=30), and oats avenin (n=2; one wild-type) (14, 36). The screening library was custom synthesized and the identity of each peptide was confirmed by LC-MS (GL Biochem, Minhang, China). Additional high quality (>80%) peptides were synthesised by Pepscan (Lelystad, Netherlands) or GL Biochem. Comprehensive gliadin (n=1535), hordein (n=1444), and secalin (n=350) peptide libraries consisting of wildtype and in silico deamidated sequences (14) were used to screen TCC to determine redundancy of peptide recognition. Whole gluten was assessed in addition to gliadin (#101778; ICN Biomedicals, OH, USA) to determine if untested glutenin peptides contributed substantially to the whole gluten response. Gluten and gliadin were incubated in 10-fold excess with chymotrypsin (Sigma #C3142) in ammonium bicarbonate (pH 8) for 4 h at 37° C. and was then boiled for 15 min. Protein concentration of the hydrolysate was determined using the BioRad Protein Assay Dye Reagent #500-0006 method (BioRad, CA, USA). Deamidation with guinea pig liver tTG (Sigma T5398) was as described previously (11, 13).

### IFN-γ ELISpot Assay

Peripheral blood mononuclear cells (PBMC) were isolated from whole blood using Ficoll-Paque™ Plus density-gradient centrifugation (GE Healthcare). IFN-γ ELISpot (Mabtech) assays were performed and analyzed as previously described (14). Briefly, PBMC were incubated overnight with individual peptides (50 μg/ml), with medium alone as negative control, and with one or more positive controls including Tetanus toxoid (TT; CSL, Australia), phytohemagglutinin-L (PHA-L; Sigma USA), or CEF cocktail (Mabtech). Spot forming units (SFU) in individual wells were counted using an automated ELISPOT reader (AID ELISpot Reader System, Autoimmun Diagnostika GmbH; Strassberg, Germany or in Italy on a AELVIS ELISpot reader, Hannover, Germany). Wells showing more than 10 SFU and >3× the SFU counted in wells containing PBMC incubated with medium alone were regarded as positive. Dominance scores for each peptide were defined using the IFN-γ response elicited as a proportion of the most active peptide screened, and then averaged across each volunteer group. SFU were adjusted to one million PBMC plated to enable comparisons. EC50 values, representing the half maximal peptide concentration, were calculated using Prism 6.0 software on a dose curve containing 8 peptide concentrations ranging from 0.1-50 μg/mL.

### T Cell Cloning

TCC were generated as previously described (14). Briefly, CFSE-labeled PBMC were incubated with antigen for 7 days in IMDM complete media supplemented with 5% heat-inactivated pooled human serum (PHS), 2 mM GlutaMAX™, 100 μM MEM non-essential amino acids (both from Gibco, Invitrogen), and 50 μM 2-mercaptoethanol (Sigma). Proliferating cells were sorted with one cell per well in 96-well plates and incubated in the presence of IL-2, IL-4, anti-CD3 mAb, irradiated allogeneic PBMC and JY-EBV (an Epstein-Barr virus-immortalised B cell line). TCC were expanded and maintained in IL-2 and IL-4 and tested for specificity by ELISpot as described above with minor modifications. TCC (1000-2000/well) were incubated with relevant peptide (25 μg/ml unless otherwise stated) and irradiated HLA-matched PBMC or HLA-DQ2.5-expressing T2 cells as antigen-presenting cells (25,000-50,000/well). Epitopes recognized by TCC were tested for HLA-DQ2.5 restriction using an anti-human blocking antibody specific for HLA-DQ and the HLA-DQ2.5-expressing T2 cells, TCR Vbeta usage by the IOTest Beta Mark TCR V kit (Beckman Coulter), and with lysine scans to work out minimal epitopes (39).

### IFN-γ Secretion Assay

Either fresh or cryopreserved PBMC from known T cell responders were rested overnight in IMDM complete media. CD4+ T cells were enriched using the EasySep Negative Selection Human CD4+ T cell enrichment kit (Stem Cell Technologies), following manufacturer's recommendations. CD4+ T cells were stimulated with or without 50 μg/ml peptide, in addition to 10 μg/ml purified anti-CD28 antibody and 1.25 μg/ml anti-CD49d antibody (both from Biolegend), and autologous PBMC at a 1:1 ratio, in 96-well round bottom plates in replicate wells containing a final volume of 150 μl IMDM complete media containing 20 μg/ml DNase I (Roche). The MACS IFN-γ secretion assay—Detection kit (FITC) human (Miltenyi Biotec) was used to enable cell sorting of IFN-γ secreting, gluten-specific CD4+ T cells, following manufacturer's recommendations. In addition to IFN-γ-FITC, cells were co-stained with CD4-APC and CD14-PerCP (BD Biosciences), CD69-PECy7 (Biolegend), and propidium iodide (Sigma). Gluten-specific (IFN-γ+CD69+CD4+) cells were single-cell sorted into 96-well PCR plates (Eppendorf) up to 80 wells and one column left as non-template controls, on a BD FACS Aria. Wells were capped with strip lids, and stored frozen for later processing.

### REFERENCES

1. R. P. Anderson, and B. Jabri, Vaccine against autoimmune disease: antigen-specific immunotherapy. Curr Opin Immuno1:1-8. (2013).

2. L. M. Sollid, S. W. Qiao, R. P. Anderson, C. Gianfrani, and F. Koning, Nomenclature and listing of celiac disease relevant gluten T-cell epitopes restricted by HLA-DQ molecules. Immunogenetics 64:455-460. (2012).

3. S. Simell, A. Kupila, S. Hoppu, A. Hekkala, T. Simell, M. R. Stahlberg, M. Viander, T. Hurme, M. Knip, J. Ilonen, et al., Natural history of transglutaminase autoantibodies and mucosal changes in children carrying HLA-conferred celiac disease susceptibility. Scand J Gastroenterol 40:1182-1191. (2005).

4. R. S. Choung, I. C. Ditah, A. M. Nadeau, A. Rubio-Tapia, E. V. Marietta, T. L. Brantner, M. J. Camilleri, S. V. Rajkumar, O. Landgren, J. E. Everhart, et al., Trends and racial/ethnic disparities in gluten-sensitive problems in the United States: findings from the national health and nutrition examination surveys from 1988 to 2012. Am J Gastroenterol 110:455-461. (2015).

5. A. Rubio-Tapia, R. A. Kyle, E. L. Kaplan, D. R. Johnson, W. Page, F. Erdtmann, T. L. Brantner, W. R. Kim, T. K. Phelps, B. D. Lahr, et al., Increased prevalence and mortality in undiagnosed celiac disease. Gastroenterology 137:88-93. (2009).

6. S. L. Vriezinga, R. Auricchio, E. Bravi, G. Castillejo, A. Chmielewska, P. Crespo Escobar, S. Kolacek, S. Koletzko, I. R. Korponay-Szabo, E. Mummert, et al., Randomized feeding intervention in infants at high risk for celiac disease. N Engl J Med 371:1304-1315. (2014).

7. E. Lionetti, S. Castellaneta, R. Francavilla, A. Pulvirenti, E. Tonutti, S. Amarri, M. Barbato, C. Barbera, G. Barera, A. Bellantoni, et al., Introduction of gluten, HLA status, and the risk of celiac disease in children. N Engl J Med 371:1295-1303. (2014).

8. S. Husby, S. Koletzko, I. R. Korponay-Szabo, M. L. Mearin, A. Phillips, R. Shamir, R. Troncone, K. Giersiepen, D. Branski, C. Catassi, et al., European Society for Pediatric Gastroenterology, Hepatology, and Nutrition guidelines for the diagnosis of coeliac disease. J Pediatr Gastroenterol Nutr 54:136-160. (2012).

9. K. Karell, A. S. Louka, S. J. Moodie, H. Ascher, F. Clot, L. Greco, P. J. Ciclitira, L. M. Sollid, J. Partanen, and D. European Genetics Cluster on Celiac, HLA types in celiac disease patients not carrying the DQA1*05-DQB1*02 (DQ2) heterodimer: results from the European Genetics Cluster on Celiac Disease. Human Immunology 64:469-477. (2003).

10. H. Arentz-Hansen, R. Korner, O. Molberg, H. Quarsten, W. Vader, Y. M. Kooy, K. E. Lundin, F. Koning, P. Roepstorff, L. M. Sollid, et al., The intestinal T cell response to alpha-gliadin in adult celiac disease is focused on a single deamidated glutamine targeted by tissue transglutaminase. Journal of Experimental Medicine 191:603-612. (2000).

11. R. P. Anderson, P. Degano, A. J. Godkin, D. P. Jewell, and A. V. Hill, In vivo antigen challenge in celiac disease identifies a single transglutaminase-modified peptide as the dominant A-gliadin T-cell epitope. Nature Medicine 6:337-342. (2000).

12. M. Bodd, M. Raki, E. Bergseng, J. Jahnsen, K. E. Lundin, and L. M. Sollid, Direct cloning and tetramer staining to measure the frequency of intestinal gluten-reactive T cells in celiac disease. Eur J Immunol 43:2605-2612. (2013).

13. R. P. Anderson, D. A. van Heel, J. A. Tye-Din, M. Barnardo, M. Salio, D. P. Jewell, and A. V. Hill, T cells in peripheral blood after gluten challenge in coeliac disease. Gut 54:1217-1223. (2005).

14. J. A. Tye-Din, J. A. Stewart, J. A. Dromey, T. Beissbarth, D. A. van Heel, A. Tatham, K. Henderson, S. I. Mannering, C. Gianfrani, D. P. Jewell, et al., Comprehensive, quantitative mapping of T cell epitopes in gluten in celiac disease. Sci Transl Med 2:41ra51. (2010).

15. A. Camarca, R. P. Anderson, G. Mamone, O. Fierro, A. Facchiano, S. Costantini, D. Zanzi, J. Sidney, S. Auricchio, A. Sette, et al., Intestinal T cell responses to gluten peptides are largely heterogeneous: implications for a peptide-based therapy in celiac disease. J Immunol 182:4158-4166. (2009).

16. W. Dieterich, E. Laag, H. Schopper, U. Volta, A. Ferguson, H. Gillett, E. O. Riecken, and D. Schuppan, Autoantibodies to tissue transglutaminase as predictors of celiac disease.[see comment]. Gastroenterology 115:1317-1321. (1998).

17. W. Dieterich, T. Ehnis, M. Bauer, P. Donner, U. Volta, E. O. Riecken, and D. Schuppan, Identification of tissue transglutaminase as the autoantigen of celiac disease.[see comment]. Nature Medicine 3:797-801. (1997).

18. A. A. Osman, T. Gunnel, A. Dietl, H. H. Uhlig, M. Amin, B. Fleckenstein, T. Richter, and T. Mothes, B cell epitopes of gliadin. Clinical & Experimental Immunology 121:248-254. (2000).

19. M. Aleanzi, A. M. Demonte, C. Esper, S. Garcilazo, and M. Waggener, Celiac disease: antibody recognition against native and selectively deamidated gliadin peptides. Clinical Chemistry 47:2023-2028. (2001).

20. W. Vader, Y. Kooy, P. Van Veelen, A. De Ru, D. Harris, W. Benckhuijsen, S. Pena, L. Mearin, J. W. Drijfhout, and F. Koning, The gluten response in children with celiac disease is directed toward multiple gliadin and glutenin peptides. Gastroenterology 122:1729-1737. (2002).

21. N. Ontiveros, J. A. Tye-Din, M. Y. Hardy, and R. P. Anderson, Ex-vivo whole blood secretion of interferon (IFN)-γ and IFN-γ-inducible protein-10 measured by enzyme-linked immunosorbent assay are as sensitive as IFN-γ enzyme-linked immunospot for the detection of gluten-reactive T cells in human leucocyte antigen (HLA)-DQ2.5+-associated coeliac disease. Clinical and Experimental Immunology 175:305-315. (2013).

22. S. W. Qiao, M. Raki, K. S. Gunnarsen, G. A. Loset, K. E. Lundin, I. Sandlie, and L. M. Sollid, Posttranslational modification of gluten shapes TCR usage in celiac disease. J Immunol 187:3064-3071. (2011).

23. A. Han, E. W. Newell, J. Glanville, N. Fernandez-Becker, C. Khosla, Y. H. Chien, and M. M. Davis, Dietary gluten triggers concomitant activation of CD4+ and CD8+ alphabeta T cells and gammadelta T cells in celiac disease. Proc Natl Acad Sci USA 110:13073-13078. (2013).

24. M. Raki, L. E. Fallang, M. Brottveit, E. Bergseng, H. Quarsten, K. E. Lundin, and L. M. Sollid, Tetramer visualization of gut-homing gluten-specific T cells in the peripheral blood of celiac disease patients. Proc Natl Acad Sci USA 104:2831-2836. (2007).

25. E. Liu, H. S. Lee, C. A. Aronsson, W. A. Hagopian, S. Koletzko, M. J. Rewers, G. S. Eisenbarth, P. J. Bingley, E. Bonifacio, V. Simell, et al., Risk of pediatric celiac disease according to HLA haplotype and country. N Engl J Med 371:42-49. (2014).

26. A. Lammi, P. Arikoski, O. Vaarala, T. Kinnunen, and J. Ilonen, Increased peripheral blood CD4+ T cell responses to deamidated but not to native gliadin in children with coeliac disease. Clin Exp Immunol 168:207-214. (2012).

27. C. J. Vanderlugt, and S. D. Miller, Epitope spreading. Curr Opin Immunol 8:831-836. (1996).

28. Z. Hovhannisyan, A. Weiss, A. Martin, M. Wiesner, S. Tollefsen, K. Yoshida, C. Ciszewski, S. A. Curran, J. A. Murray, C. S. David, et al., The role of HLA-DQ8 beta57 polymorphism in the anti-gluten T-cell response in coeliac disease. Nature 456:534-538. (2008).

29. W. Vader, D. Stepniak, Y. Kooy, L. Mearin, A. Thompson, J. J. van Rood, L. Spaenij, and F. Koning, The HLA-DQ2 gene dose effect in celiac disease is directly related to the magnitude and breadth of gluten-specific T cell responses. Proceedings of the National Academy of Sciences of the United States of America 100:12390-12395. (2003).

30. H. Karinen, P. Karkkainen, J. Pihlajamaki, E. Janatuinen, M. Heikkinen, R. Julkunen, V. M. Kosma, A. Naukkarinen, and M. Laakso, Gene dose effect of the DQB1*0201 allele contributes to severity of coeliac disease. Scand J Gastroenterol 41:191-199. (2006).

31. J. Petersen, V. Montserrat, J. R. Mujico, K. L. Loh, D. X. Beringer, M. van Lummel, A. Thompson, M. L. Mearin, J. Schweizer, Y. Kooy-Winkelaar, et al., T-cell receptor recognition of HLA-DQ2-gliadin complexes associated with celiac disease. Nat Struct Mol Biol 21:480-488. (2014).

32. S. W. Qiao, A. Christophersen, K. E. Lundin, and L. M. Sollid, Biased usage and preferred pairing of alpha- and beta-chains of TCRs specific for an immunodominant gluten epitope in coeliac disease. Int Immunol 26:13-19. (2014).

33. A. Christophersen, M. Raki, E. Bergseng, K. E. Lundin, J. Jahnsen, L. M. Sollid, and S. W. Qiao, Tetramer-visualized gluten-specific CD4+ T cells in blood as a potential diagnostic marker for coeliac disease without oral gluten challenge. United European Gastroenterol J 2:268-278. (2014).

34. J. Walker-Smith, S. Guandalini, J. Schmitz, D. Shmerling, and J. Visakorpi, Revised criteria for diagnosis of coeliac disease. Report of Working Group of European Society of Paediatric Gastroenterology and Nutrition. Archives of Disease in Childhood 65:909-911. (1990).

35. S. R. Howie, Blood sample volumes in child health research: review of safe limits. Bull World Health Organ 89:46-53. (2011).

36. M. Y. Hardy, J. A. Tye-Din, J. A. Stewart, F. Schmitz, N. L. Dudek, I. Hanchapola, A. W. Purcell, and R. P. Anderson, Ingestion of oats and barley in patients with celiac disease mobilizes cross-reactive T cells activated by avenin peptides and immuno-dominant hordein peptides. J Autoimmun 56:56-65. (2015).

37. A. Camarca, G. Radano, R. Di Mase, G. Terrone, F. Maurano, S. Auricchio, R. Troncone, L. Greco, and C. Gianfrani, Short wheat challenge is a reproducible in-vivo assay to detect immune response to gluten. Clin Exp Immunol 169:129-136. (2012).

38. A. Camarca, A. Del Mastro, and C. Gianfrani, Repertoire of gluten peptides active in celiac disease patients: perspectives for translational therapeutic applications. Endocr Metab Immune Disord Drug Targets 12:207-219. (2012).

39. S. I. Mannering, J. A. Dromey, J. S. Morris, D. J. Thearle, K. P. Jensen, and L. C. Harrison, An efficient method for cloning human autoantigen-specific T cells. J Immunol Methods 298:83-92. (2005).

40. G. C. Wang, P. Dash, J. A. McCullers, P. C. Doherty, and P. G. Thomas, T cell receptor alphabeta diversity inversely correlates with pathogen-specific antibody levels in human cytomegalovirus infection. Sci Transl Med 4:128ra142. (2012). 41. V. Venturi, K. Kedzierska, S. J. Turner, P. C. Doherty, and M. P. Davenport, Methods for comparing the diversity of samples of the T cell receptor repertoire. J Immunol Methods 321:182-195. (2007).

42. A. E. Magurran. 2003. Measuring Biological Diversity: Wiley-Blackwell. 264 pp.

### Equivalants

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

1. A method for treating Celiac disease in a child, the method comprising:
administering to a child having Celiac disease an effective amount of a composition comprising one or more peptides comprising an adult immunodominant epitope.

2. The method of claim 1, wherein the composition comprises at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 5), and EQPIPEQPQ (SEQ ID NO: 6).

3. The method of claim 1, wherein the composition comprises at least one of:
(i) a first peptide comprising the amino acid sequence PFPQPELPY (SEQ ID NO: 1) and PQPELPYPQ (SEQ ID NO: 2),
(ii) a second peptide comprising the amino acid sequence PFPQPEQPF (SEQ ID NO: 3) and PQPEQPFPW (SEQ ID NO: 4), and
(iii) a third peptide comprising the amino acid sequence PIPEQPQPY (SEQ ID NO: 5), or a third peptide comprising the amino acid sequence PIPEQPQPY (SEQ ID NO: 5) and EQPIPEQPQ (SEQ ID NO: 6);
optionally wherein the first, second, and/or third peptide are each independently 8-50 amino acids in length.

4-5. (canceled)

6. The method of claim 3, wherein (i) the first peptide comprises LQPFPQPQLPYPQPQ (SEQ ID NO: 7); the second peptide comprises QPFPQPQQPFPWQP (SEQ ID NO: 8); and the third peptide comprises PQQPIPQQPQPYPQQ (SEQ ID NO: 9), optionally wherein the first, second, and/or third peptide are each independently 15-30 amino acids in length.

7. (canceled)

8. The method of claim 3, wherein the first peptide comprises the amino acid sequence ELQPFPQPELPYPQPQ (SEQ ID NO:10), wherein the N-terminal glutamate is a pyroglutamate and the C-terminal glutamine is amidated; the second peptide comprises the amino acid sequence EQPFPQPEQPFPWQP (SEQ ID NO: 11), wherein the N-terminal glutamate is a pyroglutamate and the C-terminal proline is amidated; and the third peptide comprises the amino acid sequence EPEQPIPEQPQPYPQQ (SEQ ID NO: 12), wherein the N-terminal glutamate is a pyroglutamate and the C-terminal glutamine is amidated;
optionally wherein the amino acid sequence of the first peptide is ELQPFPQPELPYPQPQ (SEQ ID NO: 10), wherein the N-terminal glutamate is a pyroglutamate and the C-terminal glutamine is amidated; the amino acid sequence of the second peptide is EQPFPQPEQPFPWQP (SEQ ID NO: 11), wherein the N-terminal glutamate is a pyroglutamate and the C-terminal proline is amidated; and the amino acid sequence of the third peptide is EPEQPIPEQPQPYPQQ (SEQ ID NO: 12), wherein the N-terminal glutamate is a pyroglutamate and the C-terminal glutamine is amidated.

9. (canceled)

10. The method of claim 3, wherein the composition comprises the first and second peptide, the first and third peptide, or the second and third peptide, optionally wherein the composition comprises the first, second, and third peptide.

11-12. (canceled)

13. The method of claim 10, wherein the composition comprises
(i) 50 micrograms of the first peptide and an equimolar amount of each of the second and third peptides;
(ii) 26.5 nmol of each of the first, second, and third peptides;
(iii) 25 micrograms of the first peptide and an equimolar amount of each of the second and third peptides; or
(iv) 13.2 nmol of each of the first, second, and third peptides.

14-16. (canceled)

17. The method of claims 1, wherein
(i) the composition is administered intradermally;
(ii) the composition is administered as a bolus by intradermal injection;
(iii) the composition is formulated as a sterile, injectable solution;
(iv) the child is HLA-DQ2.5 positive;
(v) the child is on a gluten-free diet; and/or
(vi) the composition is administered twice a week for up to 8 weeks.

18-22. (canceled)

23. The method of claim 1, wherein the method further comprises:
(i) further administering to the child the composition comprising 50 micrograms of the first peptide and an equimolar amount of each of the second and third peptides, and/or
(ii) further administering to the child the composition comprising 26.5 nmol of each of the first, second, and third peptides,
optionally wherein the further administering is twice a week for up to 8 weeks.

24-25. (canceled)

26. A method for identifying a child as having or at risk of having Celiac disease, the method comprising:
determining a T cell response to a composition comprising one or more peptides comprising an adult immunodominant epitope in a sample comprising a T cell from the child; and assessing whether or not the child has or is at risk of having Celiac disease.

27. The method of claim 26, wherein the assessing comprises:
identifying the child as
(i) having or at risk of having Celiac disease if the T cell response to the composition is elevated compared to a control T cell response, or
(ii) not having or not at risk of having Celiac disease if the T cell response to the composition is reduced compared to the control T cell response or the same as the control T cell response.

28. The method of claim 26, wherein the composition comprises at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 5), and EQPIPEQPQ (SEQ ID NO: 6), or wherein the composition comprises at least one of:
(i) a first peptide comprising the amino acid sequence PFPQPELPY (SEQ ID NO: 1) and PQPELPYPQ (SEQ ID NO: 2),
(ii) a second peptide comprising the amino acid sequence PFPQPEQPF (SEQ ID NO: 3) and PQPEQPFPW (SEQ ID NO: 4), and
(iii) a third peptide comprising the amino acid sequence PIPEQPQPY (SEQ ID NO: 5), or a third peptide comprising the amino acid sequence PIPEQPQPY (SEQ ID NO: 5) and EQPIPEQPQ (SEQ ID NO: 6).

29-30. (canceled)

31. The method of claim 26, wherein the step of determining comprises contacting the sample with the composition and measuring a T cell response to the composition, optionally wherein measuring a T cell response to the composition comprises measuring a level of a cytokine in the sample.

32. (canceled)

33. The method of claim 31, wherein the cytokine is interferon-gamma, optionally wherein measuring comprises an enzyme-linked immunosorbent assay (ELISA) or an enzyme-linked immunosorbent spot (ELISpot) assay.

34. (canceled)

35. The method of claim 26, wherein
(i) the at least one peptide or the first, second, and/or third peptide are each independently 8-50 amino acids in length;
(ii) the first peptide comprises LQPFPQPQLPYPQPQ (SEQ ID NO: 7); the second peptide comprises QPFPQPQQPFPWQP (SEQ ID NO: 8); and the third peptide comprises PQQPIPQQPQPYPQQ (SEQ ID NO: 9), optionally wherein the first, second, and/or third peptide are each independently 15-30 amino acids in length
(iii) the first peptide comprises the amino acid sequence ELQPFPQPELPYPQPQ (SEQ ID NO: 10), wherein the N-terminal glutamate is a pyroglutamate and the C-terminal glutamine is amidated; the second peptide comprises the amino acid sequence EQPFPQPEQPFPWQP (SEQ ID NO: 11), wherein the N-terminal glutamate is a pyroglutamate and the C-terminal proline is amidated; and the third peptide comprises the amino acid sequence EPEQPIPEQPQPYPQQ (SEQ ID NO: 12), wherein the N-terminal glutamate is a pyroglutamate and the C-terminal glutamine is amidated;
(iv) the composition comprises the first and second peptide, the first and third peptide, or the second and third peptide;
(v) the composition comprises the first, second, and third peptide; or
(vi) the sample comprises whole blood or peripheral blood mononuclear cells.

36-43. (canceled)

44. The method of claim 26, wherein the method further comprises administering a composition comprising wheat, rye, or barley, or a peptide thereof, to the child prior to determining the T cell response.

45. The method of claim 44, wherein the composition comprising wheat, rye, or barley, or a peptide thereof, is administered to the child more than once prior to determining the T cell response, optionally wherein the composition comprising wheat, rye, or barley is administered to the child at least once a day for three days.

46. (canceled)

47. The method of claim 44, wherein the sample comprising the T cell is obtained from the child after the administration of the composition comprising wheat, rye, or barley, or a peptide thereof.

48. The method of claim 44, wherein the composition comprising wheat, rye, or barley is administered to the child via oral administration
optionally wherein (i) the composition comprising wheat, rye or barley, or a peptide thereof is a foodstuff, and/or (ii) the sample is obtained from the child 6 days after the oral administration.

49-50. (canceled)

51. The method of claim 26, wherein the method further comprises
(i) treating the child if identified as having or at risk of having Celiac disease or providing information to the child or the child's caregiver about a treatment; and/or
(ii) a step of recommending a gluten-free diet if the child is identified as having or at risk of having Celiac disease or providing information to the child or the child's caregiver about such a diet,
optionally wherein the child is HLA-DQ2.5 positive.

52-53. (canceled)


SEQUENCE LISTING (173 sequences)

SEQ ID NO: 1 - protein, 9 aa, Artificial Sequence
  Synthetic Polypeptide
PFPQPELPY

SEQ ID NO: 2 - protein, 9 aa, Artificial Sequence
  Synthetic Polypeptide
PQPELPYPQ

SEQ ID NO: 3 - protein, 9 aa, Artificial Sequence
  Synthetic Polypeptide
PFPQPEQPF

SEQ ID NO: 4 - protein, 9 aa, Artificial Sequence
  Synthetic Polypeptide
PQPEQPFPW

SEQ ID NO: 5 - protein, 9 aa, Artificial Sequence
  Synthetic Polypeptide
PIPEQPQPY

SEQ ID NO: 6 - protein, 9 aa, Artificial Sequence
  Synthetic Polypeptide
EQPIPEQPQ

SEQ ID NO: 7 - protein, 15 aa, Artificial Sequence
  Synthetic Polypeptide
LQPFPQPQLPYPQPQ

SEQ ID NO: 8 - protein, 14 aa, Artificial Sequence
  Synthetic Polypeptide
QPFPQPQQPFPWQP

SEQ ID NO: 9 - protein, 15 aa, Artificial Sequence
  Synthetic Polypeptide
PQQPIPQQPQPYPQQ

SEQ ID NO: 10 - protein, 16 aa, Artificial Sequence
  Synthetic Polypeptide
  misc_feature (1)..(1): Xaa is pyroglutamate
XLQPFPQPELPYPQPQ

SEQ ID NO: 11 - protein, 15 aa, Artificial Sequence
  Synthetic Polypeptide
  misc_feature (1)..(1): Xaa is pyroglutamate
XQPFPQPEQPFPWQP

SEQ ID NO: 12 - protein, 16 aa, Artificial Sequence
  Synthetic Polypeptide
  misc_feature (1)..(1): Xaa is pyroglutamate
XPEQPIPEQPQPYPQQ

SEQ ID NO: 13 - protein, 9 aa, Artificial Sequence
  Synthetic Polypeptide
PFPQPQLPY

SEQ ID NO: 14 - protein, 9 aa, Artificial Sequence
  Synthetic Polypeptide
PFPQPDLPY

SEQ ID NO: 15 - protein, 9 aa, Artificial Sequence
  Synthetic Polypeptide
PFPQPDLPY

SEQ ID NO: 16 - protein, 9 aa, Artificial Sequence
  Synthetic Polypeptide
PQPDLPYPQ

SEQ ID NO: 17 - protein, 9 aa, Artificial Sequence
  Synthetic Polypeptide
PFPQPDQPF

SEQ ID NO: 18 - protein, 9 aa, Artificial Sequence
  Synthetic Polypeptide
PQPDQPFPW

SEQ ID NO: 19 - protein, 9 aa, Artificial Sequence
  Synthetic Polypeptide
PIPDQPQPY

SEQ ID NO: 20 - protein, 15 aa, Artificial Sequence
  Synthetic Polypeptide
LQPFPQPDLPYPQPQ

SEQ ID NO: 21 - protein, 14 aa, Artificial Sequence
  Synthetic Polypeptide
QPFPQPDQPFPWQP

SEQ ID NO: 22 - protein, 15 aa, Artificial Sequence
  Synthetic Polypeptide
PQQPIPDQPQPYPQQ

SEQ ID NO: 23 - protein, 9 aa, Artificial Sequence
  Synthetic Polypeptide
FPQPELPYP

SEQ ID NO: 24 - protein, 9 aa, Artificial Sequence
  Synthetic Polypeptide
PQPQLPYPQ

SEQ ID NO: 25 - protein, 9 aa, Artificial Sequence
  Synthetic Polypeptide
PFPQPQQPF

SEQ ID NO: 26 - protein, 9 aa, Artificial Sequence
  Synthetic Polypeptide
PQPQQPFPW

SEQ ID NO: 27 - protein, 9 aa, Artificial Sequence
  Synthetic Polypeptide
PIPQQPQPY

SEQ ID NO: 28 - protein, 15 aa, Artificial Sequence
  Synthetic Polypeptide
LQPFPQPQLPYPQPQ

SEQ ID NO: 29 - protein, 14 aa, Artificial Sequence
  Synthetic Polypeptide
QPFPQPQQPFPWQP

SEQ ID NO: 30 - protein, 15 aa, Artificial Sequence
  Synthetic Polypeptide
PQQPIPQQPQPYPQQ

SEQ ID NO: 31 - protein, 13 aa, Artificial Sequence
  Synthetic Polypeptide
FPQPEQEFPQPQQ

SEQ ID NO: 32 - protein, 13 aa, Artificial Sequence
  Synthetic Polypeptide
EQPFPEQPFPEQP

SEQ ID NO: 33 - protein, 13 aa, Artificial Sequence
  Synthetic Polypeptide
ELPLQPEQPFPQP

SEQ ID NO: 34 - protein, 13 aa, Artificial Sequence
  Synthetic Polypeptide
FPQPQQQFPQPQQ

SEQ ID NO: 35 - protein, 14 aa, Artificial Sequence
  Synthetic Polypeptide
FPEQPIPEQPQPYP

SEQ ID NO: 36 - protein, 14 aa, Artificial Sequence
  Synthetic Polypeptide
FPQSEQPEQPFPQP

SEQ ID NO: 37 - protein, 14 aa, Artificial Sequence
  Synthetic Polypeptide
GLERPWQEQPLPPQ

SEQ ID NO: 38 - protein, 14 aa, Artificial Sequence
  Synthetic Polypeptide
FPEQPVPEQPQPYP

SEQ ID NO: 39 - protein, 14 aa, Artificial Sequence
  Synthetic Polypeptide
FPQTEQPEQPFPQP

SEQ ID NO: 40 - protein, 14 aa, Artificial Sequence
  Synthetic Polypeptide
GLERPWQQQPLPPQ

SEQ ID NO: 41 - protein, 14 aa, Artificial Sequence
  Synthetic Polypeptide
FSFSEQPEQPFPLQ

SEQ ID NO: 42 - protein, 13 aa, Artificial Sequence
  Synthetic Polypeptide
IIPEQPEQPFPLQ

SEQ ID NO: 43 - protein, 15 aa, Artificial Sequence
  Synthetic Polypeptide
GQPGYYPTSPQQIGQ

SEQ ID NO: 44 - protein, 16 aa, Artificial Sequence
  Synthetic Polypeptide
NPLQPEQPFPLQPQPP

SEQ ID NO: 45 - protein, 13 aa, Artificial Sequence
  Synthetic Polypeptide
IISEQPEQPFPLQ

SEQ ID NO: 46 - protein, 15 aa, Artificial Sequence
  Synthetic Polypeptide
GQQGYYPISPQQSGQ

SEQ ID NO: 47 - protein, 16 aa, Artificial Sequence
  Synthetic Polypeptide
PEQPFPEQPQPYPQQP

SEQ ID NO: 48 - protein, 13 aa, Artificial Sequence
  Synthetic Polypeptide
LPFPQPEQPFVVV

SEQ ID NO: 49 - protein, 13 aa, Artificial Sequence
  Synthetic Polypeptide
GQSGYYPTSPQQS

SEQ ID NO: 50 - protein, 14 aa, Artificial Sequence
  Synthetic Polypeptide
PEQPFQPEQPFPQQ

SEQ ID NO: 51 - protein, 13 aa, Artificial Sequence
  Synthetic Polypeptide
PAPIQPEQPFPQQ

SEQ ID NO: 52 - protein, 14 aa, Artificial Sequence
  Synthetic Polypeptide
IQVDPSGEVEWPQQ

SEQ ID NO: 53 - protein, 16 aa, Artificial Sequence
  Synthetic Polypeptide
PEQPQPFPEQPVPQQP

SEQ ID NO: 54 - protein, 13 aa, Artificial Sequence
  Synthetic Polypeptide
PEQIIPEQPEQPS

SEQ ID NO: 55 - protein, 14 aa, Artificial Sequence
  Synthetic Polypeptide
IQVDPSGEVQWPQQ

SEQ ID NO: 56 - protein, 13 aa, Artificial Sequence
  Synthetic Polypeptide
PFPLQPEQPFPWQ

SEQ ID NO: 57 - protein, 13 aa, Artificial Sequence
  Synthetic Polypeptide
PEQPFPEQPEQII

SEQ ID NO: 58 - protein, 14 aa, Artificial Sequence
  Synthetic Polypeptide
IQVDPSGQVEWPQQ

SEQ ID NO: 59 - protein, 13 aa, Artificial Sequence
  Synthetic Polypeptide
PFPWQPEQPFPQP

SEQ ID NO: 60 - protein, 13 aa, Artificial Sequence
  Synthetic Polypeptide
PEQPFPEQPQQII

SEQ ID NO: 61 - protein, 14 aa, Artificial Sequence
  Synthetic Polypeptide
IQVDPSGQVQWPQQ

SEQ ID NO: 62 - protein, 16 aa, Artificial Sequence
  Synthetic Polypeptide
PQPFPEQPIPEQPQPY

SEQ ID NO: 63 - protein, 13 aa, Artificial Sequence
  Synthetic Polypeptide
PEQPYPEQPFPQQ

SEQ ID NO: 64 - protein, 13 aa, Artificial Sequence
  Synthetic Polypeptide
LPYPQPELPYPQP

SEQ ID NO: 65 - protein, 16 aa, Artificial Sequence
  Synthetic Polypeptide
PQPYPEQPQPFPQQPP

SEQ ID NO: 66 - protein, 13 aa, Artificial Sequence
  Synthetic Polypeptide
PFLLQPEQPFSQP

SEQ ID NO: 67 - protein, 13 aa, Artificial Sequence
  Synthetic Polypeptide
LPYPQPQLPYPQP

SEQ ID NO: 68 - protein, 13 aa, Artificial Sequence
  Synthetic Polypeptide
QEFPQPEQPFPQQ

SEQ ID NO: 69 - protein, 13 aa, Artificial Sequence
  Synthetic Polypeptide
PFPEQPEQIIPQQ

SEQ ID NO: 70 - protein, 14 aa, Artificial Sequence
  Synthetic Polypeptide
LQPFPQPELPFPQP

SEQ ID NO: 71 - protein, 15 aa, Artificial Sequence
  Synthetic Polypeptide
QPFPEQPFPEQPQPY

SEQ ID NO: 72 - protein, 13 aa, Artificial Sequence
  Synthetic Polypeptide
PFPEQPEQIISQQ

SEQ ID NO: 73 - protein, 14 aa, Artificial Sequence
  Synthetic Polypeptide
LQPFPQPELPYLQP

SEQ ID NO: 74 - protein, 13 aa, Artificial Sequence
  Synthetic Polypeptide
QPFPQPEQPFPLQ

SEQ ID NO: 75 - protein, 13 aa, Artificial Sequence
  Synthetic Polypeptide
PFPEQPEQPFPQQ

SEQ ID NO: 76 - protein, 14 aa, Artificial Sequence
  Synthetic Polypeptide
LQPFPQPELPYPQP

SEQ ID NO: 77 - protein, 13 aa, Artificial Sequence
  Synthetic Polypeptide
QPFPQPEQPFRQQ

SEQ ID NO: 78 - protein, 13 aa, Artificial Sequence
  Synthetic Polypeptide
PFPERPEQPFPQP

SEQ ID NO: 79 - protein, 14 aa, Artificial Sequence
  Synthetic Polypeptide
LQPFPQPELPYSQP

SEQ ID NO: 80 - protein, 13 aa, Artificial Sequence
  Synthetic Polypeptide
QPFPQPEQPFSWQ

SEQ ID NO: 81 - protein, 13 aa, Artificial Sequence
  Synthetic Polypeptide
PFPLQPEQPFSQP

SEQ ID NO: 82 - protein, 14 aa, Artificial Sequence
  Synthetic Polypeptide
LQPFPQPQLPYLQP

SEQ ID NO: 83 - protein, 13 aa, Artificial Sequence
  Synthetic Polypeptide
QPFPQPEQPIPYQ

SEQ ID NO: 84 - protein, 15 aa, Artificial Sequence
  Synthetic Polypeptide
PFPLQPEQPVPEQPQ

SEQ ID NO: 85 - protein, 14 aa, Artificial Sequence
  Synthetic Polypeptide
LQPFPQPQLPYPQP

SEQ ID NO: 86 - protein, 13 aa, Artificial Sequence
  Synthetic Polypeptide
QPQPFPEQPIPLQ

SEQ ID NO: 87 - protein, 13 aa, Artificial Sequence
  Synthetic Polypeptide
PTPIQPEQPFPQR

SEQ ID NO: 88 - protein, 14 aa, Artificial Sequence
  Synthetic Polypeptide
LQPFPQPQLPYSQP

SEQ ID NO: 89 - protein, 13 aa, Artificial Sequence
  Synthetic Polypeptide
QPQPFPEQPIPQQ

SEQ ID NO: 90 - protein, 13 aa, Artificial Sequence
  Synthetic Polypeptide
QLFPLPEQPFPQP

SEQ ID NO: 91 - protein, 15 aa, Artificial Sequence
  Synthetic Polypeptide
LQQPFPQPQLPFPQP

SEQ ID NO: 92 - protein, 13 aa, Artificial Sequence
  Synthetic Polypeptide
QPQPYPEQPQPYP

SEQ ID NO: 93 - protein, 15 aa, Artificial Sequence
  Synthetic Polypeptide
QPEQPFPLQPEQPVP

SEQ ID NO: 94 - protein, 16 aa, Artificial Sequence
  Synthetic Polypeptide
LQQQCSPVAMPQRLAR

SEQ ID NO: 95 - protein, 13 aa, Artificial Sequence
  Synthetic Polypeptide
SYPVQPEQPFPQP

SEQ ID NO: 96 - protein, 13 aa, Artificial Sequence
  Synthetic Polypeptide
QPFPQPEQELPLQ

SEQ ID NO: 97 - protein, 13 aa, Artificial Sequence
  Synthetic Polypeptide
PEQPFPEQPEQPF

SEQ ID NO: 98 - protein, 13 aa, Artificial Sequence
  Synthetic Polypeptide
QPFPQPEQPFPQS

SEQ ID NO: 99 - protein, 13 aa, Artificial Sequence
  Synthetic Polypeptide
PEQTFPEQPQLPF

SEQ ID NO: 100 - protein, 13 aa, Artificial Sequence
  Synthetic Polypeptide
QPFPQPEQPIPQQ

SEQ ID NO: 101 - protein, 13 aa, Artificial Sequence
  Synthetic Polypeptide
PFPEQPEQEFPQP

SEQ ID NO: 102 - protein, 14 aa, Artificial Sequence
  Synthetic Polypeptide
YQPYPEQEQPILQQ

SEQ ID NO: 103 - protein, 13 aa, Artificial Sequence
  Synthetic Polypeptide
QPFPQPEQPTPIQ

SEQ ID NO: 104 - protein, 13 aa, Artificial Sequence
  Synthetic Polypeptide
PFPEQPEQPFPQP

SEQ ID NO: 105 - protein, 14 aa, Artificial Sequence
  Synthetic Polypeptide
YQPYPEQQQPILQQ

SEQ ID NO: 106 - protein, 13 aa, Artificial Sequence
  Synthetic Polypeptide
QPFPQPEQQLPLQ

SEQ ID NO: 107 - protein, 13 aa, Artificial Sequence
  Synthetic Polypeptide
PFPEQPEQPYPQQ

SEQ ID NO: 108 - protein, 13 aa, Artificial Sequence
  Synthetic Polypeptide
QSIPQPEQPFPQP

SEQ ID NO: 109 - protein, 13 aa, Artificial Sequence
  Synthetic Polypeptide
PFPLQPEQPFPQP

SEQ ID NO: 110 - protein, 12 aa, Artificial Sequence
  Synthetic Polypeptide
VGPSGEVEWPQQ

SEQ ID NO: 111 - protein, 13 aa, Artificial Sequence
  Synthetic Polypeptide
PFPLQPEQPFPQQ

SEQ ID NO: 112 - protein, 14 aa, Artificial Sequence
  Synthetic Polypeptide
YPEQPFPEQPEQPY

SEQ ID NO: 113 - protein, 13 aa, Artificial Sequence
  Synthetic Polypeptide
PFPLQPQQPFPQP

SEQ ID NO: 114 - protein, 15 aa, Artificial Sequence
  Synthetic Polypeptide
  misc_feature (1)..(1): Xaa is pyroglutamate
XQQPQQSFPEQERPF

SEQ ID NO: 115 - protein, 13 aa, Artificial Sequence
  Synthetic Polypeptide
YSPYQPEQPFPQP

SEQ ID NO: 116 - protein, 13 aa, Artificial Sequence
  Synthetic Polypeptide
PFPLQPQQPFPQQ

SEQ ID NO: 117 - protein, 15 aa, Artificial Sequence
  Synthetic Polypeptide
  misc_feature (1)..(1): Xaa is pyroglutamate
  misc_feature (2)..(2): Xaa is Leu or Ile
  misc_feature (8)..(8): Xaa is Leu or Ile
XXPQQQQXPEQPQQF

SEQ ID NO: 118 - protein, 13 aa, Artificial Sequence
  Synthetic Polypeptide
PFPQQPQQPFPQP

SEQ ID NO: 119 - protein, 15 aa, Artificial Sequence
  Synthetic Polypeptide
  misc_feature (1)..(1): Xaa is pyroglutamate
XQQSEESEQPFQPQP

SEQ ID NO: 120 - protein, 13 aa, Artificial Sequence
  Synthetic Polypeptide
PFPQQPQQPYPQQ

SEQ ID NO: 121 - protein, 15 aa, Artificial Sequence
  Synthetic Polypeptide
  misc_feature (1)..(1): Xaa is pyroglutamate
XQPPFSEEQEQPLPQ

SEQ ID NO: 122 - protein, 13 aa, Artificial Sequence
  Synthetic Polypeptide
PFPQQPQQQFPQP

SEQ ID NO: 123 - protein, 16 aa, Artificial Sequence
  Synthetic Polypeptide
  misc_feature (1)..(1): Xaa is pyroglutamate
XQPPFSEQQESPSFSQ

SEQ ID NO: 124 - protein, 13 aa, Artificial Sequence
  Synthetic Polypeptide
PFPWQPEQPFPQQ

SEQ ID NO: 125 - protein, 14 aa, Artificial Sequence
  Synthetic Polypeptide
  misc_feature (1)..(1): Xaa is pyroglutamate
XGIIPEQPAQLEGI

SEQ ID NO: 126 - protein, 13 aa, Artificial Sequence
  Synthetic Polypeptide
PFPWQPQQPFPQQ

SEQ ID NO: 127 - protein, 15 aa, Artificial Sequence
  Synthetic Polypeptide
  misc_feature (1)..(1): Xaa is pyroglutamate
XQPFRPEQPYPQPQP

SEQ ID NO: 128 - protein, 13 aa, Artificial Sequence
  Synthetic Polypeptide
PIPEQPEQPFPLQ

SEQ ID NO: 129 - protein, 16 aa, Artificial Sequence
  Synthetic Polypeptide
QPQQPQQSFPQQQRPF

SEQ ID NO: 130 - protein, 13 aa, Artificial Sequence
  Synthetic Polypeptide
PIPQQPQQPFPLQ

SEQ ID NO: 131 - protein, 21 aa, Artificial Sequence
  Synthetic Polypeptide
  misc_feature (3)..(3): Xaa is Leu or Ile
  misc_feature (8)..(8): Xaa is Leu or Ile
  misc_feature (14)..(14): Xaa is Leu or Ile
QQXSQPQXPQQQQXPQQPQQF

SEQ ID NO: 132 - protein, 13 aa, Artificial Sequence
  Synthetic Polypeptide
PQPFLPELPYPQP

SEQ ID NO: 133 - protein, 21 aa, Artificial Sequence
  Synthetic Polypeptide
QPQPFPQQSEQSQQPFQPQPF

SEQ ID NO: 134 - protein, 13 aa, Artificial Sequence
  Synthetic Polypeptide
PQPFLPQLPYPQP

SEQ ID NO: 135 - protein, 15 aa, Artificial Sequence
  Synthetic Polypeptide
QQPPFSQQQQQPLPQ

SEQ ID NO: 136 - protein, 13 aa, Artificial Sequence
  Synthetic Polypeptide
PQQPFPQQPQQPF

SEQ ID NO: 137 - protein, 19 aa, Artificial Sequence
  Synthetic Polypeptide
QQQQPPFQQQQSPFSQQQQ

SEQ ID NO: 138 - protein, 13 aa, Artificial Sequence
  Synthetic Polypeptide
PQQTFPQQPQLPF

SEQ ID NO: 139 - protein, 15 aa, Artificial Sequence
  Synthetic Polypeptide
VQGQGIIQPQQPAQL

SEQ ID NO: 140 - protein, 13 aa, Artificial Sequence
  Synthetic Polypeptide
PTPIQPEQPFPQQ

SEQ ID NO: 141 - protein, 14 aa, Artificial Sequence
  Synthetic Polypeptide
PFRPQQPYPQPQPQ

SEQ ID NO: 142 - protein, 13 aa, Artificial Sequence
  Synthetic Polypeptide
PTPIQPQQPFPQQ

SEQ ID NO: 143 - protein, 13 aa, Artificial Sequence
  Synthetic Polypeptide
QAFPQPEQTFPHQ

SEQ ID NO: 144 - protein, 13 aa, Artificial Sequence
  Synthetic Polypeptide
QAFPQPQQTFPHQ

SEQ ID NO: 145 - protein, 12 aa, Artificial Sequence
  Synthetic Polypeptide
QFIQPEQPFPQQ

SEQ ID NO: 146 - protein, 12 aa, Artificial Sequence
  Synthetic Polypeptide
QFIQPQQPFPQQ

SEQ ID NO: 147 - protein, 13 aa, Artificial Sequence
  Synthetic Polypeptide
QPFPQLEQPEQPF

SEQ ID NO: 148 - protein, 13 aa, Artificial Sequence
  Synthetic Polypeptide
QPFPQLQQPQQPF

SEQ ID NO: 149 - protein, 13 aa, Artificial Sequence
  Synthetic Polypeptide
QPFPQPEQPFCQQ

SEQ ID NO: 150 - protein, 13 aa, Artificial Sequence
  Synthetic Polypeptide
QPFPQPEQPFPWQ

SEQ ID NO: 151 - protein, 13 aa, Artificial Sequence
  Synthetic Polypeptide
QPFPQPEQPFSQQ

SEQ ID NO: 152 - protein, 13 aa, Artificial Sequence
  Synthetic Polypeptide
QPFPQPEQPIPVQ

SEQ ID NO: 153 - protein, 13 aa, Artificial Sequence
  Synthetic Polypeptide
QPFPQPEQPQLPF

SEQ ID NO: 154 - protein, 13 aa, Artificial Sequence
  Synthetic Polypeptide
QPFPQPEQTFPQQ

SEQ ID NO: 155 - protein, 13 aa, Artificial Sequence
  Synthetic Polypeptide
QPFPQPQQPFCQQ

SEQ ID NO: 156 - protein, 13 aa, Artificial Sequence
  Synthetic Polypeptide
QPFPQPQQPFPWQ

SEQ ID NO: 157 - protein, 13 aa, Artificial Sequence
  Synthetic Polypeptide
QPFPQPQQPFSQQ

SEQ ID NO: 158 - protein, 13 aa, Artificial Sequence
  Synthetic Polypeptide
QPFPQPQQPIPVQ

SEQ ID NO: 159 - protein, 13 aa, Artificial Sequence
  Synthetic Polypeptide
QPFPQPQQPQLPF

SEQ ID NO: 160 - protein, 13 aa, Artificial Sequence
  Synthetic Polypeptide
QPFPQPQQTFPQQ

SEQ ID NO: 161 - protein, 13 aa, Artificial Sequence
  Synthetic Polypeptide
QPFTQPEQPTPIQ

SEQ ID NO: 162 - protein, 13 aa, Artificial Sequence
  Synthetic Polypeptide
QPFTQPQQPTPIQ

SEQ ID NO: 163 - protein, 13 aa, Artificial Sequence
  Synthetic Polypeptide
QQFSQPEQQFPQP

SEQ ID NO: 164 - protein, 13 aa, Artificial Sequence
  Synthetic Polypeptide
QQFSQPQQQFPQP

SEQ ID NO: 165 - protein, 13 aa, Artificial Sequence
  Synthetic Polypeptide
TIPEQPEQPFPLQ

SEQ ID NO: 166 - protein, 13 aa, Artificial Sequence
  Synthetic Polypeptide
TIPQQPQQPFPLQ

SEQ ID NO: 167 - protein, 16 aa, Artificial Sequence
  Synthetic Polypeptide
VAHAIIMHQQQQQQQE

SEQ ID NO: 168 - protein, 14 aa, Artificial Sequence
  Synthetic Polypeptide
YEVIRSLVLRTLPN

SEQ ID NO: 169 - protein, 11 aa, Artificial Sequence
  Synthetic Polypeptide
GIIQPQQPAQL

SEQ ID NO: 170 - protein, 26 aa, Artificial Sequence
  Synthetic Polypeptide
FLQPEQPFPEQPEQPYPEQPEQPFPQ

SEQ ID NO: 171 - protein, 15 aa, Artificial Sequence
  Synthetic Polypeptide
PQQPQQSFPQQQQPA

SEQ ID NO: 172 - protein, 14 aa, Artificial Sequence
  Synthetic Polypeptide
QPPFSEEQEQPLPQ

SEQ ID NO: 173 - protein, 9 aa, Artificial Sequence
  Synthetic Polypeptide
QGSFQPSQQ